SEQUENCE LISTING (85 sequences)

SEQ ID NO: 1 - DNA, 549 bp, Streptomyces hygroscopicus
  source 1..549: mol_type = genomic DNA
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatc

SEQ ID NO: 2 - protein, 183 aa, Streptomyces hygroscopicus
MSPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEI

SEQ ID NO: 3 - DNA, 2746 bp, Triticum aestivum
  source 1..2746: mol_type = genomic DNA
ggatttggagtagatctgtgggtgggtcgaggcgttattgctgttgatcgattgcagctcgttcggttagccgtggttgattaggcctgattgtagttaataccatctctcgtgattatgccatgattttcgagtagatctgccgattatgacgggattgctgtggggtcggttcgtcttagttctgcttggttgttattgtggatttttgattatgtaccgctcatgagcctgatatgttgaaggatacaccttcgtggtgttattgggatgcgatgcgattgctgttggttcgcttcatgtatctgttatagttagctactacagtagtttctgattttgtagatcctatgatccgtggaaggatacgcctgtggtttgctagcttaatagttcagcttttgtagttgcttgtagcgtccctagttgagcatcgaatttaactcttaaattcatgtcggcatgtcggtttgccgacattactgttcgtttctaggcttcttgacaagcttctcagtacaactagattacataagctcccactgaagctaaagattgctctggtagatatgctaatatattattctgctaaaatttagcactggatggtctgtcaatccattggcttgttctaggggccagctcctgggcagtttgctgtcagctctctagctgatttcatctctgttgcaatttcatctttgtagcatggtggccatctggttcatttattgtttgacgtactgcagatatccctgtacgctaacttactctggtttctgtctgctttgtatttacagatgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggttctcaggctccgtggtggtatgcagatatttgtcaagaccctcactggcaaaaccatcaccctcgaggttgagtcctcagacaccattgataatgtgaaggccaagatccaggacaaggagggtatcccaccggaccagcagcgtctcatctttgctggcaagcagctcgaggatggccgcacccttgcagactacaacatccagaaggagtccactctccacttggtgctcaggctccgtggtggtatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggtggccagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccggccggaacaatgttaacatgatatggttttgtttttgtttcaattttcaattattggtttgta

SEQ ID NO: 4 - DNA, 2989 bp, Triticum aestivum
  source 1..2989: mol_type = genomic DNA
ggatttggagtagatctgtgggtcgaggcgttattgccgttgatcgattgcatctcgttcggttagtcgtggctgattaggcctgattgtaggcaataccatctctcttgattatgccatgattttcgaggagatctgccgattataatgggattgctatggggtcggttcgtcttatctagttggtagttgttggttctgcttggttgttattgtggatttttgattatgtaccgctcatgagcctgattttttttaattatagttggttcggttcatctatctagttatagacttatagttggctagtttctgactttgtagcccaactttctgtggaacgatacacctgtgatttgctagcttaataattcagcttttgtagttgcttctagcgtccatagttttgcatcgaattcaactcttaaattcatgttggtttgccgatattactgttggcttctaggcttcttgacaagcttctcagtacaactagattacataagctcccactgaagctaaagattgcactggtagatatgctaatatataattctgctaaaatttagcactggatggtctgtcaatccgttggcttgttctagggccagctcctgtgcagtttgctgtcagctctctagctgatatcatcactgttgaaatttcaactttgtagcatgctggccatctggttcatctattgtttgacgtactgcagatatccctgtacagcaacttactctggttctgtctgctttgtattatttacagatgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggtgctcaggctccgtggtggcatgcagatcttcgtcaagaccctcacgggcaagaccatcacactggaggttgagtcctcggacaccatcgataacgtgaaggccaagatccaggacaaggagggcatccctccggaccagcagcgcctgatctttgctggcaagcagcttgaggatggccgtaccctggctgactacaacatccagaaggagtccacccttcacctggtgctgaggctccgtggtggcatgcagatatttgtgaagaccctcaccggcaagaccatcaccctggaggtggagtcatctgacaccatcgacaacgtcaaggcgaagatccaggacaaggagggcattcccccggaccagcagcgtcttatctttgctggcaaacagctcgaggatggccgcacccttgcggactacaacatccagaaagagtccaccctccacttggtgctcaggctccgtggtggtatgcagatttttgtcaagaccctgaccggcaagaccatcaccctggaggtcgagtcgtctgacaccattgacaatgtgaaggccaagatccaggacaaggagggcattcccccggaccagcagcgtctcatcttcgctggcaagcagcttgaggatggccgcacccttgcggattacaacatccagaaggagtccaccctccaccttgtcctccgccttcgtggaggcatgcaaatctttgtcaagaccctcactggcaagactatcaccttggaggtggagtcatctgacaccatcgacaacgtgaaggccaagatccaggacaaggagggcatccccccggaccagcagcgcctcatctttgctggtaagcagcttgaggatggccgcaccttggcagactacaacattcagaaggagtcaacccttcacttggtgctccgtctccgtggtggccagtaagctctggccatggagctgcttctgttatctgtcttcggtgtcctccaatggagtctggtctgtgtctgtcgttgcatgaactgtctttgtttctatatatatataccatactgtgatgcagtgttattgtttgtaagtgcaaacttctgctggtgtgcagcagctttgatgaacttcagaataagtgagccctgaattcggtccataattctgtttgtattgcctgatgtttgctgttgtgttataccgtggtttcttgctatcttactgatgcatttgacggcacatgttttctggttgatgtaatactttacttctatgctctgtcctttacggagaataggatcgcaactcagtacagctcgtgacccggctgatcttttccgatgatcagttggctgacgcgtagcacgtcccaaaaaataatgttcacatgatggtttcgttgtcccaagtaccatttcggactgtaatttttcacaagtgcaaaacttctaagttacatcatatttatgaaaaaaatcagggccatgcaaccatgtaaaagtcacattgcgcaacccatatgtcaatgttaactatgtatcaacatgatctgaatttgttcaaaaaaagaaaaaacatgatctgaatatgaatatatctattctaccacaagctgtttttgagttgctacctctctcgtgatacatcatcgctacttctctgctgataccttccattcctatccagtggttggttgacgagatagtgagtgaaaaggagaaaaaggaagatgaattaatcaaccaacgcttgttaacatctgaaaaagatgttgggtacaatgggaaggtaagggaggctgtataaatatgtggtagttcaacttcaaaaatatttgtgataaaggtgagacatttgatggcatggagaatttgagaccaaagttcacgtgcaatgttaggtgcaaagagttaattccgtcgaagactaaaattaatagtagttaaaattacacatgtgcatattatgttgtaaggagacaaggacccaatattatccgaacttcgatcttgttctgcgtaccctc

SEQ ID NO: 5 - DNA, 3248 bp, Triticum aestivum
  source 1..3248: mol_type = genomic DNA
ctccgatggccgctcacgcaccacaccaacggcgtgagccgacccgtccaactccaacggccaccaaccagccgaaccctcgacggcgtcacctttggaatctcctccccatatcctggattttttgcgtggtggggacggactccgcgaccgcctctcccctcccttttttttttgagggaagcctctccccttcctatccgctccggcccccacaagtcacccgtgccggtgccgtagcgcttcttccttctctccctcccgtccgagccggctcggctataaaatccccgccccctcccgtctcctctccacacatccgatccgatccaatccaatccagcccccctacgatctcaccgggaaacctccgatccgagcgaagcgaaggctctctcgtcctcctcctcaaggtatgcgaggctacggtttcttcttcgctctctcgtgcgtgcgtgcgtgcccttggatcgatcgttgctagcaccgccctcgatcctgctagtcgtatggatttggagtagatctgtgggtgggtcgaggcgttattgctgttgatcgattgcagctcgttcggttagtcgtggctgattaggcctgattgtagttttttttttgttttttaggcctgattgtagttaacacgtctctcttgattatgccgtgattttcgagcagatctgccgattatgatgggattgctgtggggtcggttcgtcttagttctgcttggttgttattgtggatttttgattatgtaccgctcatgagcctcatatgttgaagggtacaccttcgtggtattactgggatgcgatgcgattgctgttggttcggttcatctatctcgttatagacttagttagctagtttctgattttgtagccctatgatctgtggaaggatacacctatgatttgctagattaatagttcagctttgtagttgcttgtagcgtccgtagttgggcattgaattcaactcttaaattcatgtcggtttgccgatattactgttggtttctaggcttgttgacaagcttctcagtacaaccagattacatcagctcctattgaaactgcgctggtagatatgctattccctccgtcccataatataacagcgtttttgatactagtatagtgtcaaaaacactcttatattatgtcaatccattggcttgttctagtggccagctcctgggcagtttgctgtcagctctctagctgatttcatctctgttgcaatttcatctttgtagcatggtggccatctggttcatctattgtttgacgtactgcagatatccctgtactgtaacttaatctgcttctgtctgctttgtatttacagatgcagatctttgtgaagaccctcaccggcaagaccatcacccttgaggtcgagtcctctgacaccattgacaatgtcaaggcaaagatccaggacaaggagggcatccccccggaccagcagcgtctcatctttgctggcaagcagctcgaggatggccgcacccttgcggactacaacatccagaaggagtccactctccacttggtgctcaggctacgtggtggtatgcagatctttgtcaagaccctcaccggcaagaccatcaccctggaggttgagtcctcagacaccattgacaatgtgaaggccaagatccaggacaaggagggcatccccccggaccagcagcgcctgatctttgccggcaagcagctcgaggatggccgcacccttgcggattacaacatccagaaggagtccaccctccacttggtgctcaggctccgtggtggcatgcagatatttgtgaagaccctcaccggcaagaccatcaccctggaggtggagtcatctgacaccatcgacaacgtgaaggcaaagatccaggacaaggagggcattcccccggaccagcagcgcctcatctttgctggtaagcagcttgaggatggccgcaccttggcggactacaacattcagaaggagtcaacccttcacttggtgctccgtctccgtggtggccagtaagctcctggccatggagctgcttttgtctctgggttcacaagtctcgtcttcggtgtcctccaaaggagtctggtatatgtctgtcgttgcctgaactgtcttcgtttctataccgtactatgatgcagtgttatcgtttgtatctgcaaacttctgctggtgtgcagcagctttgatgaacttaatattaagtgagccctgaattcaatccataatattgtttgtcttgcctgatgtttcctgttgtgttatgtggtttcttgtgatatttctgatgcatttgatctgcttttggcacatgttttctggttggtgacctctatgctctatcctctgaaatgcgtgtgttcaaacagattttgttttttgtactcaactttctggcctgtagaatccctttgatcctcaaattttctaagacacgtggagtcccgtgtgctggtcgttgccccattttattcaaacatatgtgtgttcttcttgctgatcttgtgcttgtgttggctgcatgacccctgatgccctctcgcacaggcgcggctgctgtagacgcggcgacctccagccaaagacgtggggcaccggcgctgcaatgttggggcaccggcgctgcaatgttggggcgtccggtgacagtgagggtcgttccctgttgctggagttgccgttgcaaagtgggtggacgtgggccgctacaagggtctagcttgccacaatgacaataagacacgagctgaagttgcaacatgaggtcttgttataattgcaacaagtgaaagaggataggatcgtgagatcggactggtcacccggccaatcttttccaaagatcagccggccgatcttttccaaagatcagccagccgacgagtagcaagccctaagaaaacaatgctcacatggttttgtttgtcccaattttcaattattggtatgtagcattttggataatgtattttttcacaagtgcaaaacttccaagttacatcatttttatgaaaaaatctgggctatgcaatcgtataaaaatcgcgttgcgcaacccatatgccactttaactacgtaccaacatgatctaaaccttagtgtatccctgatctaccacaagctgtttccgagttgctatctctctcttgatacatcatcgctacctttctcttgatacctcccattcctatccagtggttggttgatgagatagtga

SEQ ID NO: 6 - DNA, 20 bp, Triticum aestivum
  source 1..20: mol_type = genomic DNA
ctccatggccaggagcttac

SEQ ID NO: 7 - DNA, 20 bp, Triticum aestivum
  source 1..20: mol_type = genomic DNA
cgtggtggccagtaagctcc

SEQ ID NO: 8 - DNA, 20 bp, Triticum aestivum
  source 1..20: mol_type = genomic DNA
caggagcttactggccacca

SEQ ID NO: 9 - DNA, 362 bp, Triticum aestivum
  source 1..362: mol_type = genomic DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 10 - DNA, 889 bp, synthetic construct
  misc_feature 1..889: note = TaU6_G1,G2,G3 mutiplex gRNA
  source 1..889: mol_type = other DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgaacaaagcaccagtggtctagtggtagaatagtaccctgccacggtacagacccgggttcgattcccggctggtgcactccatggccaggagcttacgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacgtggtggccagtaagctccgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacaggagcttactggccaccagttttagagctagaaatagcaagttaaaataaggctagtccgttatcaacttgaaaaagtggcaccgagtcggtgcttttttt

SEQ ID NO: 11 - DNA, 1992 bp, Zea mays
  source 1..1992: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 12 - DNA, 250 bp, Agrobacterium tumefaciens
  source 1..250: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag

SEQ ID NO: 13 - DNA, 4206 bp, synthetic construct
  misc_feature 1..4206: note = SV40 NLS-SpCas9-Nucleoplasmin NLS, maize optimised
  source 1..4206: mol_type = other DNA
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatcatatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaagcgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacaagaggccggcggcaaccaagaaggcgggccaggccaagaagaagaagtga

SEQ ID NO: 14 - DNA, 2043 bp, synthetic construct
  misc_feature 1..2043: note = Ubi7DL-BAR -Ub7DL donor repair fragment flanked by G3 gRNA target sites
  source 1..2043: mol_type = other DNA
ccgtctccgtggtggccagtaagctcctggccatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgacagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccgtctccgtggtggccagtaagctcctggccatg

SEQ ID NO: 15 - DNA, 2373 bp, Triticum aestivum
  source 1..2373: mol_type = genomic DNA
atgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 16 - protein, 790 aa, Triticum aestivum
MPRFSSTTPMSPPRLRLRLRLCARHSSSTSHPSRIWDPHAAFAAAAQRASSGTLTTEDAHHLFDELLRRGNPVQERPLNKFLAALARAPASASCCDGPALAVTLFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAIRLHGYATEGCLVDMINLFNSMATHCILPNCHIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 17 - DNA, 2367 bp, Triticum aestivum
  source 1..2367: mol_type = genomic DNA
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 18 - protein, 788 aa, Triticum aestivum
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 19 - DNA, 2379 bp, Triticum timopheevi
  source 1..2379: mol_type = genomic DNA
atgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatag

SEQ ID NO: 20 - protein, 792 aa, Triticum timopheevi
MPRFSSTTPMSPPRLLLRLGARHSSSTSHPSRIWDPHAAFAAATQRARSGTLTTEDAHHLFDELLRQGNPVQERPLTNFLAALARAPASAFCSDGPALAVALFGRLSRGAGRRVAQPNVFTYGVLMDCCCRARRLDLAIAFFARLLKTGLEANQVIFCTLLKGLCHAKRSDEALDVVLHRMPELGCTPNVVAYTTVIHGFLKEGQVGKACNLFHGMAQQGVAPDVVTYNSVIDALCKARAMDKAEYFLREMVDNGVVPNNVTYNSLIHGYSSLGHQKEAVRVLKEMTRQGIIPDVITCTSLMTFLCKNGKSKEAAEIFDSMATKGLKHDAVSYAILLHGYATEGCLVDMINLFNSMDRDCILPNCHIFNILIYAYAKSGKLDKAMLIFRDMQKQGVSPDAFTYSTLIHAFCKKGRLDDAMIKFNQMVDTGVRQGTAVYGSLIQGFCTHGDLVKGKELVTEMMNKGIPPPDIMFFHSIMQNLCTEGRVVEARDILGLIAHIGMRPNVCTFNILIGGYCLVRKMEDASKIFHDMMSYGLEPSNVTYGILINGYCKNRRIDDGLILFKEMLRKGLKPTTFNYNIILDGLFLAGRTVAAKEKFDEMVESGVSMCISTYSIVLRGLCRNNCSGEAITLFQTLSAMDVKFNIRIVNIMIDAFFRVQRKQEAKDLFAAITANGLVANVFTYSLMMTNLIKEGSVEEADTLFLSMEMSGCTSNSWMLNLIIRGLLEKGEIVKAGCYMSKVDAKSYSLEAKTVSLLIYLFSGKGKYREHIRLLPTKYQFLEEAATVEWFAI

SEQ ID NO: 21 - DNA, 3051 bp, synthetic construct
  misc_feature 1..3051: note = RH-Ubi7DL-RFL29a CDS
  source 1..3051: mol_type = other DNA
atgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagtggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 22 - DNA, 3093 bp, synthetic construct
  misc_feature 1..3093: note = RFL29a_RH-Ubi7DL CDS
  source 1..3093: mol_type = other DNA
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagtggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaaaaggagtccaccctccacctggttctcaggctccgtggtggtatgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggttctcaggctccgtggtggtatgcagatatttgtcaagaccctcactggcaaaaccatcaccctcgaggttgagtcctcagacaccattgataatgtgaaggccaagatccaggacaaggagggtatcccaccggaccagcagcgtctcatctttgctggcaagcagctcgaggatggccgcacccttgcagactacaacatccagaaggagtccactctccacttggtgctcaggctccgtggtggtatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggctaa

SEQ ID NO: 23 - DNA, 3063 bp, synthetic construct
  misc_feature 1..3063: note = RH-Ubi7DL-RFL79 CDS
  source 1..3063: mol_type = other DNA
atgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatag

SEQ ID NO: 24 - DNA, 3105 bp, synthetic construct
  misc_feature 1..3105: note = RFL79_RH-Ubi7DL CDS
  source 1..3105: mol_type = other DNA
atgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctataaaggagtccaccctccacctggttctcaggctccgtggtggtatgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggttctcaggctccgtggtggtatgcagatatttgtcaagaccctcactggcaaaaccatcaccctcgaggttgagtcctcagacaccattgataatgtgaaggccaagatccaggacaaggagggtatcccaccggaccagcagcgtctcatctttgctggcaagcagctcgaggatggccgcacccttgcagactacaacatccagaaggagtccactctccacttggtgctcaggctccgtggtggtatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggctaa

SEQ ID NO: 25 - DNA, 476 bp, synthetic construct
  misc_feature 1..476: note = pTaU6_G3 gRNA
  source 1..476: mol_type = other DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgcaggagcttactggccaccagtttcagagctatgctggaaacagcatagcaagttgaaataaggctagtccgttatcaacttgaaaaagtggcaccgagtcggtgcttttttt

SEQ ID NO: 26 - DNA, 3858 bp, synthetic construct
  misc_feature 1..3858: note = Ubi7DL-RFL29a -Ub7DL donor repair fragment flanked by G3 gRNA target sites
  source 1..3858: mol_type = other DNA
ccgtctccgtggtggccagtaagctcctggccatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagtggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagcagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccgtctccgtggtggccagtaagctcctggccatg

SEQ ID NO: 27 - DNA, 3870 bp, synthetic construct
  misc_feature 1..3870: note = Ubi7DL-RFL79 -Ub7DL donor repair fragment flanked by G3 gRNA target sites
  source 1..3870: mol_type = other DNA
ccgtctccgtggtggccagtaagctcctggccatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatagcagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccgtctccgtggtggccagtaagctcctggccatg

SEQ ID NO: 28 - DNA, 6504 bp, synthetic construct
  misc_feature 1..6504: note = Ubi7DL-RFL29a-Ubi-RFL79 -Ub7DL donor repair fragment flanked by G3 gRNA target sites
  source 1..6504: mol_type = other DNA
ccgtctccgtggtggccagtaagctcctggccatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagtggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaaaaggagtccaccctccacctggttctcaggctccgtggtggtatgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggttctcaggctccgtggtggtatgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatagcagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccgtctccgtggtggccagtaagctcctggccatg

SEQ ID NO: 29 - DNA, 6936 bp, Triticum aestivum
  source 1..6936: mol_type = genomic DNA
atgggatccacacatttgcccatcgtcggctttaatgcctcaacaacaccatcgctatccactattcgcccggtaaattcagctggtgctgcattccaaccatctgccccttctagaacctccaagaagaaaagtcgtcgtgttcagtcattaagggatggaggcgatggaggcgtgtcagaccctaaccagtctattcgccatggtcttgccggcatcattgacctcccaaaggagggcacatcagctccggaagtggatatttcacatgggtccgaagaacccaggggctcctaccaaatgaatgggatactgaatgaagcacataatgggaggcatgcttcgctgtctaaggttgttgaattttgtacggcattgggcggcaaaacaccaattcacagtgtattagttgcgaacaatggaatggcagcagctaagttcatgcggagtgtccgaacatgggctaatgaaacatttgggtcagagaaggcaattcagttgatagctatggctactccagaagacatgaggataaatgcagagcacattagaattgctgatcaatttgttgaagtacccggtggaacaaacaataacaactatgcaaatgtccaactcatagtggagatagcagtgagaaccggtgtttctgctgtttggcctggttggggccatgcatctgagaatcctgaacttccagatgcactaaatgcaaacggaattgtttttcttgggccaccatcatcatcaatgaacgcactaggtgacaaggttggttcagctctcattgctcaagcagcaggggttccgactcttccttggagtggatcacaggtggaaattccattagaagtttgtttggactcgatacctgcggagatgtataggaaagcttgtgttagtactacagaggaagcacttgcgagttgtcagatgattgggtatcccgccatgattaaagcatcatggggtggtggtggtaaagggatccgaaaggttaataatgacgatgatgtcagagcactgtttaagcaagtgcaaggtgaagttcctggctccccaatatttatcatgagacttgcatctcagagtcgacatcttgaagttcagttgctttgtgatcaatatggcaatgtagctgcgcttcacagtcgtgactgcagtgtgcaacggcgacaccaaaagattattgaggaaggaccagttactgttgctcctcgcgagacagtgaaagagctagagcaagcagcaaggaggcttgctaaggctgtgggttatgttggtgctgctactgttgaatatctctacagcatggagactggtgaatactattttctggaacttaatccacggttgcaggttgagcatccagtcaccgagtggatagctgaagtaaacttgcctgcagctcaagttgcagttggaatgggtatacccctttggcaggttccagagatcagacgtttctatggaatggacaatggaggaggctatgacatttggaggacaacagcagctcttgctactccatttaacttcgatgaagtggattctcaatggccaaagggtcattgtgtagcagttaggataaccagtgaggatccagatgacggattcaagcctaccggtggaaaagtaaaggagatcagttttaaaagcaagccaaatgtttgggcctatttctctgttaagtccggtggaggcattcatgaatttgctgattctcagtttggacatgtttttgcatatggagtgtctagagcagcagcaataaccaacatgtctcttgcgctaaaagagattcaaattcgtggagaaattcattcaaatgttgattacacagttgatctcttgaatgcctcagacttcaaagaaaacaggattcatactggctggctggataacagaatagcaatgcgagtccaagctgagagacctccctggtatatttcagtggttggaggagctctatataaaacaataacgagcaacacagacactgtttctgaatatgttagctatctcgtcaagggtcagattccaccaaagcatatatcccttgtccattcaactgtttctttgaatatagaggaaagcaaatatacaattgaaactataaggagcggacagggtagctacagattgcgaatgaatggatcagttattgaagcaaatgtccaaacattatgtgatggtggacttttaatgcagttggatggaaacagccatgtaatttatgctgaagaagaggccggtggtacacggcttctaattgatggaaagacatgcttgttacagaatgatcacgatccttcaaggttattagctgagacaccctgcaaacttcttcgtttcttggttgccgatggtgctcatgttgaagctgatgtaccatatgcggaagttgaggttatgaagatgtgcatgcccctcttgtcacctgctgctggtgtcattaatgttttgttgtctgagggccagcctatgcaggctggtgatcttatagcaagacttgatcttgatgacccttctgctgtgaagagagctgagccatttaacgggtctttcccagaaatgagccttcctattgctgcttctggccaagttcacaaaagatgtgccacaagcttgaatgctgctcggatggtccttgcaggatatgatcacccgatcaacaaagttgtacaagatctggtatcctgtctagatgctcctgagcttcctttcctacaatgggaagagcttatgtctgttttagcaactagacttccaaggcttcttaagagcgagttggagggtaaatacagtgaatataagttaaatgttggccatgggaagagcaaggatttcccttccaagatgctaagagagataatcgaggaaaatcttgcacatggttctgagaaggaaattgctacaaatgagaggcttgttgagcctcttatgagcctactgaagtcatatgagggtggcagagaaagccatgcacactttattgtgaagtcccttttcgaggactatctctcggttgaggaactattcagtgatggcattcagtctgatgtgattgagcgcctgcgccaacaacatagtaaagatctccagaaggttgtagacattgtgttgtctcaccagggtgtgagaaacaaaactaagctgatactaacactcatggagaaactggtctatccaaaccctgctgcctacaaggatcagttgactcgcttttcttccctcaatcacaaaagatattataagttggcccttaaagctagcgagcttcttgaacaaaccaagcttagtgagctccgcacaagcattgcaaggagcctttcagaacttgagatgtttactgaagaaaggacggccattagtgagatcatgggagatttagtgactgccccgctgccagttgaagatgcactggtttctttgtttgattgtagtgatcacactcttcagcagagggtgatcgagacgtacatatctcgattataccagcctcatcttgtcaaggatagtatccagctgaaatatcaggaatctggtgttattgctttatgggaattcgctgaagcgcattcagagaagagattgggtgctatggttattgtgaagtcgttagaatctgtatcagcagcaattggagctgcactaaaggatacatcacgctatgcaagctctgagggtaacataatgcatattgctttattgggtgctgataatcaaatgcatggaactgaagacagtggtgataacgatcaagctcaagtcaggatagacaaactttctgcaacactggaacaaaatactgtcacagctgatctctgtgatgctggtgtgaaggttattagttgcattgttcaaagggatggagcactcatgcctatgcgccataccttcctcttgtcggatgaaaagctttgttatgaggaagagccggttctccggcatgtggagcctcctctttctgctcttcttgagttgggtaagttgaaagtgaaaggatacaatgaggtgaagtatacaccgtcacgtgatcgtcagtggaacatatacacacttagaaatacagaaaaccccaaaatgttgcacagggtgtttttccgaactcttgtcaggcaacccggtgcttccaacaaattcacatcaggccacatcagtgatgttgaggtgggaggagctgaggaatctctttcatttacatcgagcagcatattaagatcgctgatgactgctatagaagagttggagcttcacgcgattaggacaggtcactctcatatgtttttgtgcatattgaaagagcaaaagcttcttgatcttgttcctgtttcagggaacaaagttgtggatattggccaagatgaagctactgcatgctcgcttctgaaagaaatggctctacagatacatgaacttgtgggtgcaaggatgcatcatctttctgtatgccaatgggaggtgaaacttaagttggacagcgatgggcctgcaagtggtacctggagagtcgtaacaaccaatgttactagtcacacctgcactgtcgatatctaccgtgaggttgaagatacagaatcacagaaactagtataccactctgctccatcgtcatctggtcctttgcatggcgttgcactgaatactccatatcagcctttgagtgttattgatctgaaacgttgctccgctagaaacaacagaactacatactgctatgattttccgttggcatttgaaactgcagtgcagaagtcatggtctaacatttctagtgacaataaccgatgttatgttaaagcaacggagctggtgtttgctcaaaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcgtgcttccgtgttggatggtctgatgatggcagccctgaacgtgggtttcagtacatttatctgactgaagaagaccatgctcgtattagcacttctgttatagcgcacaagatgcagcttgataatggtgaaattaggtgggttattgattctgttgtggggaaggaggatgggctaggtgtggagaacatacatggaagtgctgctattgccagtgcctattctagggcctatgaggagacatttacgcttacatttgtgactggacggactgttggaataggagcatatcttgctcgacttggcatacggtgcatacagcgtactgaccagcccattatcctaaccgggttctctgctttgaacaagcttcttggccgggaagtgtacagctcccacatgcagttgggtggccccaaaattatggcgacaaacggtgttgtccatctgacagtttcagatgaccttgaaggtgtgtctaatatattgaggtggctcagctatgttcctgccaacattggtggacctcttcctattacaaaatctttggacccacctgacagacccgttgcatatatccctgagaatacatgtgatcctcgtgcagccatcagtggcattgatgatagccaagggaaatggttggggggcatgttcgacaaagacagttttgtggagacatttgaaggatgggcgaagtcagtagttactggcagagcgaaactcggagggattccggtgggtgttatagctgtggagacacagactatgatgcagctcatccctgctgatccaggccagcttgattcccatgagcggtctgttcctcgtgctgggcaagtctggtttccagattcagctactaagacagctcaagcaatgctggacttcaaccgtgaaggattacctctgttcatccttgctaactggagaggcttctctggtgggcaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgaattgagttctatgctgagaggactgcaaagggtaatgttcttgaacctcaagggttgattgagatcaagttcaggtcagaggaactccaagagtgcatgggcaggcttgacccagaattgataaatctgaaggcaaaactcctgggagcaaagcatgaaaatggaagtctatctgagtcagaatcccttcagaagagcatagaagcccggaagaaacagttgttgcctttgtatactcaaattgcggtgcggtttgctgaattgcatgacacttcccttagaatggctgctaagggtgtgattaagaaggttgtagactggaaagattctaggtctttcttctacaagagattacggaggaggatatccgaggacgttcttgcaaaggaaattagaggtgtaagtggcaagcagttctctcaccaatcagcaatcgagctgatccagaaatggtacttggcttctaagggagctgaagcagcaagcactgaatgggatgatgacgatgcttttgttgcctggagggaaaaccctgaaaactaccaggagtatatcaaagaacttagggctcaaagggtatctcagttgctctcagatgttgcagactccagtccagatctagaagccttgccacagggtctttctatgctactagagaagatggatccctcaaggagagcacagtttgttgaggaagtcaagaaagtccttaaatga

SEQ ID NO: 30 - protein, 2311 aa, Triticum aestivum
MGSTHLPIVGFNASTTPSLSTIRPVNSAGAAFQPSAPSRTSKKKSRRVQSLRDGGDGGVSDPNQSIRHGLAGIIDLPKEGTSAPEVDISHGSEEPRGSYQMNGILNEAHNGRHASLSKVVEFCTALGGKTPIHSVLVANNGMAAAKFMRSVRTWANETFGSEKAIQLIAMATPEDMRINAEHIRIADQFVEVPGGTNNNNYANVQLIVEIAVRTGVSAVWPGWGHASENPELPDALNANGIVFLGPPSSSMNALGDKVGSALIAQAAGVPTLPWSGSQVEIPLEVCLDSIPAEMYRKACVSTTEEALASCQMIGYPAMIKASWGGGGKGIRKVNNDDDVRALFKQVQGEVPGSPIFIMRLASQSRHLEVQLLCDQYGNVAALHSRDCSVQRRHQKIIEEGPVTVAPRETVKELEQAARRLAKAVGYVGAATVEYLYSMETGEYYFLELNPRLQVEHPVTEWIAEVNLPAAQVAVGMGIPLWQVPEIRRFYGMDNGGGYDIWRTTAALATPFNFDEVDSQWPKGHCVAVRITSEDPDDGFKPTGGKVKEISFKSKPNVWAYFSVKSGGGIHEFADSQFGHVFAYGVSRAAAITNMSLALKEIQIRGEIHSNVDYTVDLLNASDFKENRIHTGWLDNRIAMRVQAERPPWYISVVGGALYKTITSNTDTVSEYVSYLVKGQIPPKHISLVHSTVSLNIEESKYTIETIRSGQGSYRLRMNGSVIEANVQTLCDGGLLMQLDGNSHVIYAEEEAGGTRLLIDGKTCLLQNDHDPSRLLAETPCKLLRFLVADGAHVEADVPYAEVEVMKMCMPLLSPAAGVINVLLSEGQPMQAGDLIARLDLDDPSAVKRAEPFNGSFPEMSLPIAASGQVHKRCATSLNAARMVLAGYDHPINKVVQDLVSCLDAPELPFLQWEELMSVLATRLPRLLKSELEGKYSEYKLNVGHGKSKDFPSKMLREIIEENLAHGSEKEIATNERLVEPLMSLLKSYEGGRESHAHFIVKSLFEDYLSVEELFSDGIQSDVIERLRQQHSKDLQKVVDIVLSHQGVRNKTKLILTLMEKLVYPNPAAYKDQLTRFSSLNHKRYYKLALKASELLEQTKLSELRTSIARSLSELEMFTEERTAISEIMGDLVTAPLPVEDALVSLFDCSDHTLQQRVIETYISRLYQPHLVKDSIQLKYQESGVIALWEFAEAHSEKRLGAMVIVKSLESVSAAIGAALKDTSRYASSEGNIMHIALLGADNQMHGTEDSGDNDQAQVRIDKLSATLEQNTVTADLCDAGVKVISCIVQRDGALMPMRHTFLLSDEKLCYEEEPVLRHVEPPLSALLELGKLKVKGYNEVKYTPSRDRQWNIYTLRNTENPKMLHRVFFRTLVRQPGASNKFTSGHISDVEVGGAEESLSFTSSSILRSLMTAIEELELHAIRTGHSHMFLCILKEQKLLDLVPVSGNKVVDIGQDEATACSLLKEMALQIHELVGARMHHLSVCQWEVKLKLDSDGPASGTWRVVTTNVTSHTCTVDIYREVEDTESQKLVYHSAPSSSGPLHGVALNTPYQPLSVIDLKRCSARNNRTTYCYDFPLAFETAVQKSWSNISSDNNRCYVKATELVFAQKNGSWGTPVIPMERPAGLNDIGMVAWILDMSTPEYPNGRQIVVIANDITFRAGSFGPREDAFFETVTNLACERKLPLIYLAANSGARIGIADEVKSCFRVGWSDDGSPERGFQYIYLTEEDHARISTSVIAHKMQLDNGEIRWVIDSVVGKEDGLGVENIHGSAAIASAYSRAYEETFTLTFVTGRTVGIGAYLARLGIRCIQRTDQPIILTGFSALNKLLGREVYSSHMQLGGPKIMATNGVVHLTVSDDLEGVSNILRWLSYVPANIGGPLPITKSLDPPDRPVAYIPENTCDPRAAISGIDDSQGKWLGGMFDKDSFVETFEGWAKSVVTGRAKLGGIPVGVIAVETQTMMQLIPADPGQLDSHERSVPRAGQVWFPDSATKTAQAMLDFNREGLPLFILANWRGFSGGQRDLFEGILQAGSTIVENLRTYNQPAFVYIPKAAELRGGAWVVIDSKINPDRIEFYAERTAKGNVLEPQGLIEIKFRSEELQECMGRLDPELINLKAKLLGAKHENGSLSESESLQKSIEARKKQLLPLYTQIAVRFAELHDTSLRMAAKGVIKKVVDWKDSRSFFYKRLRRRISEDVLAKEIRGVSGKQFSHQSAIELIQKWYLASKGAEAASTEWDDDDAFVAWRENPENYQEYIKELRAQRVSQLLSDVADSSPDLEALPQGLSMLLEKMDPSRRAQFVEEVKKVLK

SEQ ID NO: 31 - DNA, 6936 bp, Triticum aestivum
  source 1..6936: mol_type = genomic DNA
atgggatccacacatttgcccattgtcggctttaatgcctcgacgacaccatcactatccactattcgcccggtaaattcagccggtgctgcattccaaccatctgccccttctagaacatccaagaagaaaagtcgtcgtgttcagtcattaagggatggaggcgatggaggcgtgtcagaccctaaccagtctattcgccaaggtcttgccggcatcattgacctcccaaaggagggcacatcagctccggaagtggatatttcacatgggtccgaagaacccagaggctcctaccaaatgaatgggatactgaatgaagcacataatgggaggcatgcttcgctgtctaaggttgtcgaattttgtgtggcattgggcggcaaaacaccaattcacagtgtattagttgcgaacaatggaatggcagcagctaagttcatgcgaagtgtccgaacatgggctaatgaaacatttgggtcagagaaggcaattcagttgatagctatggctactccagaagacatgaggataaatgcagagcacattagaattgctgatcaatttgttgaagtacccggtggaacaaacaataacaactatgcaaatgtccaactcatagtggagatagcagtgagaaccggtgtttctgctgtttggcctggttggggccatgcatctgagaatcctgaacttccagatgcactaaatgcaaacggaattgtttttcttgggccaccatcatcatcaatgaacgcactaggtgacaaggttggttcagctctcattgctcaagcagcaggggttccgactcttccttggagtggatcacaggtggaaattccattagaagtttgtttggactcgatacccgcggagatgtataggaaagcttgtgttagtactacggaggaagcacttgcgagttgtcagatgattgggtatcccgccatgattaaagcatcatggggtggtggtggtaaagggatccgaaaggttaataatgacgatgatgtcagagcactgtttaagcaagtgcaaggtgaagttcctggctccccaatatttatcatgagacttgcatctcagagtcgacatcttgaagttcagttgctttgtgatcaatatggcaatgtagctgcgcttcacagtcgtgactgcagtgtgcaacggcgacaccaaaagattattgaggaaggaccagttactgttgctcctcgcgagacagtgaaagagctagagcaagcagcaaggaggcttgctaaggctgtgggttatgttggtgctgctactgttgaatatctctacagcatggagactggtgaatactattttctggaacttaatccacggttgcaggttgagcatccagtcaccgagtggatagctgaagtaaacttgcctgcagctcaagttgcagttggaatgggtatacccctttggcaggttccagagatcagacgtttctatggaatggacaatggaggaggctatgacatttggaggaaaacagcagctcttgctactccatttaacttcgatgaagtggattctcaatggccaaagggtcattgtgtagcagttaggataaccagtgaggatccagatgacggattcaagcctaccggtggaaaagtaaaggagatcagttttaaaagcaagccaaatgtttgggcttatttctctgttaagtccggtggaggcattcatgaatttgctgattctcagtttggacatgtttttgcatatggagtgtctagagcagcagcaataaccaacatgtctcttgcgctaaaagagattcaaattcgtggagaaattcattcaaatgttgattacacagttgatctcttgaatgcctcagacttcaaagaaaacaggattcatactggctggctggataacagaatagcaatgcgagtccaagctgagagacctccctggtatatttcagtggttggaggagctctatataaaacaataacaagcaacacagacactgtttctgaatatgttagctatctcgtcaagggtcagattccaccaaagcatatatcccttgtccattcaactgtttctttgaatatagaggaaagcaaatatacaattgaaactataaggagcggacagggtagctacagattgcgaatgaatggatcagttattgaagcaaatgtccaaacattatgtgatggtggacttttaatgcagttggatggaaacagccatgtaatttatgctgaagaagaggccggtggtacacggcttctaattgatggaaagacatgcttgttacagaatgatcacgatccttcaaggttattagctgagacaccctgcaaacttcttcgtttcttggttgccgatggtgctcatgttgaagctgatgtaccatatgcggaagttgaggttatgaagatgtgcatgcccctcttgtcacctgctgctggtgtcattaatgttttgttgtctgagggccagcctatgcaggctggtgatcttatagcaagacttgatcttgatgacccttctgctgtgaagagagctgagccatttaacggatctttcccagaaatgagccttcccattgctgcttctggccaagttcacaaaagatgtgccacaagcttgaatgctgctcggatggtccttgcaggatatgatcacccgatcaacaaagttgtacaagatctggtatcctgtctagatgctcctgagcttcctttcctacaatgggaagagcttatgtctgttttagcaaccagacttccaaggcttcttaagagcgagttggagggtaaatacagtgaatataagttaaatgttggccatgggaagagcaaggatttcccttccaagatgctaagagagataatcgaggaaaatcttgcacatggttctgagaaggaaattgctacaaatgagaggcttgttgagcctcttatgagcctactgaagtcatatgagggtggcagagaaagccatgcacactttattgtgaagtcccttttcgaggactatctctcggttgaggaactattcagtgatggcattcagtctgatgtgattgaacgcctgcgccaacaacatagtaaagatctccagaaggttgtagacattgtgttgtctcaccagggtgtgagaaacaaaactaagctgatactaacactcatggagaaactggtctatccaaaccctgctgcctacaaggatcagttgactcgcttttcttccctcaatcacaaaagatattataagttggcccttaaagctagcgagcttcttgaacaaaccaagcttagtgagctccgcacaagcattgcaaggagcctttcagaacttgagatgtttactgaagaaaggacggccattagtgagatcatgggagatttagtgactgccccactgccagttgaagatgcactggtttctttgtttgattgtagtgatcaaactcttcagcagagggtgatcgagacgtacatatctcgattataccagcctcatcttgtcaaggatagtatccagctgaaatatcaggaatctggtgttattgctttatgggaattcgctgaagcgcattcagagaagagattgggtgctatggttattgtgaagtcgttagaatctgtatcagcagcaattggagctgcactaaaggatacatcacgctatgcaagctctgagggtaacataatgcatattgctttattgggtgctgataatcaaatgcatggaactgaagacagtggtgataacgatcaagctcaagtcaggatagacaaactttctgcgacactggaacaaaatactgtcacagctgatctccgtgctgctggtgtgaaggttattagttgcattgttcaaagggatggagcactcatgcctatgcgccataccttcctcttgtcggatgaaaagctttgttatgaggaagagccggttctccggcatgtggagcctcctctttctgctcttcttgagttgggtaagttgaaagtgaaaggatacaatgaggtgaagtatacaccgtcacgtgatcgtcagtggaacatatacacacttagaaatacagaaaaccccaaaatgttgcacagggtgtttttccgaactcttgtcaggcaacccggtgcttccaacaaattcacatcaggccacatcagtgatgttgaagtgggaggagctgaggaatctctttcatttacatcgagcagcatattaagatcgctgatgactgctatagaagagttggagcttcacgcgattcggacaggtcactctcatatgtttttgtgcatattgaaagagcaaaagcttcttgatcttgttcctgtttcagggaacacagttgtggatattggccaagatgaagctactgcatgctcgcttctgaaagaaatggctctacagatacatgaacttgtgggtgcaaggatgcatcatctttctgtatgccaatgggaggtgaaacttaagttggacagcgatgggcctgcgagtggtacctggagagttgtaacaaccaatgttactagtcacacctgcactgtggatatctaccgtgaggttgaagatacagaatcacagaaactagtataccactctgctccatcgtcatctggtcctttgcatggcgttgcactgaatactccatatcagcctttgagtgttattgatctgaaacgttgctccgctagaaacaacagaactacatactgctatgattttccgttggcatttgaaactgcagtgcagaagtcatggtctaacatttctagtgacaataaccgatgttatgttaaagcaacggagctggtgtttgctcacaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcagtacatttatctgactgaagaagaccatgctcgtattagcacttctgttatagcgcacaagatgcagcttgataatggtgaaattaggtgggttatcgattctgttgtggggaaggaggatgggctaggtgtggagaacatacatggaagtgctgctattgccagtgcctattctagggcctatgaggagacatttacgcttacatttgtgactggacggactgttggaataggagcatatcttgctcgacttggcatacggtgcatacagcgtactgaccagcccattatcctaactgggttctctgccttgaacaagcttcttggccgggaagtttacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgcgatcctcgtgctgccatcagtggcattgatgatagccaagggaaatggttggggggcatgttcgacaaagacagttttgtggagacatttgaaggatgggcgaagtcagttgttactggcagagctaaactcggagggattccggtgggtgttatagctgtggagacacagactatgatgcagctcatccctgctgatccaggccagcttgattcccatgagcggtctgttcctcgtgctgggcaagtctggtttccagattcagctactaagacagcgcaggcaatgctggacttcaaccgtgaaggattacctctgttcatccttgctaactggagaggcttctctggtggacaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgcattgagttctatgctgagaggactgcaaagggcaatgttctcgaacctcaagggttgatcgagatcaagttcaggtcagaggaactccaagagtgcatgggtaggcttgatccagaattgataaatctgaaggcaaagctccagggagtaaagcatgaaaatggaagtctacctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtcgttcttctacaagagattacggaggaggatatccgaggatgttcttgcgaaggaaattagaggtgtaagtggcaagcagttttctcaccaatcggcaatcgagctgatccagaaatggtacttggcctctaagggagctgaaacaggaagcactgaatgggatgatgacgatgcttttgttgcctggagggaaaaccctgaaaactaccaggagtatatcaaagaactcagggctcaaagggtatctcagttgctctcagatgttgcagactccagtccagatctagaagccttgccacagggtctttctatgctactagagaagatggatccctcaaggagagcacagtttgttgaggaagtcaagaaagtccttaaatga

SEQ ID NO: 32 - protein, 2311 aa, Triticum aestivum
MGSTHLPIVGFNASTTPSLSTIRPVNSAGAAFQPSAPSRTSKKKSRRVQSLRDGGDGGVSDPNQSIRQGLAGIIDLPKEGTSAPEVDISHGSEEPRGSYQMNGILNEAHNGRHASLSKVVEFCVALGGKTPIHSVLVANNGMAAAKFMRSVRTWANETFGSEKAIQLIAMATPEDMRINAEHIRIADQFVEVPGGTNNNNYANVQLIVEIAVRTGVSAVWPGWGHASENPELPDALNANGIVFLGPPSSSMNALGDKVGSALIAQAAGVPTLPWSGSQVEIPLEVCLDSIPAEMYRKACVSTTEEALASCQMIGYPAMIKASWGGGGKGIRKVNNDDDVRALFKQVQGEVPGSPIFIMRLASQSRHLEVQLLCDQYGNVAALHSRDCSVQRRHQKIIEEGPVTVAPRETVKELEQAARRLAKAVGYVGAATVEYLYSMETGEYYFLELNPRLQVEHPVTEWIAEVNLPAAQVAVGMGIPLWQVPEIRRFYGMDNGGGYDIWRKTAALATPFNFDEVDSQWPKGHCVAVRITSEDPDDGFKPTGGKVKEISFKSKPNVWAYFSVKSGGGIHEFADSQFGHVFAYGVSRAAAITNMSLALKEIQIRGEIHSNVDYTVDLLNASDFKENRIHTGWLDNRIAMRVQAERPPWYISVVGGALYKTITSNTDTVSEYVSYLVKGQIPPKHISLVHSTVSLNIEESKYTIETIRSGQGSYRLRMNGSVIEANVQTLCDGGLLMQLDGNSHVIYAEEEAGGTRLLIDGKTCLLQNDHDPSRLLAETPCKLLRFLVADGAHVEADVPYAEVEVMKMCMPLLSPAAGVINVLLSEGQPMQAGDLIARLDLDDPSAVKRAEPFNGSFPEMSLPIAASGQVHKRCATSLNAARMVLAGYDHPINKVVQDLVSCLDAPELPFLQWEELMSVLATRLPRLLKSELEGKYSEYKLNVGHGKSKDFPSKMLREIIEENLAHGSEKEIATNERLVEPLMSLLKSYEGGRESHAHFIVKSLFEDYLSVEELFSDGIQSDVIERLRQQHSKDLQKVVDIVLSHQGVRNKTKLILTLMEKLVYPNPAAYKDQLTRFSSLNHKRYYKLALKASELLEQTKLSELRTSIARSLSELEMFTEERTAISEIMGDLVTAPLPVEDALVSLFDCSDQTLQQRVIETYISRLYQPHLVKDSIQLKYQESGVIALWEFAEAHSEKRLGAMVIVKSLESVSAAIGAALKDTSRYASSEGNIMHIALLGADNQMHGTEDSGDNDQAQVRIDKLSATLEQNTVTADLRAAGVKVISCIVQRDGALMPMRHTFLLSDEKLCYEEEPVLRHVEPPLSALLELGKLKVKGYNEVKYTPSRDRQWNIYTLRNTENPKMLHRVFFRTLVRQPGASNKFTSGHISDVEVGGAEESLSFTSSSILRSLMTAIEELELHAIRTGHSHMFLCILKEQKLLDLVPVSGNTVVDIGQDEATACSLLKEMALQIHELVGARMHHLSVCQWEVKLKLDSDGPASGTWRVVTTNVTSHTCTVDIYREVEDTESQKLVYHSAPSSSGPLHGVALNTPYQPLSVIDLKRCSARNNRTTYCYDFPLAFETAVQKSWSNISSDNNRCYVKATELVFAHKNGSWGTPVIPMERPAGLNDIGMVAWILDMSTPEYPNGRQIVVIANDITFRAGSFGPREDAFFETVTNLACERKLPLIYLAANSGARIGIADEVKSCFRVGWSDDGSPERGFQYIYLTEEDHARISTSVIAHKMQLDNGEIRWVIDSVVGKEDGLGVENIHGSAAIASAYSRAYEETFTLTFVTGRTVGIGAYLARLGIRCIQRTDQPIILTGFSALNKLLGREVYSSHMQLGGPKIMATNGVVHLTVSDDLEGVSNILRWLSYVPANIGGPLPITKSLDPPDRPVAYIPENTCDPRAAISGIDDSQGKWLGGMFDKDSFVETFEGWAKSVVTGRAKLGGIPVGVIAVETQTMMQLIPADPGQLDSHERSVPRAGQVWFPDSATKTAQAMLDFNREGLPLFILANWRGFSGGQRDLFEGILQAGSTIVENLRTYNQPAFVYIPKAAELRGGAWVVIDSKINPDRIEFYAERTAKGNVLEPQGLIEIKFRSEELQECMGRLDPELINLKAKLQGVKHENGSLPESESLQKSIEARKKQLLPLYTQIAVRFAELHDTSLRMAAKGVIKKVVDWEDSRSFFYKRLRRRISEDVLAKEIRGVSGKQFSHQSAIELIQKWYLASKGAETGSTEWDDDDAFVAWRENPENYQEYIKELRAQRVSQLLSDVADSSPDLEALPQGLSMLLEKMDPSRRAQFVEEVKKVLK

SEQ ID NO: 33 - DNA, 6936 bp, Triticum aestivum
  source 1..6936: mol_type = genomic DNA
atgggatccacacatttgcccattgtcggccttaatgcctcgacaacaccatcgctatccactattcgcccggtaaattcagccggtgctgcattccaaccatctgccccttctagaacctccaagaagaaaagtcgtcgtgttcagtcattaagggatggaggcgatggaggcgtgtcagaccctaaccagtctattcgccaaggtcttgccggcatcattgacctcccaaaggagggcacatcagctccggaagtggatatttcacatgggtccgaagaacccaggggctcctaccaaatgaatgggatactgaatgaagcacataatgggaggcatgcttcgctgtctaaggttgtcgaattttgtatggcattgggcggcaaaacaccaattcacagtgtattagttgcgaacaatggaatggcagcagctaagttcatgcggagtgtccgaacatgggctaatgaaacatttgggtcagagaaggcaattcagttgatagctatggctactccagaagacatgaggataaatgcagagcacattagaattgctgatcaatttgttgaagtacccggtggaacaaacaataacaactatgcaaatgtccaactcatagtggagatagcagtgagaaccggtgtttctgctgtttggcctggttggggccatgcatctgagaatcctgaacttccagatgcactaaatgcaaacggaattgtttttcttgggccaccatcatcatcaatgaacgcactaggtgacaaggttggttcagctctcattgctcaagcagcaggggttccgactcttccttggagtggatcacaggtggaaattccattagaagtttgtttggactcgatacctgcggagatgtataggaaagcttgtgttagtactacggaggaagcacttgcgagttgtcagatgattgggtatccagccatgattaaagcatcatggggtggtggtggtaaagggatccgaaaggttaataacgacgatgatgtcagagcactgtttaagcaagtgcaaggtgaagttcctggctccccaatatttatcatgagacttgcatctcagagtcgacatcttgaagttcagttgctttgtgatcaatatggcaatgtagctgcgcttcacagtcgtgactgcagtgtgcaacggcgacaccaaaagattattgaggaaggaccagttactgttgctcctcgcgagacagtgaaagagctagagcaagcagcaaggaggcttgctaaggctgtgggttatgttggtgctgctactgttgaatatctctacagcatggagactggtgaatactattttctggaacttaatccacggttgcaggttgagcatccagtcaccgagtggatagctgaagtaaacttgcctgcagctcaagttgcagttggaatgggtatacccctttggcaggttccagagatcagacgtttctatggaatggacaatggaggaggctatgacatttggaggaaaacagcagctcttgctaccccatttaactttgatgaagtggattctcaatggccaaagggtcattgtgtagcagttaggataaccagtgaggatccagatgacggattcaagcctaccggtggaaaagtaaaggagatcagttttaaaagcaagccaaatgtttgggcctatttctctgttaagtccggtggaggcattcatgaatttgctgattctcagtttggacatgtttttgcatatggagtgtctagagcagcagcaataaccaacatgtctcttgcgctaaaagagattcaaattcgtggagaaattcattcaaatgttgattacacagttgatctcttgaatgcctcagacttcaaagaaaacaggattcatactggctggctggataacagaatagcaatgcgagtccaagctgagagacctccgtggtatatttcagtggttggaggagctctatataaaacaataacgagcaacacagacactgtttctgaatatgttagctatctcgtcaagggtcagattccaccgaagcatatatcccttgtccattcaactgtttctttgaatatagaggaaagcaaatatacaattgaaactataaggagcggacagggtagctacagattgcgaatgaatggatcagttattgaagcaaatgtccaaacattatgtgatggtggacttttaatgcagttggatggaaacagccatgtaatttatgctgaagaagaggccggtggtacacggcttctaattgatggaaagacatgcttgttacagaatgatcacgatccttcaaggttattagctgagacaccctgcaaacttcttcgtttcttggttgccgatggtgctcatgttgaagctgatgtaccatatgcggaagttgaggttatgaagatgtgcatgcccctcttgtcacctgctgctggtgtcattaatgttttgttgtctgagggccagcctatgcaggctggtgatcttatagcaagacttgatcttgatgacccttctgctgtgaagagagctgagccatttaacggatctttcccagaaatgagccttcctattgctgcttctggccaagttcacaaaagatgtgccacaagcttgaatgctgctcggatggtccttgcaggatatgatcacccgatcaacaaagttgtacaagatctggtatcctgtctagatgctcctgagcttcctttcctacaatgggaagagcttatgtctgttttagcaactagacttccaaggcttcttaagagcgagttggagggtaaatacagtgaatataagttaaatgttggccatgggaagagcaaggatttcccttccaagatgctaagagagataatcgaggaaaatcttgcacatggttctgagaaggaaattgctacaaatgagaggcttgttgagcctcttatgagcctactgaagtcatatgagggtggcagagaaagccatgcacactttattgtgaagtcccttttcgaggactatctctcggttgaggaactattcagtgatggcattcagtctgatgtgattgaacgcctgcgccaacaacatagtaaagatctccagaaggttgtagacattgtgttgtctcaccagggtgtgagaaacaaaactaagctgatactaacactcatggagaaactggtctatccaaaccctgctgcctacaaggatcagttgactcgcttttcctccctcaatcacaaaagatattataagttggcccttaaagctagcgagcttcttgaacaaaccaagcttagtgagctccgcacaagcattgcaaggagcctttcagaacttgagatgtttactgaagaaaggacggccattagtgagatcatgggagatttagtgactgccccactgccagttgaagatgcactggtttctttgtttgattgtagtgatcaaactcttcagcagagggtgatcgagacgtacatatctcgattataccagcctcatcttgtcaaggatagtatccagctgaaatatcaggaatctggtgttattgctttatgggaattcgctgaagcgcattcagagaagagattgggtgctatggttattgtgaagtcgttagaatctgtatcagcagcaattggagctgcactaaagggtacatcacgctatgcaagctctgagggtaacataatgcatattgctttattgggtgctgataatcaaatgcatggaactgaagacagtggtgataacgatcaagctcaagtcaggatagacaaactttctgcgacactggaacaaaatactgtcacagctgatctccgtgctgctggtgtgaaggttattagttgcattgttcaaagggatggagcactcatgcctatgcgccataccttcctcttgtcggatgaaaagctttgttatgaggaagagccggttctccggcatgtggagcctcctctttctgctcttcttgagttgggtaagttgaaagtgaaaggatacaatgaggtgaagtatacaccgtcacgtgatcgtcagtggaacatatacacacttagaaatacagagaaccccaaaatgttgcacagggtgtttttccgaactcttgtcaggcaacccggtgcttccaacaaattcacatcaggcaacatcagtgatgttgaagtgggaggagctgaggaatctctttcatttacatcgagcagcatattaagatcgctgatgactgctatagaagagttggagcttcacgcgattaggacaggtcactctcatatgtttttgtgcatattgaaagagcaaaagcttcttgatcttgttcccgtttcagggaacaaagttgtggatattggccaagatgaagctactgcatgcttgcttctgaaagaaatggctctacagatacatgaacttgtgggtgcaaggatgcatcatctttctgtatgccaatgggaggtgaaacttaagttggacagcgatgggcctgccagtggtacctggagagttgtaacaaccaatgttactagtcacacctgcactgtggatatctaccgtgaggtcgaagatacagaatcacagaaactagtgtaccactctgctccatcgtcatctggtcctttgcatggcgttgcactgaatactccatatcagcctttgagtgttattgatctgaaacgttgctccgctagaaataacagaactacatactgctatgattttccgttggcatttgaaactgcagtgcagaagtcatggtctaacatttctagtgacactaaccgatgttatgttaaagcgacggagctggtgtttgctcacaagaacgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcaatatatttatctgactgaagaagaccatgctcgtattagcgcttctgttatagcgcacaagatgcagcttgataatggtgaaattaggtgggttattgattctgttgtagggaaggaggatgggctaggtgtggagaacatacatggaagtgctgctattgccagtgcctattctagggcctatgaggagacatttacgcttacatttgtgactggaaggactgttggaataggagcatatcttgctcgacttggcatacggtgcattcagcgtactgaccagcccattatcctaactgggttttctgccttgaacaagcttcttggccgggaagtgtacagctcccacatgcagttgggtggccccaaaattatggccacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgtgatcctcgtgcagccatcagtggcattgatgatagccaagggaaatggttggggggcatgttcgacaaagacagttttgtggagacatttgaaggatgggcgaagtcagtagttactggcagagcgaaactcggagggattccggtgggtgttatagctgtggagacacagactatgatgcagctcatccctgctgatccaggtcagcttgattcccatgagcggtctgttcctcgtgctgggcaagtctggtttccagattcagctactaagacagcgcaggcaatgctggacttcaaccgtgaaggattacctctgttcatccttgctaactggagaggcttctctggtgggcaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgcattgagttctatgctgagaggactgcaaagggcaatgttcttgaacctcaagggttgattgagatcaagttcaggtcagaggaactccaagagtgcatgggcaggcttgacccagaattgataaatttgaaggcaaaactcctgggagcaaagcatgaaaatggaagtctatctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtctttcttctacaagagattacggaggaggatatccgaggatgttcttgcaaaggaaattagaggtgtaagtggcaagcagttttctcaccaatcggcaatcgagctgatccagaaatggtacttggcctctaagggagctgaaacaggaaacactgaatgggatgatgacgatgcttttgttgcctggagggaaaaccctgaaaactaccaggagtatatcaaagaactcagggctcaaagggtatctcagttgctctcagatgttgcagactccagtccagatctagaagccttgccacagggtctttctatgctactagagaagatggatccctcaaggagagcacagtttgttgaggaagtcaagaaggtccttaaatga

SEQ ID NO: 34 - protein, 2311 aa, Triticum aestivum
MGSTHLPIVGLNASTTPSLSTIRPVNSAGAAFQPSAPSRTSKKKSRRVQSLRDGGDGGVSDPNQSIRQGLAGIIDLPKEGTSAPEVDISHGSEEPRGSYQMNGILNEAHNGRHASLSKVVEFCMALGGKTPIHSVLVANNGMAAAKFMRSVRTWANETFGSEKAIQLIAMATPEDMRINAEHIRIADQFVEVPGGTNNNNYANVQLIVEIAVRTGVSAVWPGWGHASENPELPDALNANGIVFLGPPSSSMNALGDKVGSALIAQAAGVPTLPWSGSQVEIPLEVCLDSIPAEMYRKACVSTTEEALASCQMIGYPAMIKASWGGGGKGIRKVNNDDDVRALFKQVQGEVPGSPIFIMRLASQSRHLEVQLLCDQYGNVAALHSRDCSVQRRHQKIIEEGPVTVAPRETVKELEQAARRLAKAVGYVGAATVEYLYSMETGEYYFLELNPRLQVEHPVTEWIAEVNLPAAQVAVGMGIPLWQVPEIRRFYGMDNGGGYDIWRKTAALATPFNFDEVDSQWPKGHCVAVRITSEDPDDGFKPTGGKVKEISFKSKPNVWAYFSVKSGGGIHEFADSQFGHVFAYGVSRAAAITNMSLALKEIQIRGEIHSNVDYTVDLLNASDFKENRIHTGWLDNRIAMRVQAERPPWYISVVGGALYKTITSNTDTVSEYVSYLVKGQIPPKHISLVHSTVSLNIEESKYTIETIRSGQGSYRLRMNGSVIEANVQTLCDGGLLMQLDGNSHVIYAEEEAGGTRLLIDGKTCLLQNDHDPSRLLAETPCKLLRFLVADGAHVEADVPYAEVEVMKMCMPLLSPAAGVINVLLSEGQPMQAGDLIARLDLDDPSAVKRAEPFNGSFPEMSLPIAASGQVHKRCATSLNAARMVLAGYDHPINKVVQDLVSCLDAPELPFLQWEELMSVLATRLPRLLKSELEGKYSEYKLNVGHGKSKDFPSKMLREIIEENLAHGSEKEIATNERLVEPLMSLLKSYEGGRESHAHFIVKSLFEDYLSVEELFSDGIQSDVIERLRQQHSKDLQKVVDIVLSHQGVRNKTKLILTLMEKLVYPNPAAYKDQLTRFSSLNHKRYYKLALKASELLEQTKLSELRTSIARSLSELEMFTEERTAISEIMGDLVTAPLPVEDALVSLFDCSDQTLQQRVIETYISRLYQPHLVKDSIQLKYQESGVIALWEFAEAHSEKRLGAMVIVKSLESVSAAIGAALKGTSRYASSEGNIMHIALLGADNQMHGTEDSGDNDQAQVRIDKLSATLEQNTVTADLRAAGVKVISCIVQRDGALMPMRHTFLLSDEKLCYEEEPVLRHVEPPLSALLELGKLKVKGYNEVKYTPSRDRQWNIYTLRNTENPKMLHRVFFRTLVRQPGASNKFTSGNISDVEVGGAEESLSFTSSSILRSLMTAIEELELHAIRTGHSHMFLCILKEQKLLDLVPVSGNKVVDIGQDEATACLLLKEMALQIHELVGARMHHLSVCQWEVKLKLDSDGPASGTWRVVTTNVTSHTCTVDIYREVEDTESQKLVYHSAPSSSGPLHGVALNTPYQPLSVIDLKRCSARNNRTTYCYDFPLAFETAVQKSWSNISSDTNRCYVKATELVFAHKNGSWGTPVIPMERPAGLNDIGMVAWILDMSTPEYPNGRQIVVIANDITFRAGSFGPREDAFFETVTNLACERKLPLIYLAANSGARIGIADEVKSCFRVGWSDDGSPERGFQYIYLTEEDHARISASVIAHKMQLDNGEIRWVIDSVVGKEDGLGVENIHGSAAIASAYSRAYEETFTLTFVTGRTVGIGAYLARLGIRCIQRTDQPIILTGFSALNKLLGREVYSSHMQLGGPKIMATNGVVHLTVSDDLEGVSNILRWLSYVPANIGGPLPITKSLDPPDRPVAYIPENTCDPRAAISGIDDSQGKWLGGMFDKDSFVETFEGWAKSVVTGRAKLGGIPVGVIAVETQTMMQLIPADPGQLDSHERSVPRAGQVWFPDSATKTAQAMLDFNREGLPLFILANWRGFSGGQRDLFEGILQAGSTIVENLRTYNQPAFVYIPKAAELRGGAWVVIDSKINPDRIEFYAERTAKGNVLEPQGLIEIKFRSEELQECMGRLDPELINLKAKLLGAKHENGSLSESESLQKSIEARKKQLLPLYTQIAVRFAELHDTSLRMAAKGVIKKVVDWEDSRSFFYKRLRRRISEDVLAKEIRGVSGKQFSHQSAIELIQKWYLASKGAETGNTEWDDDDAFVAWRENPENYQEYIKELRAQRVSQLLSDVADSSPDLEALPQGLSMLLEKMDPSRRAQFVEEVKKVLK

SEQ ID NO: 35 - DNA, 1944 bp, Triticum aestivum
  source 1..1944: mol_type = genomic DNA
atggccgccgccacctcccccgccgtcgcattctccggcgccgccgccgccgccgccgccatgcccaagcccgcccgccagcctctcccgcgccaccagcccgcctcgcgccgcgcgctccccgcccgcgtcgtcaggtgctgcgccgcgccccccgctgctgccacctccgccgcgccccccgccaccgcgctccggccctggggcccgtccgagccccgcaagggcgccgacatcctcgtcgaggcgctcgagcgctgcggcatcgtcgacgtattcgcctaccccggcggcgcgtccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgctgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttcctcgcgtcctctggccgcccggggccggtgctggttgatatccccaaggatatccagcagcagatggccgtgcctatctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatccggcgaggagttgcgccgctttgttgagctcactgggattccggttacaactactctgatgggccttggcaacttccccagcgacgacccactgtctctgcgcatgcttgggatgcatggcactgtgtatgcaaattatgcagtcgataaggctgacctgttgcttgcatttggtgtgcggtttgatgatcgcgtgactgggaaaatcgaggcctttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgctactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggagatggtagtttcctcatgaacattcaggagttggcattgatccgtattgagaacctccctgtgaaggtgatgatattgaacaaccagcatctgggaatggtggtgcaatgggaggataggttttacaaggccaatcgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcatcgtcccgcatcaggagcacgtgctgcctatgatcccaagcggtggtgctttcaaggacatgatcatggagggtgatggcaggacctcgtactga

SEQ ID NO: 36 - protein, 647 aa, Triticum aestivum
MAAATSPAVAFSGAAAAAAAMPKPARQPLPRHQPASRRALPARVVRCCAAPPAAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPIWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 37 - DNA, 1935 bp, Triticum aestivum
  source 1..1935: mol_type = genomic DNA
atggccgcagccacctcccccgccgtcgcattctcgggcgccgccgccgccgccatacccaaacccgcccgccagcctctcccgcgccaccagcccgcctcgcgccgcgcgctccccgcccgcatcgtcaggtgctgcgccgcgtcccccgccgccacctccgtcgcgcctcccgccaccgcgctccggccgtggggcccctccgagccccgtaagggcgccgacatcctcgtcgaggcgctggagcgctgcggcatcgtcgacgtcttcgcctaccctggcggcgcgtccatggagatccaccaggcgctgacgcgctcgccagtcatcaccaaccacctcttccgccacgagcagggggaggcgttcgcggcgtccgggtacgcccgcgcgtccggccgcgtcggcgtctgcgtcgccacctccggcccgggggccaccaacctcgtctccgcgctcgccgacgctctcctcgactccatccccatggtcgccatcacgggccaggtcccccgccgcatgatcggcacggatgcgttccaggagacgcccatcgtggaggtcacgcgctccatcaccaagcacaactacctggtccttgacgtggaggatatcccccgcgtcatccaggaagccttcttcctcgcatcctctggccgcccggggccggtgctggttgatatccccaaggacatccagcagcagatggctgtgcctgtctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatctggtgaggagttgcgccgctttgttgagctcactgggattccagttacaactactcttatgggccttggcaacttccccagtgacgacccactgtctctgcgcatgctggggatgcatggcactgtgtatgcaaattatgcagtagataaggctgacctgttgcttgcatttggtgtgcggtttgatgatcgtgtgaccgggaaaatcgaggcttttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggtgaggccatcccgccgcaatatgctatccaggtactggatgagctgacaaaaggggaggcgatcattgccaccggtgttgggcagcatcagatgtgggcggctcagtattacacttacaagcggccacggcagtggctgtcttcgtccggtttgggtgcaatgggatttgggttgccagctgcagctggcgctgctgtggccaacccaggtgttacagttgttgacattgatggggatggtagtttcctcatgaacattcaggagttggcgttgatccgtattgagaacctcccagtgaaggtgatgatattgaacaaccagcatctgggaatggtggtgcagtgggaggataggttttacaaggccaaccgggcgcacacataccttggcaacccagaaaatgagggtgagatatatccagattttgtgacgattgctaaaggattcaacgttccggcagttcgtgtgacgaagaagagcgaagtcactgcagcaatcaagaagatgcttgagaccccagggccatacttgttggatatcattgtcccgcatcaggagcacgtgctgcctatgatcccaagcggtggtgcttttaaggacatgatcatggagggtgatggcaggacctcgtactga

SEQ ID NO: 38 - protein, 644 aa, Triticum aestivum
MAAATSPAVAFSGAAAAAIPKPARQPLPRHQPASRRALPARIVRCCAASPAATSVAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENEGEIYPDFVTIAKGFNVPAVRVTKKSEVTAAIKKMLETPGPYLLDIIVPHQEHVLPMIPSGGAFKDMIMEGDGRTSY

SEQ ID NO: 39 - DNA, 1935 bp, Triticum aestivum
  source 1..1935: mol_type = genomic DNA
atggccgccgccacctcccccgccgtcgcattctcgggcgccaccgccgccgccatgcccaaacccgcccgccatcctctcccgcgccaccagcccgtctcgcgccgcgcgctccccgcccgcgtcgtcaggtgttgcgccgcgtcccccgccgccacctccgccgcgcctcccgcaaccgcgctccggccctggggcccgtccgagccccgcaagggcgccgacatcctcgtcgaggcgctcgagcgctgcggcatcgtcgacgtcttcgcctaccccggcggcgcctccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgccgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttccttgcatcctctggccgcccggggccggtgctagttgatatccccaaggacatccagcagcagatggctgtgcccgtctgggacactccaatgagtttgccagggtacatcgcccgcctgcccaagccaccatctactgaatcgcttgagcaggtcctgcgtctggttggcgagtcacggcgcccaattctgtatgttggtggtggctgcgctgcgtctggcgaggagttgcgccgctttgttgagcttactgggattccagttacaactactctgatgggccttggcaacttccccagcgacgacccactgtctctgcgcatgcttgggatgcatggcactgtgtatgcaaattatgcagtagataaggctgacctgttgctcgcatttggtgtgcggtttgatgatcgtgtgactgggaaaatcgaggcttttgcaagcaggtccaagattgtgcacattgacattgacccagctgagattggcaagaacaagcagccacatgtctccatttgtgcagatgttaagcttgctttacaggggttgaatgatctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctgggaatggtggtgcagtgggaggataggttttacaaggccaatcgggcgcacacataccttggcaacccagaaaatgagagtgagatatatccagattttgtgacgattgctaaaggattcaacgttccagcagttcgagtgacgaagaagagcgaagtcactgcagcaatcaagaagatgcttgagaccccagggccatacttgttggatatcatagtcccgcatcaggagcacgtgctgcctatgatcccaagcggtggtgctttcaaggacatgatcatggagggtgatggcaggacctcgtactga

SEQ ID NO: 40 - protein, 644 aa, Triticum aestivum
MAAATSPAVAFSGATAAAMPKPARHPLPRHQPVSRRALPARVVRCCAASPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 41 - DNA, 1551 bp, Triticum aestivum
  source 1..1551: mol_type = genomic DNA
atggagaaggcatacattgccatcctctccttcgccttcctcttcctgctccactacattctaggcaaggtcagcaatggcaggcgcagcaagggccccgtccatctgccgccgagcccccgggccatcccattcctcggccacctccacctcctggagaagccgttccacgccgctctgtgcggcctcgccaagcgcctcggcccggtcttctcgctgcgcctcggctcgcgccgcgccgtggtcgtctcctcggcggagtgcgccagggagtgcttcacggagcacgacgtgatcttcgccaaccggccccagttcccctcgcagctgctcgtgtccttcgacggcatcgcgctctccacgtccagctacggcccgcactggcgcaacctccgccgcgtcgccgccgtgcagctgctctctgcgcaccgcgtcgcctgcatgtcgggcgtgatcgagggcgaggtgcgcgccatggcgcgccggcttttccgggcctccgtggcgtcccccggcggcgccgcgcgggtcgagctgaagcggaggctctttgagctctccctcagcgtgctcatggagaccatcgcccagaccaaggggacccggtcggaggccgacgccgacacggacatgtccgtggaggcccaggagttcaagaaggtggtggacgagatcatcccgcacatcggcgccgccaacctgtgggactacctgccagtgctgcggtggttcgacgtgttcggagtgaggaacaagatccttgccgcggtgagcaggagggacgccttcatgctgcgtctcatcgacgccgagcgccggaggcttgaagacggcggcgccgaaggcgacaagaagagcatgatcgccgtgttgctcactctgcagaagacggagccggaggtgtacaccgatactatgatcacggctctctgtgcgaatttgtttggggctgggacggagaccacgtcgacgacgacggagtgggcgatgtcgctgctgctgaaccacccggcggcgctgaggaaggcgcaggccgagattgatgtggccgtggggacctcccggctgttgaccgccgacgacgtgccgcggctcgcctacctgcagtgcatcgtgagcgagacgctgcggctgtacccggcggcgccgatgctgctgccgcaccagtcgtccgcggactgcaaggtgggcggctacaacgtgccgagcggcacgatgctgatggtgaacgcgtacgccatccaccgggacccggcggcgtgggagcggccgctggagttcgtcccggagcggttcgaggacgggaaggcggaggggcggttcatgatcccgttcgggatgggccggcggcggtgccccggggagacgctggcgctgcggaccatcggcatggtgctggccacgctggtgcagtgcttcgactgggagcgcgtcgacggcgcggaggtggacatgacggagggcggcgggctcaccatccccaaggccgtgcctctcgaggccgtgtgcaggccgcgcccggccatgcgcgacgtgcttcagagcctctga

SEQ ID NO: 42 - protein, 516 aa, Triticum aestivum
MEKAYIAILSFAFLFLLHYILGKVSNGRRSKGPVHLPPSPRAIPFLGHLHLLEKPFHAALCGLAKRLGPVFSLRLGSRRAVVVSSAECARECFTEHDVIFANRPQFPSQLLVSFDGIALSTSSYGPHWRNLRRVAAVQLLSAHRVACMSGVIEGEVRAMARRLFRASVASPGGAARVELKRRLFELSLSVLMETIAQTKGTRSEADADTDMSVEAQEFKKVVDEIIPHIGAANLWDYLPVLRWFDVFGVRNKILAAVSRRDAFMLRLIDAERRRLEDGGAEGDKKSMIAVLLTLQKTEPEVYTDTMITALCANLFGAGTETTSTTTEWAMSLLLNHPAALRKAQAEIDVAVGTSRLLTADDVPRLAYLQCIVSETLRLYPAAPMLLPHQSSADCKVGGYNVPSGTMLMVNAYAIHRDPAAWERPLEFVPERFEDGKAEGRFMIPFGMGRRRCPGETLALRTIGMVLATLVQCFDWERVDGAEVDMTEGGGLTIPKAVPLEAVCRPRPAMRDVLQSL

SEQ ID NO: 43 - DNA, 3602 bp, Zea mays
  source 1..3602: mol_type = genomic DNA
aacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatgcagatcttcgtcaagactctcactggaaagactatcaccctcgaggtggagtcctctgacaccattgacaacgtcaaggccaagatccaggacaaggaaggcattcccccggaccagcagcggctcatcttcgctggcaagcagctcgaggacgggcgcacacttgccgactacaacatccagaaggagagcaccctccaccttgttctccgcctcaggggaggcatgcagatcttcgtcaagaccctcactggaaagactatcaccctcgaggtggagtcttctgacaccatcgacaacgtcaaggccaagatccaggacaaggagggcatccctccggaccagcagcggctcatttttgctggcaagcagctcgaggacgggcgcacgctggccgactacaacattcagaaggagagcaccctccacttggtgttgcgtctcaggggcggcatgcagatcttcgtgaagactctgactggcaagactatcacccttgaggtcgagtcttctgataccattgacaatgtcaaggccaagatccaggacaaggagggcatccctccggaccagcagcggctcatttttgccggcaagcagctcgaggacgggcgcacgcttgccgactacaacattcagaaggagagcaccctccacttggtgctgcgtctcaggggtggcatgcagatcttcgtgaagacacttactggcaagacgatcacccttgaggtggagtcctcggacaccatcgacaatgtcaaggccaagatccaggacaaggagggcattccaccggaccagcagcggctcatctttgctggcaagcagctggaggatggccgtacccttgcagattacaacatccagaaggagagcaccctccacttggtgctgcgtctcaggggtggcatgcagatcttcgtgaagactcttactggcaagacgatcacccttgaggtggagtcctcagacaccattgacaacgtcaaggccaagatccaggacaaggagggcattccaccggaccagcagcggctgatcttcgccggcaagcagctggaggatggtcgcaccttggctgactacaacatccagaaggagagcaccctccacttggtgctccgcctcaggggtggcatgcagatctttgtgaagacattgactggcaagaccatcaccttggaggtggagtcgtctgacacaatcgataatgtcaaagccaagatccaggacaaggagggcattccaccggaccagcagcgcctgatctttgctggcaagcagctggaggatggccgtaccctcgccgactacaacatccagaaggagagcaccctccacctggtgctccgcttgcgaggtggtatgcagatctttgtgaagaccttgactggcaagaccataaccctggaggttgagagctccgacaccatcgacaacgtgaaggcgaagatccaggacaaagagggcatccccccagaccagcagcgtctgatcttcgccggcaagcagctggaggatggccgcaccctagcagactacaacatccagaaggagagcactctccacctggtgctccgcctccgcggtggtcagtaagccatggttcatttaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattatg

SEQ ID NO: 44 - DNA, 3602 bp, Zea mays
  source 1..3602: mol_type = genomic DNA
tcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatgcagatctttgtgaaaaccctgactggcaagactatcaccctcgaggtggagtcgtctgacaccattgacaacgttaaggccaagatccaggacaaggagggcatccccccagaccagcagcggctcatctttgctggcaaacagcttgaggacgggcgcacgcttgctgactacaacatccagaaggagagcaccctccaccttgtgctccgtctcaggggaggcatgcagatctttgtgaaaaccctgaccggcaagactatcaccctcgaggtggagtcctctgacaccattgacaacgtcaaggccaagatccaggacaaggagggcatccctccagaccagcagcggctcatctttgctgggaagcagcttgaggacgggcgcacgcttgccgactacaacatccagaaggagagcaccctccacttggtgctgcgcctcaggggaggcatgcagatcttcgtgaagaccctgaccggcaagactatcaccctcgaggtggagtcttcagacaccatcgacaacgtcaaggccaagatccaggacaaggagggcattcccccagaccagcagcggctcatctttgctggaaagcagcttgaggacgggcgcacgcttgccgactacaacatccagaaggagagcaccctccacttggtgctgcgcctcaggggaggcatgcagatcttcgtgaagaccctgaccggcaagactatcaccctcgaggtggagtcttcagacaccatcgacaatgtcaaggccaagatccaggacaaggagggcatcccaccggaccagcagcgtttgatcttcgctggcaagcagctggaggatggccgcacccttgcggattacaacatccagaaggagagcaccctccacctggtgctccgtctcaggggtggtatgcagatctttgtgaagacactcactggcaagacaatcacccttgaggtggagtcttcggataccattgacaatgtcaaggccaagatccaggacaaggagggcatcccacccgaccagcagcgcctcatcttcgccggcaagcagctggaggatggccgcaccctggcggattacaacatccagaaggagagcactctccacctggtgctccgcctcaggggtggcatgcagatttttgtgaagacattgactggcaagaccatcaccttggaggtggagagctctgacaccattgacaatgtgaaggccaagatccaggacaaggagggcattcccccagaccagcagcgtctgatctttgcgggcaagcagctggaggatggccgcactctcgcggactacaacatccagaaggagagcacccttcaccttgttctccgcctcagaggtggtatgcagatctttgtaaagaccctgactggaaaaaccataaccctggaggttgagagctcggacaccatcgacaatgtgaaggcgaagatccaggacaaggagggcatccccccggaccagcagcgtctgatcttcgccggcaaacagctggaggatggccgcaccctagcagactacaacatccaaaaggagagcaccctccaccttgtgctccgtctccgtggtggtcagtaagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggatt

SEQ ID NO: 45 - DNA, 8385 bp, Zea mays
  source 1..8385: mol_type = genomic DNA
tccccgtggtttgtctcccagctcctctcttgatttgctaggctcttgtctattatctcacatctcccctccccttgatctgctaggtgcttgatggattgaagtctgccatccccacggtcgcaggcgctaatgagggcttgacgattttctggaagcaaccggaaaaaagctaggcgggaatgacgtgcaaagtgggagagaaacagagaggactacgtacaggaacatgaggtagttgcaaacgtaagccattttagggtccatgattggaattgacaattgaatttgaaccataaaacaaatagaataggtagtaaattaaattatagagcctaactataaggcttatggttggagttgctctaaggacgacagaaaagggacatttagagagttaaacacaagctagaagtaataagattatcctaattaagtttcacacataaacacactatctaatttatcaatgttgtatgttttgttttacccaaaactgtgtgtaataaaaacagattgggattgatacaacatcatgaattgtaagacaaacattatatacggttacacataaacacactgtctaattcatcaatgatgtatgttacatagaatgagattggaaaaccaaaacaacgaataagcaaactgcaggaaaagtatgcagtggaaaccaacccagattcggacgataggaaagtatcatgtgaatgatttgccaggaaaaggagaggggtaaaaaggggcgaagatttagaagatctaaagcacaagaaccagagattagattgaacaatagggaacatggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaaggtataaagaatacattaacattcagtgaaacagcatgctgtcttcttctttttttatgcacaacagagcatacatatataccttcccaggctgaggacttggcggaggagagccgcggagaggatggcggtgcagacggtctggacgggcccgaagacggagacgaacagcgggcccttcctgcccaggcaccacgcttggaacgccatgcacgcgccaaccgcggccccgccgaggacgacgatccccgcgacaagcgtggcgtcgatcctgggcgaccccatgccgaggaacctcccttccaggacgagccgcaggacggcggtgagagaggcacccgtcgcggaggtggcgcagcacatggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatgcagatcttcgtcaagactctcactggaaagactatcaccctcgaggtggagtcctctgacaccattgacaacgtcaaggccaagatccaggacaaggaaggcattcccccggaccagcagcggctcatcttcgctggcaagcagctcgaggacgggcgcacacttgccgactacaacatccagaaggagagcaccctccaccttgttctccgcctcaggggaggcatgcagatcttcgtcaagaccctcactggaaagactatcaccctcgaggtggagtcttctgacaccatcgacaacgtcaaggccaagatccaggacaaggagggcatccctccggaccagcagcggctcatttttgctggcaagcagctcgaggacgggcgcacgctggccgactacaacattcagaaggagagcaccctccacttggtgttgcgtctcaggggcggcatgcagatcttcgtgaagactctgactggcaagactatcacccttgaggtcgagtcttctgataccattgacaatgtcaaggccaagatccaggacaaggagggcatccctccggaccagcagcggctcatttttgccggcaagcagctcgaggacgggcgcacgcttgccgactacaacattcagaaggagagcaccctccacttggtgctgcgtctcaggggtggcatgcagatcttcgtgaagacacttactggcaagacgatcacccttgaggtggagtcctcggacaccatcgacaatgtcaaggccaagatccaggacaaggagggcattccaccggaccagcagcggctcatctttgctggcaagcagctggaggatggccgtacccttgcagattacaacatccagaaggagagcaccctccacttggtgctgcgtctcaggggtggcatgcagatcttcgtgaagactcttactggcaagacgatcacccttgaggtggagtcctcagacaccattgacaacgtcaaggccaagatccaggacaaggagggcattccaccggaccagcagcggctgatcttcgccggcaagcagctggaggatggtcgcaccttggctgactacaacatccagaaggagagcaccctccacttggtgctccgcctcaggggtggcatgcagatctttgtgaagacattgactggcaagaccatcaccttggaggtggagtcgtctgacacaatcgataatgtcaaagccaagatccaggacaaggagggcattccaccggaccagcagcgcctgatctttgctggcaagcagctggaggatggccgtaccctcgccgactacaacatccagaaggagagcaccctccacctggtgctccgcttgcgaggtggtatgcagatctttgtgaagaccttgactggcaagaccataaccctggaggttgagagctccgacaccatcgacaacgtgaaggcgaagatccaggacaaagagggcatccccccagaccagcagcgtctgatcttcgccggcaagcagctggaggatggccgcaccctagcagactacaacatccagaaggagagcactctccacctggtgctccgcctccgcggtggtcagtaagccatggttcatttaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattatgaataagctgtcagttaaaagtctgctaaatagggctgtttccggttatttctcttgatgttgcgtgaggatagatttcttcagctggtgttcatcagacatccagattcgtgaggacagatttgttcagctggtgtttgtgtttcattagacatccaagtgtcgttcctgggcagagacgccccaggttcctcggtagacgcaagaacttgtctagctggtattccatgattccatcagacactgtaggcccccctacagtaatttttacttcaaaaaaatgattccatcagacctcccgtttcttactagctaaaacttcagttcactgaatgctattagctagctacaggatctgaaaagctcggtcctttagtccggcttacacttcgttgcagcgctactgggttctgatcgctggttcttgctaaatccaaaatatcgcgtaggattgtatttctttctggtggctgaattggagggcaggctatggcttcatgttttctcgagaaaaattggctgcaaggtggtgaccagctgctgcctgctcatcagccgagtgcacgcagccgcaggtgatgcgcaattgcgcatgctttgcctacctcacagtcacagcttccatcccctctcggtaaaagaggagctgctgcatacagcgcgtggcccaagaaccgagaggatgcctgaaggagatcttcgctttttgatcggctcactctttctccacgacagagcccgttgatcgaccagtctcatcacaagaccggcccacgcacagaagagtgacgattgtgcgtcggtgacgtatacgccgcatggaaacgagaacagtgcagcgaattaattaattaagaagatgatgacctgaaagaggcgatgatgagccgctgaaagttgcgcagaagaggccggggccagccagctagcgagctagggggggctttgaggccatggcaggaaaggaaaggaaaggatcatgttcttttcttgctctttcttccacccactgcagacgccgacgccgatgcctcaacctcaacaaccgcaaccatggacggtgaggactgcaaaagagaccgcacgaatcgcattcctggtgggcacacacgcacacgtgctgcctggctaggactagctaggagtgggcgggtgaaagcgctgcgaaacttaaccgggatgcagct

SEQ ID NO: 46 - DNA, 6123 bp, Zea mays
  source 1..6123: mol_type = genomic DNA
cgtcctctaaatttagagaaccgtttagaggacactgctggagggagtaaaggacgtcctcgttctctaaatttagggtacagaactatttagagctacttgttggagctagccttaatatcgcagttagccttcaaaagaaatgcatttgcagccggcctgtcctgtccccgactaatctgctagcagactcgcattatcgatggatgacactaataaattcagccttcgacgtggatgcaacagctgcacaggattctattaaatcgtagccgtcgtgtcaaagtttgctttgccaacgtttttttttatttagaaaaccagctttgaccagccgccctctttacgtttggcacaatttagctgaatccggcggcatggcaaggtagactgcagcgcagcgtgacccggtcgtgcccctctctagagataaagagcattgcatgtctaagttataaaaaattaccacaattttttaagtgcagtttacgtatctctatacatatatttaaactttactatacgaataatatagtttataatactaaaataatatcagtgttttagagaattatataaatgaactgctagacatggtctaaataacaattgagtgttttgacaacaggactctacagttttatctttttagtgtgcatgtgtcctattttttttttgcaaatagcttcacctatataatacttcaccaatttattagtacatccatttagggtttagggttaatggtttctatagactaatttttagtacatctattttattctattttagcctctaaattaagaaaactaaaactttattttagtttttttaataatttagatataaatagaataaaataaagtgactaaaaattaactaaataccttttaaaaaaataaaaaactaaggaaacatttttcttgttccgagtagataatgacaggctgttcaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacacaaccagatctcccccaaatccacccgtcggcacctccgcttcaaggtacgccgctcatcctccccccccccccccccctctctctaccttctctagatcggcgttccggtccatggttagggcccggtagttctacttctgttcatgtttgtgttagatccgtgtttgtgttagatccgtgctgctagcgttcgtacacggatgcgacctgtacatcagacatgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgtttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgctttttttttcgcttggttgtgatgatgtggtctggttgggcggtcgttctagatcggagtagaatactgtttcaaactaactggtggatttattaattttggatctgtatgtgtgtgccatacatcttcatagttacgagtttaagatgatggatggaagtatcgatctaggataggtatacatgttgatgttggttttactgatgcatatacatgatggcatatgcagcatctattcattcatatgctctaaccttgagtacctatctattataataaacaagtatgttttataattattttgatcttgatatacttggatgatggcatatgcagcagctatatgtggatttttttagctctgccttcatacgctatttatttgcttggtactgtttcttttgtcgatgctcaccctgttgtttggtgatacttctgcagatgcagatctttgtgaaaaccctgaccggcaagactatcaccctcgaggtggagtcgtctgacaccattgacaacgttaaggccaagatccaggacaaggagggcatccccccagaccagcagcggctcatcttcgctggcaaacagcttgaggacgggcgcacgcttgctgactacaacatccagaaggagagcaccctccaccttgtgctccgcctcaggggaggcatgcagatctttgtgaaaaccctgaccggcaagactatcaccctcgaggtggagtcctctgacaccattgacaacgtcaaggccaagatccaggacaaggagggcatccctccagaccagcagcggctcatctttgctgggaagcagcttgaggacgggcgcacgcttgccgactacaacatccagaaggagagcaccctccacttggtgctgcgcctcaggggaggcatgcagatcttcgtgaagaccctgaccggcaaaactatcaccctcgaggtggagtcttcagacaccatcgacaacgtcaaggccaagatccaggacaaggagggcattcccccagaccagcagcggctcatctttgctggaaagcagcttgaggacgggcgcacgctggccgactacaacatccagaaggagagcaccctccacttggtgctgcgcctcaggggaggcatgcagatcttcgtgaagaccctgactggcaagactatcaccctcgaggtggagtcttcagacaccatcgacaacgtcaaggccaagatccaggacaaggagggcatcccaccggaccagcagcgtttgatcttcgctggcaagcagctggaggatggccgcacccttgcggattacaacatccagaaggagagcaccctccacctggtgctccgtctcaggggtggcatgcagatctttgtgaagacactcactggcaagacaatcacccttgaggtggagtcttcggataccattgacaatgtcaaggccaagatccaggacaaggagggcatcccaccggaccagcagcgcctcatcttcgccggcaagcagctggaggatggccgcaccctggcggactacaacatccagaaggagagcaccctccacctggtgctccgcctcaggggtggcatgcagatttttgtgaagacattgactggcaagaccatcaccttggaggtggagagctctgacaccattgacaatgtgaaggccaagatccaggacaaggagggcattcccccagaccagcagcgtctgatctttgctggcaagcagctggaggatggtcgcaccctcgcggactacaacatccagaaggagagtacccttcaccttgttctccgcctcaggggtggtatgcagatctttgtgaagaccctgactggaaaaaccataaccctggaggttgagagctcggacaccatcgacaatgtgaaggcgaagatccaggacaaggagggcatccccccggaccagcagcgtctgatcttcgccggcaaacagctggaggatggccgcaccttagcagactacaacatccaaaaggagagcaccctccaccttgtgctccgtctccgtggtggtcagtaagtcatgggtcgtttaagctgccgatgtgcctgcgtcgtctggtgccctctccatatggaggttgtcaaagtatctgctgttcgtgtcatgagtcgtgtcagtgttggtttaataatggaccggttgtgttgtgtgtgcgtactacccagaactatgacaaatcatgaataagtttgttgtttgaaattgaagcctgtgctcattatgttttatctttcagttgtctcctaatatttgcctgcaggttctggctatcaaccgtttcttttgttaacgtggttttgtcaagctcgggttttgacactggcctggcttacagcttaacatggttgattgttttgtttgttgctaagctattgggttctgattgcttgttcttgctgaatccaaaagatctcgtagtattgctcagattcatattatttatttgagtgataagtgatccaggttattactatgttagctaggttttttttacaaggataaattatctggatgctttctgtgatcataattcctgtgaaagctttatgtttcctggaggcagtggcatgcaatgcatgacagcaacttgatcacaccagctgaagtagatagcaaggttcttaaatctctcgccaaatcattggagaacacacatgcacattcttgccagtcttggttagagaaatttcatgacaaaatgccaaagctgtcttgactcttcacttttgtccatgagtcgtgactgagtttggtttaatggaccggttctcctagcttgttctactcaagtttgtttggctggtgttcatcagtttatagctgtctaccaaggataaatttatttggatggtgttcatcagacatccaggttcctagatgggcgcatagatttgtttgaccggtgttctatccggcctccagattgtgggcttgctactacaggatctgttctactcaagtttgtttggctggtgttcatcagtttgtagctgtctaccaaggataaatttatttggatggtgttcatcagacatccaggttcctagatgggcgcatagatttgtttgaccggtgttctatccggcctccagattgtgggcttgctactacaggatctggaaagctcagggtgtacagtttgttgctgtactgggttctgattggtagatctagctaaatccaaaaatatcatgtaggattgctgcaagatagaggggatgcagtggaagtggtctgaataaccttatgtgtcaaatactgctaactgctagaagcgaaattgctattgagagagaaaaaatcactgcttcgtatgcttccgcatgtaccttgctgatcactgtgggcaaccctcctaagcagtccctaaaatttctaggaagcctctttttagttataccgtttgtcagtttaaggaataaaagtgactaaattgctaaaggttaggaaagtttaggagctgggaactaaacacccctctaaatttgatggcttttgactcgtggttgtgacagaatgctaggaattttttttcaggtcatactgatcacatagacaactaatttcttttatctcaccataaaaaaaagaaactgcagaacggttcaaggtttgctgaaatctggagggcacgccgatatatattgcagaattccattggctgcttgctcatcggaatgcatgcagcgggctacagacacctgcaccggttattgcatcagaatgcattcgactctcctctctctatactgtcaggacactcgaaggaaatcgttgccttttgatcggctcactctttcacctcttctattagtactgtaagttcaggtgaagaccggagctcagagagagaagttagaccttctagtctacttgtagtttatttaaaaacacttaggccccgtttggattattgaaatttaattctattttaataatagtaattttagcatatatggattaagctaattcgattttgtatatatttatgtgttatatttttattataaaagaatgaaacgaatgatatgatgtaagttacagagtagaaacaaattctactattacatgaaatcatttctcattctcaacccatgattttgagatagacttatatctgaactgtagaaagtgatggaatatcaaattctagactaaataagttactttattgag

SEQ ID NO: 47 - DNA, 20 bp, Zea mays
  source 1..20: mol_type = genomic DNA
ccatggcttactgaccaccg

SEQ ID NO: 48 - DNA, 20 bp, Zea mays
  source 1..20: mol_type = genomic DNA
ccatgacttactgaccacca

SEQ ID NO: 49 - DNA, 572 bp, Zea mays
  source 1..572: mol_type = genomic DNA
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgtttt

SEQ ID NO: 50 - DNA, 3537 bp, synthetic construct
  misc_feature 1..3537: note = Cry1Ac codon optimised
  source 1..3537: mol_type = other DNA
atggacaacaaccctaacatcaacgagtgtattccctataattgcttgtccaatccggaagtcgaggtgctcggcggggagcggatcgaaaccggctacaccccaatcgatatctcgctttcgctgacacagttcctgctctccgagtttgtcccaggggcaggctttgtgttgggcctggtggatatcatttggggcatctttgggccatcacaatgggacgcgtttcttgtgcagatcgagcagctcatcaatcagcggatcgaagagttcgcaagaaaccaggcgattagcaggttggaaggtctctccaacttgtaccagatatatgccgagtcatttcgggagtgggaggccgatcccacaaacccagcactgagggaggagatgcggatacagttcaacgacatgaactcagcgctcaccacagcgatacctctgtttgcagttcaaaattatcaggtgccgctcctctcagtctacgtgcaggcagcgaatctgcacctcagcgtgcttagggatgtgtcagtcttcggccagcggtggggcttcgacgcagcgacaattaacagccgctataacgacctgacccggttgattggtaactacaccgattatgcagtccgctggtacaacacaggattggaaagagtgtggggacctgactccagagactgggtgagatacaaccagttcaggagagagctgactctcactgtcctggacatcgtggctctgtttcccaactacgacagcagacggtacccaatcaggaccgtgtcgcagctcactcgcgagatctacaccaatccggtgctggaaaattttgatggttcgtttcggggaagcgcgcaaggaatcgaacggagcatcagatcgcctcatctcatggacatcctgaactcaatcacgatctataccgacgcgcaccggggatactattattggtcgggacaccagattatggcatcgccagtcggtttttcggggccagaattcacctttccgctttatggtacaatggggaacgcggccccacaacaaaggatagtggcgcaactcggtcagggtgtgtatagaacattgagctcaacactgtacagaaggcccttcaacatagggatcaataaccagcaactgagcgttctggatgggaccgagtttgcgtacggaacaagctcaaatctcccctcagcggtgtatcgcaagtcgggtactgtggacagcctcgacgaaatccctccgcaaaacaataacgtgccgccgagacaagggtttagccaccggctgtcacacgttagcatgttccggagcggcttctcgaacagctcggttagcataatccgcgcgccaatgttttcctggatccaccgcagcgcggagtttaacaatattatcgcaagcgacagtataacccagatccccgcggtcaaggggaacttcctgtttaacggctcagtcatcagcggacccggttttactggcggtgacctcgtgaggctgaacagctccggtaataacatacagaatcgcggttatatagaggtgcccatccacttcccgagcacgtcgactcggtatagagtccgggttagatatgcatcagttacacccatacacctgaacgtgaattggggcaacagctcgatcttcagcaatacagtgccagccaccgcaactagcctcgacaacctgcagagctcggactttggctacttcgaatctgcgaacgcctttacctcgagcctggggaatatcgtcggtgttcggaatttttcgggaaccgcaggtgtgatcatagataggttcgagtttatcccggtgactgcaacactggaggcggaatacaatctggaacgggcacaaaaggcagtgaatgcgcttttcacgtcaactaaccagctgggtctcaagaccaacgtgacagattaccatattgaccaggtcagcaatctcgtcacttacttgtcagacgaattctgtctcgatgaaaagagagaactgagcgagaaggttaaacacgcgaaaagactctcggacgaaaggaaccttttgcaggactcgaatttcaaggacatcaacagacagccagagcggggttggggtggaagcactggcattactatccaaggtggagatgatgtgtttaaggaaaactacgtgacactgtcagggacatttgacgaatgctatccgacatacttgtaccagaaaatcgatgagtcgaagcttaaggcgtttacccggtatcaactgcggggctatatcgaggatagtcaagacctcgaaatctacctcatacggtataacgcgaagcatgagacggttaacgtgcccggtacggggagcctttggccactctcagcacagagccccatcgggaagtgtggtgagcccaatcggtgcgcgcctcatctggagtggaatccagacctggactgcagctgcagggatggggagaagtgtgcgcaccattcccaccacttcagcctggatatcgacgtcgggtgtaccgacctgaacgaggatctcggagtgtgggtcatctttaagataaaaacacaggacggtcatgcgcggttgggcaatctggagttccttgaggaaaaaccccttgttggcgaggcgctggccagagtgaagcgggcggaaaagaagtggcgcgacaagcgggagaagcttgaatgggagactaatatcgtgtataaggaagccaaagagtccgtggatgcgcttttcgtgaattcacaatatgatcagctccaagccgacaccaacatcgccatgattcatgccgcagataaacgggtgcactctatccgggaagcttatctccccgagttgtccgtgatccccggtgtcaacgcggcgatcttcgaagagttggaaggtaggatcttcaccgcctttagtctttatgacgcaagaaacgtgatcaagaacggggactttaacaacggtcttagctgctggaacgtcaaagggcacgttgacgtggaggaacagaacaaccaacggtcagttttggtggttccggagtgggaagcggaagtgagccaagaagtccgggtgtgccctggtagaggctacatcctcagagtgacagcgtacaaagaagggtacggtgaagggtgcgtgacaattcacgagatcgagaataacactgatgagctgaagtttagcaattgcgttgaagaggagatctatcccaacaataccgtgacatgcaatgactatacagtgaaccaggaagaatacggtggcgcttatacaagtagaaatcgcgggtacaacgaagcgcccagcgtgccagctgactatgcctcggtgtacgaagaaaaatcatatacagacggccgccgggaaaacccatgcgagtttaacagagggtatcgcgattatactccactgccagtgggctacgtcacgaaggagctcgaatatttcccagaaacagataaagtgtggatcgaaataggagagaccgagggtaccttcatcgtggactcagtcgaactccttctcatggaggaataa

SEQ ID NO: 51 - protein, 1178 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 52 - DNA, 3696 bp, synthetic construct
  misc_feature 1..3696: note = RCA-Cry1Ac codon optimised
  source 1..3696: mol_type = other DNA
atggcggccgccttctcctccaccgtcggagctccggcctccaccccgaccaggagcagcttcctcgggaagaagctcaacaagccgcaggtgtccgcggccgtgacctaccatggcaagagctccagcagcaacagcaggttcaaggcgatggctgccatggacaacaaccctaacatcaacgagtgtattccctataattgcttgtccaatccggaagtcgaggtgctcggcggggagcggatcgaaaccggctacaccccaatcgatatctcgctttcgctgacacagttcctgctctccgagtttgtcccaggggcaggctttgtgttgggcctggtggatatcatttggggcatctttgggccatcacaatgggacgcgtttcttgtgcagatcgagcagctcatcaatcagcggatcgaagagttcgcaagaaaccaggcgattagcaggttggaaggtctctccaacttgtaccagatatatgccgagtcatttcgggagtgggaggccgatcccacaaacccagcactgagggaggagatgcggatacagttcaacgacatgaactcagcgctcaccacagcgatacctctgtttgcagttcaaaattatcaggtgccgctcctctcagtctacgtgcaggcagcgaatctgcacctcagcgtgcttagggatgtgtcagtcttcggccagcggtggggcttcgacgcagcgacaattaacagccgctataacgacctgacccggttgattggtaactacaccgattatgcagtccgctggtacaacacaggattggaaagagtgtggggacctgactccagagactgggtgagatacaaccagttcaggagagagctgactctcactgtcctggacatcgtggctctgtttcccaactacgacagcagacggtacccaatcaggaccgtgtcgcagctcactcgcgagatctacaccaatccggtgctggaaaattttgatggttcgtttcggggaagcgcgcaaggaatcgaacggagcatcagatcgcctcatctcatggacatcctgaactcaatcacgatctataccgacgcgcaccggggatactattattggtcgggacaccagattatggcatcgccagtcggtttttcggggccagaattcacctttccgctttatggtacaatggggaacgcggccccacaacaaaggatagtggcgcaactcggtcagggtgtgtatagaacattgagctcaacactgtacagaaggcccttcaacatagggatcaataaccagcaactgagcgttctggatgggaccgagtttgcgtacggaacaagctcaaatctcccctcagcggtgtatcgcaagtcgggtactgtggacagcctcgacgaaatccctccgcaaaacaataacgtgccgccgagacaagggtttagccaccggctgtcacacgttagcatgttccggagcggcttctcgaacagctcggttagcataatccgcgcgccaatgttttcctggatccaccgcagcgcggagtttaacaatattatcgcaagcgacagtataacccagatccccgcggtcaaggggaacttcctgtttaacggctcagtcatcagcggacccggttttactggcggtgacctcgtgaggctgaacagctccggtaataacatacagaatcgcggttatatagaggtgcccatccacttcccgagcacgtcgactcggtatagagtccgggttagatatgcatcagttacacccatacacctgaacgtgaattggggcaacagctcgatcttcagcaatacagtgccagccaccgcaactagcctcgacaacctgcagagctcggactttggctacttcgaatctgcgaacgcctttacctcgagcctggggaatatcgtcggtgttcggaatttttcgggaaccgcaggtgtgatcatagataggttcgagtttatcccggtgactgcaacactggaggcggaatacaatctggaacgggcacaaaaggcagtgaatgcgcttttcacgtcaactaaccagctgggtctcaagaccaacgtgacagattaccatattgaccaggtcagcaatctcgtcacttacttgtcagacgaattctgtctcgatgaaaagagagaactgagcgagaaggttaaacacgcgaaaagactctcggacgaaaggaaccttttgcaggactcgaatttcaaggacatcaacagacagccagagcggggttggggtggaagcactggcattactatccaaggtggagatgatgtgtttaaggaaaactacgtgacactgtcagggacatttgacgaatgctatccgacatacttgtaccagaaaatcgatgagtcgaagcttaaggcgtttacccggtatcaactgcggggctatatcgaggatagtcaagacctcgaaatctacctcatacggtataacgcgaagcatgagacggttaacgtgcccggtacggggagcctttggccactctcagcacagagccccatcgggaagtgtggtgagcccaatcggtgcgcgcctcatctggagtggaatccagacctggactgcagctgcagggatggggagaagtgtgcgcaccattcccaccacttcagcctggatatcgacgtcgggtgtaccgacctgaacgaggatctcggagtgtgggtcatctttaagataaaaacacaggacggtcatgcgcggttgggcaatctggagttccttgaggaaaaaccccttgttggcgaggcgctggccagagtgaagcgggcggaaaagaagtggcgcgacaagcgggagaagcttgaatgggagactaatatcgtgtataaggaagccaaagagtccgtggatgcgcttttcgtgaattcacaatatgatcagctccaagccgacaccaacatcgccatgattcatgccgcagataaacgggtgcactctatccgggaagcttatctccccgagttgtccgtgatccccggtgtcaacgcggcgatcttcgaagagttggaaggtaggatcttcaccgcctttagtctttatgacgcaagaaacgtgatcaagaacggggactttaacaacggtcttagctgctggaacgtcaaagggcacgttgacgtggaggaacagaacaaccaacggtcagttttggtggttccggagtgggaagcggaagtgagccaagaagtccgggtgtgccctggtagaggctacatcctcagagtgacagcgtacaaagaagggtacggtgaagggtgcgtgacaattcacgagatcgagaataacactgatgagctgaagtttagcaattgcgttgaagaggagatctatcccaacaataccgtgacatgcaatgactatacagtgaaccaggaagaatacggtggcgcttatacaagtagaaatcgcgggtacaacgaagcgcccagcgtgccagctgactatgcctcggtgtacgaagaaaaatcatatacagacggccgccgggaaaacccatgcgagtttaacagagggtatcgcgattatactccactgccagtgggctacgtcacgaaggagctcgaatatttcccagaaacagataaagtgtggatcgaaataggagagaccgagggtaccttcatcgtggactcagtcgaactccttctcatggaggaataa

SEQ ID NO: 53 - DNA, 5041 bp, synthetic construct
  misc_feature 1..5041: note = ZmUbiChr5-Cry1Ac -ZmUbiChr5 donor repair fragment flanked by gRNA20 target sites
  source 1..5041: mol_type = other DNA
ccgtctccgtggtggtcagtaagtcatgggtcgttatgcagatctttgtgaagacactcactggcaagacaatcacccttgaggtggagtcttcggataccattgacaatgtcaaggccaagatccaggacaaggagggcatcccaccggaccagcagcgcctcatcttcgccggcaagcagctggaggatggccgcaccctggcggactacaacatccagaaggagagcaccctccacctggtgctccgcctcaggggtggcatgcagatttttgtgaagacattgactggcaagaccatcaccttggaggtggagagctctgacaccattgacaatgtgaaggccaagatccaggacaaggagggcattcccccagaccagcagcgtctgatctttgctggcaagcagctggaggatggtcgcaccctcgcggactacaacatccagaaggagagtacccttcaccttgttctccgcctcaggggtggtatgcagatctttgtgaagaccctgactggaaaaaccataaccctggaggttgagagctcggacaccatcgacaatgtgaaggcgaagatccaggacaaggagggcatccccccggaccagcagcgtctgatcttcgccggcaaacagctggaggatggccgcaccttagcagactacaacatccaaaaggagagcaccctccaccttgtgctccgtctccgtggtggtatggacaacaaccctaacatcaacgagtgtattccctataattgcttgtccaatccggaagtcgaggtgctcggcggggagcggatcgaaaccggctacaccccaatcgatatctcgctttcgctgacacagttcctgctctccgagtttgtcccaggggcaggctttgtgttgggcctggtggatatcatttggggcatctttgggccatcacaatgggacgcgtttcttgtgcagatcgagcagctcatcaatcagcggatcgaagagttcgcaagaaaccaggcgattagcaggttggaaggtctctccaacttgtaccagatatatgccgagtcatttcgggagtgggaggccgatcccacaaacccagcactgagggaggagatgcggatacagttcaacgacatgaactcagcgctcaccacagcgatacctctgtttgcagttcaaaattatcaggtgccgctcctctcagtctacgtgcaggcagcgaatctgcacctcagcgtgcttagggatgtgtcagtcttcggccagcggtggggcttcgacgcagcgacaattaacagccgctataacgacctgacccggttgattggtaactacaccgattatgcagtccgctggtacaacacaggattggaaagagtgtggggacctgactccagagactgggtgagatacaaccagttcaggagagagctgactctcactgtcctggacatcgtggctctgtttcccaactacgacagcagacggtacccaatcaggaccgtgtcgcagctcactcgcgagatctacaccaatccggtgctggaaaattttgatggttcgtttcggggaagcgcgcaaggaatcgaacggagcatcagatcgcctcatctcatggacatcctgaactcaatcacgatctataccgacgcgcaccggggatactattattggtcgggacaccagattatggcatcgccagtcggtttttcggggccagaattcacctttccgctttatggtacaatggggaacgcggccccacaacaaaggatagtggcgcaactcggtcagggtgtgtatagaacattgagctcaacactgtacagaaggcccttcaacatagggatcaataaccagcaactgagcgttctggatgggaccgagtttgcgtacggaacaagctcaaatctcccctcagcggtgtatcgcaagtcgggtactgtggacagcctcgacgaaatccctccgcaaaacaataacgtgccgccgagacaagggtttagccaccggctgtcacacgttagcatgttccggagcggcttctcgaacagctcggttagcataatccgcgcgccaatgttttcctggatccaccgcagcgcggagtttaacaatattatcgcaagcgacagtataacccagatccccgcggtcaaggggaacttcctgtttaacggctcagtcatcagcggacccggttttactggcggtgacctcgtgaggctgaacagctccggtaataacatacagaatcgcggttatatagaggtgcccatccacttcccgagcacgtcgactcggtatagagtccgggttagatatgcatcagttacacccatacacctgaacgtgaattggggcaacagctcgatcttcagcaatacagtgccagccaccgcaactagcctcgacaacctgcagagctcggactttggctacttcgaatctgcgaacgcctttacctcgagcctggggaatatcgtcggtgttcggaatttttcgggaaccgcaggtgtgatcatagataggttcgagtttatcccggtgactgcaacactggaggcggaatacaatctggaacgggcacaaaaggcagtgaatgcgcttttcacgtcaactaaccagctgggtctcaagaccaacgtgacagattaccatattgaccaggtcagcaatctcgtcacttacttgtcagacgaattctgtctcgatgaaaagagagaactgagcgagaaggttaaacacgcgaaaagactctcggacgaaaggaaccttttgcaggactcgaatttcaaggacatcaacagacagccagagcggggttggggtggaagcactggcattactatccaaggtggagatgatgtgtttaaggaaaactacgtgacactgtcagggacatttgacgaatgctatccgacatacttgtaccagaaaatcgatgagtcgaagcttaaggcgtttacccggtatcaactgcggggctatatcgaggatagtcaagacctcgaaatctacctcatacggtataacgcgaagcatgagacggttaacgtgcccggtacggggagcctttggccactctcagcacagagccccatcgggaagtgtggtgagcccaatcggtgcgcgcctcatctggagtggaatccagacctggactgcagctgcagggatggggagaagtgtgcgcaccattcccaccacttcagcctggatatcgacgtcgggtgtaccgacctgaacgaggatctcggagtgtgggtcatctttaagataaaaacacaggacggtcatgcgcggttgggcaatctggagttccttgaggaaaaaccccttgttggcgaggcgctggccagagtgaagcgggcggaaaagaagtggcgcgacaagcgggagaagcttgaatgggagactaatatcgtgtataaggaagccaaagagtccgtggatgcgcttttcgtgaattcacaatatgatcagctccaagccgacaccaacatcgccatgattcatgccgcagataaacgggtgcactctatccgggaagcttatctccccgagttgtccgtgatccccggtgtcaacgcggcgatcttcgaagagttggaaggtaggatcttcaccgcctttagtctttatgacgcaagaaacgtgatcaagaacggggactttaacaacggtcttagctgctggaacgtcaaagggcacgttgacgtggaggaacagaacaaccaacggtcagttttggtggttccggagtgggaagcggaagtgagccaagaagtccgggtgtgccctggtagaggctacatcctcagagtgacagcgtacaaagaagggtacggtgaagggtgcgtgacaattcacgagatcgagaataacactgatgagctgaagtttagcaattgcgttgaagaggagatctatcccaacaataccgtgacatgcaatgactatacagtgaaccaggaagaatacggtggcgcttatacaagtagaaatcgcgggtacaacgaagcgcccagcgtgccagctgactatgcctcggtgtacgaagaaaaatcatatacagacggccgccgggaaaacccatgcgagtttaacagagggtatcgcgattatactccactgccagtgggctacgtcacgaaggagctcgaatatttcccagaaacagataaagtgtggatcgaaataggagagaccgagggtaccttcatcgtggactcagtcgaactccttctcatggaggaataagtcatgggtcgtttaagctgccgatgtgcctgcgtcgtctggtgccctctccatatggaggttgtcaaagtatctgctgttcgtgtcatgagtcgtgtcagtgttggtttaataatggaccggttgtgttgtgtgtgcgtactacccagaactatgacaaatcatgaataagtttgttgtttgaaattgaagcctgtgctcattatgttttatctttcagttgtctcctaatatttgcctgcaggttctggctatcaaccgtttcttttgttaacgtggttttgtcaagctcgggttttgacactggcctggcttacagcttaacatggttgattgttttgtttgttgctaagctattgggttctgattgcttgttcttgctgaatccaaaagatctcgtagtattgctcagattcatattatttatttgagtgataagtgatccaggttattactatgttagctaggttttttttacaaggataaattatctggatgctttctgtgatcataattcctgtgaaagctttatgtttcctggaggcagtggcatgcaatgcatgacagcaacttgatcacaccagctgaagtagatagcaaggttcttaaatctctcgccaaatcattggagaacacacatgcacattcttgccagtcttggttagagaaatttcatgacaaaatgccaaagctgtcttgactcttcacttttgtccatgagtcgtgactgagtttggtttaatggaccggccgtctccgtggtggtcagtaagtcatgggtcgtt

SEQ ID NO: 54 - DNA, 5200 bp, synthetic construct
  misc_feature 1..5200: note = ZmUbiChr5-RCA-Cry1Ac -ZmUbiChr5 donor repair fragment flanked by gRNA20 target sites
  source 1..5200: mol_type = other DNA
ccgtctccgtggtggtcagtaagtcatgggtcgttatgcagatctttgtgaagacactcactggcaagacaatcacccttgaggtggagtcttcggataccattgacaatgtcaaggccaagatccaggacaaggagggcatcccaccggaccagcagcgcctcatcttcgccggcaagcagctggaggatggccgcaccctggcggactacaacatccagaaggagagcaccctccacctggtgctccgcctcaggggtggcatgcagatttttgtgaagacattgactggcaagaccatcaccttggaggtggagagctctgacaccattgacaatgtgaaggccaagatccaggacaaggagggcattcccccagaccagcagcgtctgatctttgctggcaagcagctggaggatggtcgcaccctcgcggactacaacatccagaaggagagtacccttcaccttgttctccgcctcaggggtggtatgcagatctttgtgaagaccctgactggaaaaaccataaccctggaggttgagagctcggacaccatcgacaatgtgaaggcgaagatccaggacaaggagggcatccccccggaccagcagcgtctgatcttcgccggcaaacagctggaggatggccgcaccttagcagactacaacatccaaaaggagagcaccctccaccttgtgctccgtctccgtggtggtatggcggccgccttctcctccaccgtcggagctccggcctccaccccgaccaggagcagcttcctcgggaagaagctcaacaagccgcaggtgtccgcggccgtgacctaccatggcaagagctccagcagcaacagcaggttcaaggcgatggctgccatggacaacaaccctaacatcaacgagtgtattccctataattgcttgtccaatccggaagtcgaggtgctcggcggggagcggatcgaaaccggctacaccccaatcgatatctcgctttcgctgacacagttcctgctctccgagtttgtcccaggggcaggctttgtgttgggcctggtggatatcatttggggcatctttgggccatcacaatgggacgcgtttcttgtgcagatcgagcagctcatcaatcagcggatcgaagagttcgcaagaaaccaggcgattagcaggttggaaggtctctccaacttgtaccagatatatgccgagtcatttcgggagtgggaggccgatcccacaaacccagcactgagggaggagatgcggatacagttcaacgacatgaactcagcgctcaccacagcgatacctctgtttgcagttcaaaattatcaggtgccgctcctctcagtctacgtgcaggcagcgaatctgcacctcagcgtgcttagggatgtgtcagtcttcggccagcggtggggcttcgacgcagcgacaattaacagccgctataacgacctgacccggttgattggtaactacaccgattatgcagtccgctggtacaacacaggattggaaagagtgtggggacctgactccagagactgggtgagatacaaccagttcaggagagagctgactctcactgtcctggacatcgtggctctgtttcccaactacgacagcagacggtacccaatcaggaccgtgtcgcagctcactcgcgagatctacaccaatccggtgctggaaaattttgatggttcgtttcggggaagcgcgcaaggaatcgaacggagcatcagatcgcctcatctcatggacatcctgaactcaatcacgatctataccgacgcgcaccggggatactattattggtcgggacaccagattatggcatcgccagtcggtttttcggggccagaattcacctttccgctttatggtacaatggggaacgcggccccacaacaaaggatagtggcgcaactcggtcagggtgtgtatagaacattgagctcaacactgtacagaaggcccttcaacatagggatcaataaccagcaactgagcgttctggatgggaccgagtttgcgtacggaacaagctcaaatctcccctcagcggtgtatcgcaagtcgggtactgtggacagcctcgacgaaatccctccgcaaaacaataacgtgccgccgagacaagggtttagccaccggctgtcacacgttagcatgttccggagcggcttctcgaacagctcggttagcataatccgcgcgccaatgttttcctggatccaccgcagcgcggagtttaacaatattatcgcaagcgacagtataacccagatccccgcggtcaaggggaacttcctgtttaacggctcagtcatcagcggacccggttttactggcggtgacctcgtgaggctgaacagctccggtaataacatacagaatcgcggttatatagaggtgcccatccacttcccgagcacgtcgactcggtatagagtccgggttagatatgcatcagttacacccatacacctgaacgtgaattggggcaacagctcgatcttcagcaatacagtgccagccaccgcaactagcctcgacaacctgcagagctcggactttggctacttcgaatctgcgaacgcctttacctcgagcctggggaatatcgtcggtgttcggaatttttcgggaaccgcaggtgtgatcatagataggttcgagtttatcccggtgactgcaacactggaggcggaatacaatctggaacgggcacaaaaggcagtgaatgcgcttttcacgtcaactaaccagctgggtctcaagaccaacgtgacagattaccatattgaccaggtcagcaatctcgtcacttacttgtcagacgaattctgtctcgatgaaaagagagaactgagcgagaaggttaaacacgcgaaaagactctcggacgaaaggaaccttttgcaggactcgaatttcaaggacatcaacagacagccagagcggggttggggtggaagcactggcattactatccaaggtggagatgatgtgtttaaggaaaactacgtgacactgtcagggacatttgacgaatgctatccgacatacttgtaccagaaaatcgatgagtcgaagcttaaggcgtttacccggtatcaactgcggggctatatcgaggatagtcaagacctcgaaatctacctcatacggtataacgcgaagcatgagacggttaacgtgcccggtacggggagcctttggccactctcagcacagagccccatcgggaagtgtggtgagcccaatcggtgcgcgcctcatctggagtggaatccagacctggactgcagctgcagggatggggagaagtgtgcgcaccattcccaccacttcagcctggatatcgacgtcgggtgtaccgacctgaacgaggatctcggagtgtgggtcatctttaagataaaaacacaggacggtcatgcgcggttgggcaatctggagttccttgaggaaaaaccccttgttggcgaggcgctggccagagtgaagcgggcggaaaagaagtggcgcgacaagcgggagaagcttgaatgggagactaatatcgtgtataaggaagccaaagagtccgtggatgcgcttttcgtgaattcacaatatgatcagctccaagccgacaccaacatcgccatgattcatgccgcagataaacgggtgcactctatccgggaagcttatctccccgagttgtccgtgatccccggtgtcaacgcggcgatcttcgaagagttggaaggtaggatcttcaccgcctttagtctttatgacgcaagaaacgtgatcaagaacggggactttaacaacggtcttagctgctggaacgtcaaagggcacgttgacgtggaggaacagaacaaccaacggtcagttttggtggttccggagtgggaagcggaagtgagccaagaagtccgggtgtgccctggtagaggctacatcctcagagtgacagcgtacaaagaagggtacggtgaagggtgcgtgacaattcacgagatcgagaataacactgatgagctgaagtttagcaattgcgttgaagaggagatctatcccaacaataccgtgacatgcaatgactatacagtgaaccaggaagaatacggtggcgcttatacaagtagaaatcgcgggtacaacgaagcgcccagcgtgccagctgactatgcctcggtgtacgaagaaaaatcatatacagacggccgccgggaaaacccatgcgagtttaacagagggtatcgcgattatactccactgccagtgggctacgtcacgaaggagctcgaatatttcccagaaacagataaagtgtggatcgaaataggagagaccgagggtaccttcatcgtggactcagtcgaactccttctcatggaggaataagtcatgggtcgtttaagctgccgatgtgcctgcgtcgtctggtgccctctccatatggaggttgtcaaagtatctgctgttcgtgtcatgagtcgtgtcagtgttggtttaataatggaccggttgtgttgtgtgtgcgtactacccagaactatgacaaatcatgaataagtttgttgtttgaaattgaagcctgtgctcattatgttttatctttcagttgtctcctaatatttgcctgcaggttctggctatcaaccgtttcttttgttaacgtggttttgtcaagctcgggttttgacactggcctggcttacagcttaacatggttgattgttttgtttgttgctaagctattgggttctgattgcttgttcttgctgaatccaaaagatctcgtagtattgctcagattcatattatttatttgagtgataagtgatccaggttattactatgttagctaggttttttttacaaggataaattatctggatgctttctgtgatcataattcctgtgaaagctttatgtttcctggaggcagtggcatgcaatgcatgacagcaacttgatcacaccagctgaagtagatagcaaggttcttaaatctctcgccaaatcattggagaacacacatgcacattcttgccagtcttggttagagaaatttcatgacaaaatgccaaagctgtcttgactcttcacttttgtccatgagtcgtgactgagtttggtttaatggaccggccgtctccgtggtggtcagtaagtcatgggtcgtt

SEQ ID NO: 55 - DNA, 2191 bp, Raphanus sativus
  source 1..2191: mol_type = genomic DNA
atgttggctagggtttgtggattcaagtgttcttcttctcctgctgagtctgcggctagattgttctgtacgagatcgattcgtgatactctggccaaggcaagcggagagagttgcgaagcaggttttggaggagagagtttgaagctgcaaagtgggtttcatgaaatcaaaggtttagaggatgcgattgatttgttcagtgacatgcttcgatctcgtcctttaccttctgtggttgatttctgtaaattgatgggtgtggtggtgagaatggaacgcccggatcttgtgatttctctctatcagaagatggaaaggaaacagattcgatgtgatatatacagcttcaatattctgataaaatgtttctgcagctgctctaagctcccctttgctttgtctacatttggtaagatcaccaagcttggactccaccctgatgttgttaccttcaccaccctgctccatggattatgtgtggaagatagggtttctgaagccttggatttttttcatcaaatgtttgaaacgacatgtaggcccaatgtcgtaaccttcaccactttgatgaacggtctttgccgcgagggtagaattgtcgaagccgtagctctgcttgatcggatgatggaagatggtctccagcctacccagattacttatggaacaatcgtagatgggatgtgtaagaagggagatactgtgtctgcactgaatctgctgaggaagatggaggaggtgagccacatcatacccaatgttgtaatctatagtgcaatcattgatagcctttgtaaagacggacgtcatagcgatgcacaaaatcttttcactgaaatgcaagagaaaggaatctttcccgatttatttacctacaacagtatgatagttggtttttgtagctctggtagatggagcgacgcggagcagttgttgcaagaaatgttagaaaggaagatcagccctgatgttgtaacttataatgctttgatcaatgcatttgtcaaggaaggcaagttctttgaggctgaagaattatacgatgagatgcttccaaggggtataatccctaatacaatcacatatagttcaatgatcgatggattttgcaaacagaatcgtcttgatgctgctgagcacatgttttatttgatggctaccaagggctgctctcccaacctaatcactttcaatactctcatagacggatattgtggggctaagaggatagatgatggaatggaacttctccatgagatgactgaaacaggattagttgctgacacaactacttacaacactcttattcacgggttctatctggtgggcgatcttaatgctgctctagaccttttacaagagatgatctctagtggtttgtgccctgatatcgttacttgtgacactttgctggatggtctctgcgataatgggaaactaaaagatgcattggaaatgtttaaggttatgcagaagagtaagaaggatcttgatgctagtcaccccttcaatggtgtggaacctgatgttcaaacttacaatatattgatcagcggcttgatcaatgaagggaagtttttagaggccgaggaattatacgaggagatgccccacaggggtatagtcccagatactatcacctatagctcaatgatcgatggattatgcaagcagagccgcctagatgaggctacacaaatgtttgattcgatgggtagcaagagcttctctccaaacgtagtgacctttactacactcattaatggctactgtaaggcaggaagggttgatgatgggctggagcttttctgcgagatgggtcgaagagggatagttgctaacgcaattacttacatcactttgatttgtggttttcgtaaagtgggtaatattaatggggctctagacattttccaggagatgatttcaagtggtgtgtatcctgataccattaccatccgcaatatgctgactggtttatggagtaaagaggaactaaaaagggcagtggcaatgcttgagaaactgcagatgagtatggtatgtaagtttctgttcagtctatgtattttttatataaacaagaatgtatacattcttttgtgtgtagcttcagattgatgatacacgttctggaattaaccattggtttggttttgcattgtaggatctatcatttgggggatga

SEQ ID NO: 56 - protein, 687 aa, Raphanus sativus
MLARVCGFKCSSSPAESAARLFCTRSIRDTLAKASGESCEAGFGGESLKLQSGFHEIKGLEDAIDLFSDMLRSRPLPSVVDFCKLMGVVVRMERPDLVISLYQKMERKQIRCDIYSFNILIKCFCSCSKLPFALSTFGKITKLGLHPDVVTFTTLLHGLCVEDRVSEALDFFHQMFETTCRPNVVTFTTLMNGLCREGRIVEAVALLDRMMEDGLQPTQITYGTIVDGMCKKGDTVSALNLLRKMEEVSHIIPNVVIYSAIIDSLCKDGRHSDAQNLFTEMQEKGIFPDLFTYNSMIVGFCSSGRWSDAEQLLQEMLERKISPDVVTYNALINAFVKEGKFFEAEELYDEMLPRGIIPNTITYSSMIDGFCKQNRLDAAEHMFYLMATKGCSPNLITFNTLIDGYCGAKRIDDGMELLHEMTETGLVADTTTYNTLIHGFYLVGDLNAALDLLQEMISSGLCPDIVTCDTLLDGLCDNGKLKDALEMFKVMQKSKKDLDASHPFNGVEPDVQTYNILISGLINEGKFLEAEELYEEMPHRGIVPDTITYSSMIDGLCKQSRLDEATQMFDSMGSKSFSPNVVTFTTLINGYCKAGRVDDGLELFCEMGRRGIVANAITYITLICGFRKVGNINGALDIFQEMISSGVYPDTITIRNMLTGLWSKEELKRAVAMLEKLQMSMDLSFGG

SEQ ID NO: 57 - DNA, 1316 bp, Brassica napus
  source 1..1316: mol_type = genomic DNA
aagattatctttccattaaaaggttgcgtgtagctaacgctcaatacgcgtcattttaacacgtgtcaaacttttattcgctattacttcacagccgtagctttctcgtgaccgagtcttcctcatcctctttgtataaatcaaaccctaaacacctcttctccttcacaaatcgataaaccaatcattctcaattctatagctctctctctctctcgcgaatttacgaatcaaaggttagatccgtgtttcttcttcttccgatcgcaattgctcatcagttatttgaatttgattcaatttgttgagattagtcgatagatctcttgttgatattgattattgcgattaaggttatattatcgtttcttcttcttcgattatctgttttgtacctatcgtattgatcctctgttcttcgaatttgaatcggtttgcgtatcgttcgtgtttgtatattgaagcgatctctttgttgttttcgcaattgatctttggctttttgtgaataacagatgcagatcttcgttaagactctcaccggaaagaccatcacccttgaggtggaaagctcggacaccatcgacaacgttaaagccaagattcaggacaaggagggaatccccccggatcagcaaaggcttatcttcgccggaaagcaactcgaggatggacgtactctcgctgattacaacatccagaaggagtcgacccttcatctcgtcctcaggctccgtggtggtatgcagatcttcgttaagactcttactggaaagacgatcacactcgaggtggagagctccgacaccactcttaccggaaagaccatcacacttgaggtggagagctcggacaccattgataacgtcaaggccaaaatccaagacaaggaagggatccctccagaccagcagcgtctcatcttcgctggcaagcagcttgaggatggtcgcacccttgccgactacaacattcagaaggagtcaacccttcacttggtgttgcgtctccgtggtggcttttaagtctttgtcctctgtcgtatcatgaaacaacaatgtctgcgtttttacttccgttttctttaaaacttatcatggttggtttgtgtgtttgaggcgttataaagcctctattaaataattgatgacctctgtttcctatctcttttaagaccattcgaatgtttccatgtcgactctattcttaaatgtttggttacgttgtagatgcggtgaaacttgctgttttgcttagctgttacttctgatagcataggggtgtttgtagcttgaagttgtcttcagaggtaggtg

SEQ ID NO: 58 - DNA, 7639 bp, Brassica napus
  source 1..7639: mol_type = genomic DNA
attcacaaatcgataaacaaatttctcaactcttatctctctcgcgaactacggttcaaggtatgattctcgtgtttttttcttcttcgatttagctgtttcattcaatcattatcagtcctctgtttttagagtttgattcggttatagctgtcatataaatataattgagctcagccgatagatctctttttgattttgataattattacgatcaacgttagattctggtttcttcttcttcgatatattagttgtttcgttctatcgctattgattctattgcagccatataattgagattagccgatagatctcgtcttgattttggttattagagttggttgtgttcgattcttcctgttctgtgaatgaaagcgatttctttgttgtttttgtgaataacagatgcagatcttcgtgaagactctcaccggaaagaccatcacccttgaggtggaaagctcggacaccatcgacaacgtcaaggccaagatccaggacaaggagggaatcccaccggatcagcaaaggcttatcttcgccgggaagcaactcgaggatggacgtactctcgctgattacaacatccagaaggagtcgacccttcatctcgtcctcaggctccgcggtggtatgcagatcttcgttaagactctcaccggaaagactttaaaattttataagtttctaaaaaattgtatacatttttgaaaattttagtactaaaattgtataatttaaaaatatataattaaattatatttcaaaatttatagtgctatatttgaatatatttattttaatgatgatttatgagttattctcatattctaaaaattttccaaaaatataaattgacattaaatgtaatatatgagttattaccatattttaaaaagtttaccaaaaatataaattaacattaaatgcaattgtccatgtcatattaagctataagacatgtcatcaatttcagtagtcatgtcatatttatttttagaaacgattttttctttttctttctgtcaaatagggcatagcaagtttataacaattctttaactttcatttatttgtaaatgccttgagtgatcaataaatttaaaatttcacccaaaaataaaataaaataaataggtaatatatagttgtatatataaaagtttaacatatgttaacaattttactttttgtataaaaaatattacataatttacgaattttaatctttttatacagtaaatgatattttttatttatttaaatgtaagtaaattttaacaaatatgttaatttaccaatttaatatattatcatatttaattcatatattacttctattttaatataatatagacttattataaaatttataagagatagcatagaattttattttatagttatataatagaattttatttaaaattaattgataataatattatattactaattagggaattaatcattgcgttaattttaaaaaatatatttaaaattgtagtaagattttcttagttttttatcaacagattttgttataattaaaagtaaattttaaatttacagttaaaattactttattattaatatctttataattattaacaattttttagagatgctatatatttaatagattaatgtaactaatcaaatagatgtaagtaatgaaattgactaacttttatggtagattaaaattgtacttttcttttaatagagaaaagtagtttttaatgtaatgtaaccgtatctgtttttcttggcttaattttaggaatatattttgtcctcgttaagattgttagcatcaaagtttaaaaaacattaacggtttttattttatatgaaaatttggtaactccatataacaccgcttttcacattccatttcatatggaaatttggtgattctaaattttcaagaatttactctatataatatattagaaatattttatatgaagtaataattcacattccataacgcaaaccgtttgtcacattccattttatatgaaaataagataattctaaattttcacacatctactctagaatagattaagatattttgtatgaagtagtgattcatggttttctaatatgcattagtgatgaatgttatgaatttattcatcacgaagagaagtcatggagaagaagaaacacatgtttatagattagattaaattctaatgagaacctttacaactttcgtagatacatacatgagacaatgagacaatgagacaatgagacaatgagacaaagaagaaactgttagaaaatagtatatttgtgattatccattaaaggataatgttgatttttataattttgtcattttgtgttaagtcggtaagaaatgaccgacattctcccctatatatagtgacttcatgtcacttgtaataattattagaaaatgtgaagaaagtcactttgctctcgttctattttcctctccaacaaaagaatacaataatataatatatatataaacatttctctacatataaatctcatcgcagaataatttacaacacgttatcagcacgaaactctggtcaattgaagtttatcaatccgaaagttcttaaaccagtcgaggtaatgtttaaatttatgtatttcaatatatttaatttatttaaattttattattatttaggagtgagaggctaggtcttctccgtctatttatcgggatgataggcgttgcctttcccgactgatcgttatggtaggctatgccttcacgatcagagatcacgtggtaggtgttgcctccgtgaataaaaaaagggtcaaaaatggtagattaagtctcctcggaccttgaaataagtaaaagtcaatggttggatttttaagttcttcacgaaaagaggaaaagaagtgattattaaatcagtgattacggctttaccaaatcatgtgatgtcttgctatcgtttacccaaggctactgcaaaaaaactgactagtgctgtcgcgcaattttggtggagtcctggcggcaacacaagaggaatgcattggaaatcatgggataaagtatgcatcagtaaggatgatggaggtttagggtttaaagacatcactgatttcaatacagcgatgcttggtaaacagttatggcgactgatagagaaaccaaatactttattttctcgagtttttaaaggtcggtactacaggaatgcatcacccctggatccgattcgttcatattctccgtcatatggatggcgaagtattgtatctgctagatctctggtaagcaaaggactaatcaaaagggtgggaacaggatcatctatatcagtatggaatgatccttggctcccaaccactcgcccgagaccagccaataaaaaccaacacaatctttacccagacctcacagtggattctcttatagatcctcatttgcgaagatggaattcgccggcactacgggctttggtggacccacaagatgtgaaattgatagaaagtataccattgagtagaactcggatggctgaccatgaaggatggcatttcacatataatggaaaatatacggttaaatctggatataaggttgaaaggatatacccagataagaaaaaacccccattagtgtttgggcccacagtggatattttgaaggcatattgttggaaaataaggtgtgccccgaaacttaaacattttctttggcaattggtgacaggatgcatagccgttaagaaaaatctgcatgggcgagggatacccgaagatatatgttgtgcaagatgtggagcagatgaggaatcgattaaccatgtcttttttgaatgtccccctgcacttcaggtatgggcgctttcgaagatcccaacaaatccagctctgttccccacaagctctctatttgtgaatatggatcatctcttttggagagttgtcccacaaatggaggattatcagtttgcctggatactttggtatatctggaaatgaagaaataataaaatatttagtaatattgatgtggatcttatggatacctttaaactggcggaaacggaatcaaaactatgggctgaggcacaaatattgaatgattaaaagaggacgacacatatagaggttaatattcttccgcaattccaggaagatggtgttttactgatggatcctggaaagaaaatgaaagtttctcaggtcaagggtggtacagcacattagaaggatttgcgggactaatgggaacaaggaatgtccgggcttcactcactcctctccatgcagagatggaagctttactatgggctatggaatgcatgaaaaacttaagacaatttcatgttacatttgcgacggattgttctcaattggtgaagatggtttcggaaccagaagaatggccagcgtttgcaagttatttggaagatatacacatcctgaaagagagttttactagtgcagagattatctatgtaccaagaacgctgaacaagaaggcggatggcttagccagaagcgctaggaaggaaacgtcttttgtagttcacatggatcaatatctccctgtgtggttcacagagtcaatatgagtctgtaaagttgatgacaaaaaaaaataaaaaaaataagtaaaagtcaaaatggtacaccatgtctcctcggattttgaaaaatgatcaatatggtagtctaagactcctcggatcatgtggtaggctaagcctccaattttaattattgctctttaaatttctgcaatttaatttatgcatttattttatgcttttaatttatgcatttaaatttccatctttatatattattattctatggatacggttatcatgtcaaaatttgcaaatctcaaaataaatgtccttgatattacgagaaataattataagacttgggtagtgggtgcaaagatgcacctgagagaaaatgagctttaagaaatcatcgataagttgaaacgatatcggatgagaaaaagaaaaaccatgatattttaccccacttcaatgatggtttataagatgaggtgtatcatgagaaaagatttagaagatctttgatagttaaatccttgaaagaaaggatcggagttgatgtaaatcctccgagtaaactgaaaaaaaaatgaaaacatgatactaaaccatcaagtcggtcatactagaaaaggacgagggtgtagatgcggttaaaaccgctatcatggtcgtggaagaggacgagaaagaagattccgtccctatgagaattatgagaacttccacgaaaatgaaagtggtagggtgataaaaggcaaacagaaaaagtgtgctatagatatggccagaaagaaaattgggtacgtagttatcgtacgccaatatttagccgatctatatcgagaatcaaaaagaaaaaagagaaaggaagtatattaaacttcgtctcttatgagcccgaaccatatttcatagcttgaatactcatcttgatgattcagattttctggttggtccagaaaatgaaaataaaatatcgatgtgatatgaaaattatcttcctgaataggattttgagattcaggatggagatagatatacctggcagatattgggtcatcgtacatgactactaaaggtaacaaatatttctcctctcccaaaataagtaagtataatatctattaatataaaaattattattggctctagaggagcttatgatgaaaagacatgcataaaaaaaaagatggcaaaagtataaatgagtggtaaacctgaagtttactgatatatagccatctcaacattattgtgaaatgctgaaaaactagagaataaaataaagaaaataaaggaaaccatcatgggtgatgaatcatcaaacaagttcattatatgtgatttctttaagcaaaccccaaattaagatctcactctaaaggatttgaaacataattcatcccaaatgggaaaactgaatatgttattagttctagagtgggattcagtacgagattttagacatgaaatgtcatcatctcgagggggagaattaaagaatcccagtgagtggaacattaagaaattatgtccgcacttgaaaaataacttgataaagtaaattcaagtaagatgatccccatgatcgggtgtaatgcagttgtgcatgtaataaagacatatacaatccagaaggataaagtatatggataattagaaatatgctcgcaagtttgctagaagcatatgataagctgatggaatgagatatatgaaatggattacaatgaaacgtagaataatccatttacaaaatgtctgcctgacattttaatgaaataaagaaatctcatattccagctgaaaatgctccaataagaaaatagtgtcctaaaaggacgatatatgagtctatggcacgctaaagacgtggtagaccattttggttccaaagagtcctcgaaaaggagcaaaaatataaatagaggatgaatatcatgatgagaccattgatatggctaatataatagtcaggtacctgggtaaatcattgatgatgataaagagatctcgattaaccatatcagtacggataaaaagatggaaccaaaaataaaatagattgtcgacaataaaatgaaaaaagcgctatataaataaggattatgaatctacctctatgtatgagggttgagtgaattgattggccatcaattcgatataaagctcgttagaaaaacgagagatttttggaccaaaagtccaaggtatatatttctccagagaatgaaattaaagatgaccttgaggtatgaaaaatggcttgttccaaggtcactggagaaaatttaaactagatgcaatataatgatatgtctggcaggacaagtcgtatcttgatatttagaagtccctggagagttaaagatgctcttgagaatgtataaaactctgaaagagttagaacaagccgtatataaggtatcttgaaccggtaactggtgatatgtttatggcacggttcaccgattgccatttaatgaggatgaatttccagcgttagggggatgaattagaaaaattcctaaagagattacatggtgtacatagtcgttgttacaccttgatcccatacgaatcaaagagagcttgaaattcaagaaattgtgcatttgcataatttggaaaaccagttaccaaatgtgttcacagatacgacatgatatacccgctgaaaatgttccatcaagggttgatgttcctaaagaacaaactgatggtaacaaaatgaatgagcctagggttcagttaaagagggggagactagcgggttctagagataaaaaaatccccgaaagaaaaagaaattggatgagcaaaatagtaaggttccaaaagaacctgatattgaaagatgtctgaaagaagaaataagtggaaaggttccagaagaacctgatcttgaaagatgtctgaaagaaggcataagtggaaagattccagaagaacctgatactgaaaatgtctgaaagaaggcatagatgaaaagcttcaagacgaaccaaataaagatgattcagatgacgagaagagaacagaaaagtatgagatttccatcaactatcctctaa

SEQ ID NO: 59 - DNA, 335 bp, Brassica napus
  source 1..335: mol_type = genomic DNA
gttgtataaaatgctcacatctctctaaaggcctattctccttcacaaatcgaaaaacaatcttctcaattctctaatttacgatcaagatgcagatcttcgttaagactctcaccgggaagacgatcaccctcgaggtggaaagctccgacaccatcgacaacgtcaaagccaagatccaggataaggaaggcattcccccggatcagcagaggttgatcttcgccggaaagcagcttgaagacggtagaactctcgctgactacaacatccagaaggagtctactctccacttggtgttgcgtcttcgtggaggtatgcagatcttcgtcaag

SEQ ID NO: 60 - DNA, 4640 bp, Brassica napus
  source 1..4640: mol_type = genomic DNA
tctgcaactaaaagacattactaataagataacatttgatctgaaaaacaggaacttgttgttattgacctacttcttggtaaaataggtaaaatcatgcacgcttcaatttgagctttgaatagtgtttgaaaaggcttttctatcggatgatctaatcgacaaacattagttgttttatataaaaaagttattgataatctttagttaagaggtataagatgaaaagattttcaatcaaatgtaaaatatttggtcgaacaaaaaggaaaaaagcttgttcatgtcaagggaactcgctcccagagatattgggatactttttgtatccacaagctaaaaaagctggaactttgcgtgtgctacaagaaacaaataggtgctgaacctttgaggttattttttcagagtgaagaagaaagaatcttaacgaggagtgatagtattggtcaaaagattatctgtagaacagtcgctgttttgaatgataatccatggactaatgtgttccgattcaggtatgttcatgaagaacaatccaggtgatgtaggcacatgaaatgtaacggagctataggtgatcatctggaaattgaatctaatgactcctcacagtaataatctgccaaattcacataagctgttgtagacaagaacttaggacacatgtgtaaccagattacaaactgagctatctatttaatagaaactgctacacagtgtagcctacacttcttgccactaacaatctcaagaaccaatacgtcttgtccaatctagcttcctctttcagacgcatctttcgtacttctgtttggaaaaacaacataaagccccctcctgtcagaagtactatcaacaaataaaatcattcgacaaggctttcattgactgaataaacccgatatcttaaatacgagacaggcccaaacaaggtccgttgatgtgtaaattaacttaaatatttagacccatttgccaatttgataccaaaatcaagcaaatatccttaaccactaaaaaacaaaattattttcaggaaaagttcttaatttgaaactcaaaactaaaaaccccatttgccaacttgataccacaatcatggaaatatccaaaaccactaaaaaatcataaacattatgttattttcaggaagattccattatttttaaattaattaaaaactccacttgcattaaaaaggaaaaataaagaaaagaaagataattggcacggttcaattattggcatttttagctccaccgttgatttaaaataaaagaaagataataatagcatcgatcttaacgatccgttaaatgacaacgtcggcttctgttacggcccgttaaaaactgtggttgttgtggagttagtaaagaaacggcgttaaaatagtaagcagccaacaaacgagtcagtggggttatttatcaactcaaatatacttttcctaaaccaaaaatcaaggcaaagaaaagaaaagattatctttccattaaaaggttgcgtgtagctaacgctcaatacgcgtcattttaacacgtgtcaaacttttattcgctattacttcacagccgtagctttctcgtgaccgagtcttcctcatcctctttgtataaatcaaaccctaaacacctcttctccttcacaaatcgataaaccaatcattctcaattctatagctctctctctctctcgcgaatttacgaatcaaaggttagatccgtgtttcttcttcttccgatcgcaattactcatcagttatttgaatttgattcaatttgttgagattagtcgatagatctcttgttgattttgattattgcgattaaggttatattatcgtttcttcttcttcgattatctgttttgtacctatcgtattgatcctctgttcttcgaatttgaatcggtttgcgtatcgttcgtgtttgtatattgaagcgatctctttgttgttttcgcaattgatctttggctttttgtgaataacagatgcagatcttcgttaagactctcaccggaaagaccatcacccttgaggtggaaagctccgacaccatcgacaacgttaaagccaagattcaggacaaggagggaatccccccggatcagcaaaggcttatcttcgccggaaagcaactcgaggatggacgtactctcgctgattacaacatccagaaggagtcgacccttcatctcgtcctcaggctccgtggtggtatgcagatcttcgttaagactcttactggaaagacgatcacactcgaggtggagagctccgacaccatcgacaacgttaaagctaagatccaagacaaggagggtattcctcccgaccagcagcgtttgatctttgccggcaagcagctcgaggacggcagaaccctcgctgattacaatatccagaaggagtccactctccacttggtgttgcgtctacgtggaggtatgcagatctttgtcaagacccttaccggcaagaccattactctagaggtggagagttccgacaccattgacaacgtcaaggctaagatccaggacaaggaaggcattcctcccgaccagcagaggttgatctttgccggcaagcagctcgaggatggtcgtaccttggctgactacaacattcagaaggagtcgactcttcatttggtgttgcgtctgcgtggtggtatgcagatctttgtgaagactcttaccggaaagaccatcacacttgaggtggagagctcggacaccattgataacgtcaaggccaaaatccaagacaaggaagggatccctccagaccagcagcgtctcatcttcgctggcaagcagcttgaggatggtcgcacccttgccgactacaacattcagaaggagtcaacccttcacttggtgttgcgtctccgtggtggcttttaagtctttgtcctctgtcgtatcatgaaacaacaatgtctgcgtttttacttccgttttctttaaaacttatcatggttggtttgtgtgtttgaggcgttataaagcctctattaaataattgatgacctctgtttcctatctcttttaagaccattcgaatgtttccatgtcgactctattcttaaatgtttggttacgttgtagatgcggtgaaacttgctgttttgcttagctgttacttctgaaagcataggggtgtttgtagcttgaagttgtcttcagaggtaggtgaggtttagggatatcccactctgcacttattggtcttctcttcaaggaccattcaagtgtaatgtggctgcttatattcaatagcttaagaagtttagtaatttggaagattcgtcattccaaaagatgttatcaatagtgtcttaggtctcgtctcaggtctttaatacgcaaaattaaaattggtgaataacagaggttggtcgagtttgcaggttaatggccgggtcattatttttgatcgactgttttatattgctgtggattatttcagtaaagaaagaactatcaaagaattcagtgaggtcttgattgaatgacgaaaccaaaaccaaaacaatgttaaaccgaggaccaggcccaacatatatgtgaattaaataaagctcaacttctacaaattaattcaatatagattaatgcatattatacaacaaaaagattcgctagttttaatataaactacattttgattggcacagatgtttatttttgtttttgtatacgttgatatttgttttacataatcaatggtttattttgttaatatttaattgtatgactttttaaatacaataatttaacttatatatattatttaataacttttatttacaagaaaaatatttttttaatagagttgataaaggtaaaataaatacaaatttaacttatatatattatttaataactttaatttacaagaaaaatatttctttttttttttaccatagactatactttttactttgataaaataagaaaaatcaacaataactttaaaaaaaaaaaagagtatcagtacatgagttagatctacatcttttgtctacagctcagagataacgatagaggaaacacaactaggaacgaatttcggcaccatcagaactgtttttgtattctaaactcagttttggtaatttggcaaattaatagaaaggattttattacaaaaatgttttaaactatgagatcagcttccatatatatcctcagagatcttaagtttcttcgcctgacggagaaggccaaacaggtgagtctagagaagatgcatcatgctcctcctcaatggcggcttcaaaaggatctttgtaacgcttcattaaaactacatttatttaactaatatatggaaccctatatatatactctcatgtttggtgtagtaagacaagttggctttccgatgtgggacaactaatgcatagtaagctcgccaaaccatattcactcactcaaatcttcaaagctatggatcaaactatcagaaacagagatgctctgtgtaaaaacgtaacaatgtgtgtgtgttccccatagattgaatatagaactatacagaactaaccagtcatcaccagttgtatagattttaaagacagagagaaaatgaaatagaaacagaatctgaatgaatatgcgtaaaa

SEQ ID NO: 61 - DNA, 20 bp, Brassica napus
  source 1..20: mol_type = genomic DNA
caaagacttaaaagccacca

SEQ ID NO: 62 - DNA, 361 bp, Arabidopsis thaliana
  source 1..361: mol_type = genomic DNA
ctttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattg

SEQ ID NO: 63 - DNA, 3695 bp, synthetic construct
  misc_feature 1..3695: note = BnUbiA09-Rfo -BnUbiA09 donor repair fragment flanked by gRNA16 target sites
  source 1..3695: mol_type = other DNA
gcgtctccgtggtggcttttaagtctttgtcctctatgcagatcttcgttaagactcttactggaaagacgatcacactcgaggtggagagctccgacaccatcgacaacgttaaagctaagatccaagacaaggagggtattcctcccgaccagcagcgtttgatctttgccggcaagcagctcgaggacggcagaaccctcgctgattacaatatccagaaggagtccactctccacttggtgttgcgtctacgtggaggtatgcagatctttgtcaagacccttaccggcaagaccattactctagaggtggagagttccgacaccattgacaacgtcaaggctaagatccaggacaaggaaggcattcctcccgaccagcagaggttgatctttgccggcaagcagctcgaggatggtcgtaccttggctgactacaacattcagaaggagtcgactcttcatttggtgttgcgtctgcgtggtggtatgcagatctttgtgaagactcttaccggaaagaccatcacacttgaggtggagagctcggacaccattgataacgtcaaggccaaaatccaagacaaggaagggatccctccagaccagcagcgtctcatcttcgctggcaagcagcttgaggatggtcgcacccttgccgactacaacattcagaaggagtcaacccttcacttggtgttgcgtctccgtggtggcatgttggctagggtttgtggattcaagtgttcttcttctcctgctgagtctgcggctagattgttctgtacgagatcgattcgtgatactctggccaaggcaagcggagagagttgcgaagcaggttttggaggagagagtttgaagctgcaaagtgggtttcatgaaatcaaaggtttagaggatgcgattgatttgttcagtgacatgcttcgatctcgtcctttaccttctgtggttgatttctgtaaattgatgggtgtggtggtgagaatggaacgcccggatcttgtgatttctctctatcagaagatggaaaggaaacagattcgatgtgatatatacagcttcaatattctgataaaatgtttctgcagctgctctaagctcccctttgctttgtctacatttggtaagatcaccaagcttggactccaccctgatgttgttaccttcaccaccctgctccatggattatgtgtggaagatagggtttctgaagccttggatttttttcatcaaatgtttgaaacgacatgtaggcccaatgtcgtaaccttcaccactttgatgaacggtctttgccgcgagggtagaattgtcgaagccgtagctctgcttgatcggatgatggaagatggtctccagcctacccagattacttatggaacaatcgtagatgggatgtgtaagaagggagatactgtgtctgcactgaatctgctgaggaagatggaggaggtgagccacatcatacccaatgttgtaatctatagtgcaatcattgatagcctttgtaaagacggacgtcatagcgatgcacaaaatcttttcactgaaatgcaagagaaaggaatctttcccgatttatttacctacaacagtatgatagttggtttttgtagctctggtagatggagcgacgcggagcagttgttgcaagaaatgttagaaaggaagatcagccctgatgttgtaacttataatgctttgatcaatgcatttgtcaaggaaggcaagttctttgaggctgaagaattatacgatgagatgcttccaaggggtataatccctaatacaatcacatatagttcaatgatcgatggattttgcaaacagaatcgtcttgatgctgctgagcacatgttttatttgatggctaccaagggctgctctcccaacctaatcactttcaatactctcatagacggatattgtggggctaagaggatagatgatggaatggaacttctccatgagatgactgaaacaggattagttgctgacacaactacttacaacactcttattcacgggttctatctggtgggcgatcttaatgctgctctagaccttttacaagagatgatctctagtggtttgtgccctgatatcgttacttgtgacactttgctggatggtctctgcgataatgggaaactaaaagatgcattggaaatgtttaaggttatgcagaagagtaagaaggatcttgatgctagtcaccccttcaatggtgtggaacctgatgttcaaacttacaatatattgatcagcggcttgatcaatgaagggaagtttttagaggccgaggaattatacgaggagatgccccacaggggtatagtcccagatactatcacctatagctcaatgatcgatggattatgcaagcagagccgcctagatgaggctacacaaatgtttgattcgatgggtagcaagagcttctctccaaacgtagtgacctttactacactcattaatggctactgtaaggcaggaagggttgatgatgggctggagcttttctgcgagatgggtcgaagagggatagttgctaacgcaattacttacatcactttgatttgtggttttcgtaaagtgggtaatattaatggggctctagacattttccaggagatgatttcaagtggtgtgtatcctgataccattaccatccgcaatatgctgactggtttatggagtaaagaggaactaaaaagggcagtggcaatgcttgagaaactgcagatgagtatggtatgtaagtttctgttcagtctatgtattttttatataaacaagaatgtatacattcttttgtgtgtagcttcagattgatgatacacgttctggaattaaccattggtttggttttgcattgtaggatctatcatttgggggatgagtctttgtcctctgtcgtatcatgaaacaacaatgtctgcgtttttacttccgttttctttaaaacttatcatggttggtttgtgtgtttgaggcgttataaagcctctattaaataattgatgacctctgtttcctatctcttttaagaccattcgaatgtttccatgtcgactctattcttaaatgtttggttacgttgtagatgcggtgaaacttgctgttttgcttagctgttacttctgaaagcataggggtgtttgtagcttgaagttgtcttcagaggtaggtgaggtttagggatatcccactctgcacttattggtcttctcttcaaggaccattcaagtgtaatgtggctgcttatattcaatagcttaagaagtttagtaatttggaagattcgtcattccaaaagatgttatcaatagtgtcttaggtctcgtctcaggtctttaatacgcaaaattaaaattggtgaataacagaggttggtcgagtttgcaggttaatggccgggtcattatttttgatcgactgttttatattgctgtggattatttcagtaaagaaagaactatcaaagaattcagtgaggtcttgattgaatgacgaaaccaaaaccaaaacaatgttaaaccgaggaccaggcccaacatatatgtgaattaaataaagctcaacttctacaaattaattcaatatagattaatgcatattatacaacaaaaagattcgctagttttaatatgcgtctccgtggtggcttttaagtctttgtcctct

SEQ ID NO: 64 - DNA, 732 bp, Cauliflower mosaic virus
  source 1..732: mol_type = genomic DNA
acgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggac

SEQ ID NO: 65 - DNA, 4212 bp, synthetic construct
  misc_feature 1..4212: note = SV40 NLS-Cas9-Nucleoplasmin NLS
  source 1..4212: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggcacatcctag

SEQ ID NO: 66 - DNA, 680 bp, Triticum aestivum
  source 1..680: mol_type = genomic DNA
atgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtgg

SEQ ID NO: 67 - DNA, 684 bp, Triticum aestivum
  source 1..684: mol_type = genomic DNA
atgcagatcttcgtgaagacccttaccggcaagaccatcacccttgaggtcgagtcctctgacaccatcgacaacgtcaaggcaaagatccaggacaaggagggcatccccccggaccagcagcgtctcatctttgctggcaagcagctcgaggatggccgcacccttgcggactacaacatccagaaggagtccaccctccacctggttctcaggctccgtggtggtatgcagatatttgtcaagaccctcactggcaaaaccatcaccctcgaggttgagtcctcagacaccattgataatgtgaaggccaagatccaggacaaggagggtatcccaccggaccagcagcgtctcatctttgctggcaagcagctcgaggatggccgcacccttgcagactacaacatccagaaggagtccactctccacttggtgctcaggctccgtggtggtatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggc

SEQ ID NO: 68 - DNA, 740 bp, Triticum aestivum
  source 1..740: mol_type = genomic DNA
cagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcag

SEQ ID NO: 69 - DNA, 650 bp, Triticum aestivum
  source 1..650: mol_type = genomic DNA
gacgggattgctgtggggtcggttcgtcttagttctgcttggttgttattgtggatttttgattatgtaccgctcatgagcctgatatgttgaaggatacaccttcgtggtgttattgggatgcgatgcgattgctgttggttcgcttcatgtatctgttatagttagctactacagtagtttctgattttgtagatcctatgatccgtggaaggatacgcctgtggtttgctagcttaatagttcagcttttgtagttgcttgtagcgtccctagttgagcatcgaatttaactcttaaattcatgtcggcatgtcggtttgccgacattactgttcgtttctaggcttcttgacaagcttctcagtacaactagattacataagctcccactgaagctaaagattgctctggtagatatgctaatatattattctgctaaaatttagcactggatggtctgtcaatccattggcttgttctaggggccagctcctgggcagtttgctgtcagctctctagctgatttcatctctgttgcaatttcatctttgtagcatggtggccatctggttcatttattgtttgacgtactgcagatatccctgtacgctaacttactctggtttctgtctgctttgtatttacag

SEQ ID NO: 70 - protein, 14 aa, Triticum aestivum
KESTLHLVLRLRGG

SEQ ID NO: 71 - DNA, 1554 bp, Lolium rigidum
  source 1..1554: mol_type = genomic DNA
atggataaggcatacattgccatcctctcctgcgccttcctcttcctggtccactacgttctcggcaaggtcagccatggcaggcgcggcaagaagggcgccgtgcagctgccgccgagccccccggccatcccgttcatcggccacctccacctcgtggagaagcccatccacgccacgatgtgccgcctcgccgcgcgcctcggcccggtcttctcgctgcgcctcggatcccgccgcgccgtggtggtgtcctcgtccgagtgcgccagggagtgcttcacggagcacgacgtgaccttcgccaaccggcccaagttcccgtcgcagctcctcgcgtccttcaacggcacggcgctcgtcacgtccagctacggcccgcactggcgcaacctccggcgggtcgccaccgtgcagctgctctccgcgcaccgcgtcgcctgcatgtccggcgtcatcgccgcagaggtccgcgccatggcacgccggctgttccatgccgccgaggcgtcccccgacggcgccgcgcgggtccagctgaagcggaggctgttcgagctctccctcagcgtgctcatggagaccatcgcgcagaccaaggccacccgctcggaggccgacgccgacacggacatgtccgtggaggcgcaggagttcaaggaggtggtggacaagctcatcccgcacctcggcgccgccaacatgtgggactacctgccggtgatgcggtggttcgacgtcttcggcgtgaggaacaagatcttgcacgctgtgagccggagggacgcgttcctgcggcgactcatcgatgccgaacgccggaggctggccgacggcggcagcgatggcgataagaagagcatgatcgccgtgctgctcactctgcagaagacggagccaaaggtctacaccgataccatgatcacggctctctgcgcgaatttatttggggccgggacggagaccacgtcaaccacgacagagtgggcgatgtcgctcctgctgaaccacccggcagcgctgaagaaggcccaggccgagatcgacgcgtcagtcgggacctcccgtctggtgtccgtcgacgacgtgcccagtctcgcctacctgcagtgcatcgtcagcgagacgctccgcctctacccggcggcgccgctgctgcttccacacgagtcctccgcggactgcaaggttggcggctacaacgtgccggccgacacgatgctgatcgtgaacgcttacgccattcaccgggacccggcggcgtgggagcacccgctggagtttaggccggacaggttcgaggacggcaaggccgagggtctgttcatgataccgttcgggatggggcggcggaggtgccccggggagacgctggcgctgcggacgatcggaatggtcctggcgacgctggtgcagtgcttcgactgggaaccggtggacggcgtgaaggtggacatgacggaggggggagggttcaccatcccaaaggccgtgccgttggaggccgtgtgcaggccgcgcgcggtcatgcgcgacgtgcttcagaacctctaa

SEQ ID NO: 72 - protein, 517 aa, Lolium rigidum
MDKAYIAILSCAFLFLVHYVLGKVSHGRRGKKGAVQLPPSPPAIPFIGHLHLVEKPIHATMCRLAARLGPVFSLRLGSRRAVVVSSSECARECFTEHDVTFANRPKFPSQLLASFNGTALVTSSYGPHWRNLRRVATVQLLSAHRVACMSGVIAAEVRAMARRLFHAAEASPDGAARVQLKRRLFELSLSVLMETIAQTKATRSEADADTDMSVEAQEFKEVVDKLIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTEWAMSLLLNHPAALKKAQAEIDASVGTSRLVSVDDVPSLAYLQCIVSETLRLYPAAPLLLPHESSADCKVGGYNVPADTMLIVNAYAIHRDPAAWEHPLEFRPDRFEDGKAEGLFMIPFGMGRRRCPGETLALRTIGMVLATLVQCFDWEPVDGVKVDMTEGGGFTIPKAVPLEAVCRPRAVMRDVLQNL

SEQ ID NO: 73 - DNA, 1892 bp, Zea mays
  source 1..1892: mol_type = genomic DNA
tatgtgtgtatatatatgtgcagccatgcaggatggtgccagcgccacatacaaacaccaaagcagcgatcgatcgatcaatcaaagctcgtgggccagaacatctcctgctccacgaccacgaccatggataaggcctacgtggccgtgctctccttcgccttcctcttcgtgatccactacctcgtgggacgtgctggccgcaagggcaatggcaagggcaagggaacgcagcggttgcctcccagccctccggccgtcccgttcctcggccacctccacctcgtcaagacgccgttccacgaggcgctggccggcctcgccgcgcgccacgggccggtgttctccatgcgcatggggtcccgccgcgcgctggtcgtgtcctcgcccgagtgcgccaaggagtgcttcacggagcacgacgtggtgttcgccaaccggccgaggttcgccacgcaggacctcgtgtccttcgggggcgccgcgctcgccgcggccagctacgggccctactggcgcaacctccggcgcgtggccaccgtgcagctcctctccgcgcaccgcgtcgcctgcatgtccgccgtcgtcgccgccgaggtgcgcgccatggcgcgccgcatgggccgcgcggccgcggcggcgcccggcggcgccgcgcgcgtccagctcaagcggaggctgttcgaggtctccctcagcgtgctcatggagaccatcgcgcgcaccaagacgtcccgcgccgaggcggacgccgactcggacatgtcgcccgaggcgcacgaattcaagcagatcgtcgacgagatcgtgccgcacctcggcacggccaacctgtgggactacctgccggtgctgcggtggctcgacgtgttcggcgtgaggaacaagatcacggccgctgtgggcaggagggacgcgttcctgcggcggctcatcgacgcggagcggcggaggctggacgacggcggcggcgacagcgacagcgacaagaagagcatgatagccgtgctgctctcgttgcagaagtcagagccggaggtgtacacggataccatgatcatggcactttgcgggaacttatttggcgccggaacggagaccacgtcgaccacgacagaatgggccatgtcgctcctcctgaaccacccggaggcgctgaagaaggcacaggcagagatcgacgcagtcgtgggcacctcccgcctactcgccgcagaggacgtgccccgactcggctacctgcaccgcgtcatcagcgagaccctgcgcatgtacccggccgccccgctgctcctgccgcacgagtcgtcggcggactgcaaggtcggcggctacgacgtggcccgcggcacgctgctcatcgtgaacgcgtacgccatccacagggacccgctggtgtgggaggacccggacgagttcaggccggagcggttcgaggacggcaaggccgaggggcggctcctgatgccgttcgggatggggcggcgcaagtgccctggggagaccctcgcgctgcggaccatcagcctggtgctcggcacgctgatccagtgcttcgactgggaccgagtcgacggcctcgagatcgacatggccgcgggcggcgggctcactctgcccagggccgtcccgttggaggccacgtgcaagcctcgtgcagctgtgcgccatcttcttctggagctctgaatctgatgctgtagtaacatcttggcatgcatatgatccctagtatgctttgcctgatggtgtgtgctaaactaaataagcttgagtgcgtaataccctcgtgcgtgttatcttctgtaaaatgtgttgtacagtcgtttgcgtgaacaatcaatcagtagttggttcacagtatggtttatagcaaattgtggtcttctgca

SEQ ID NO: 74 - protein, 557 aa, Zea mays
MCSHAGWCQRHIQTPKQRSIDQSKLVGQNISCSTTTTMDKAYVAVLSFAFLFVIHYLVGRAGRKGNGKGKGTQRLPPSPPAVPFLGHLHLVKTPFHEALAGLAARHGPVFSMRMGSRRALVVSSPECAKECFTEHDVVFANRPRFATQDLVSFGGAALAAASYGPYWRNLRRVATVQLLSAHRVACMSAVVAAEVRAMARRMGRAAAAAPGGAARVQLKRRLFEVSLSVLMETIARTKTSRAEADADSDMSPEAHEFKQIVDEIVPHLGTANLWDYLPVLRWLDVFGVRNKITAAVGRRDAFLRRLIDAERRRLDDGGGDSDSDKKSMIAVLLSLQKSEPEVYTDTMIMALCGNLFGAGTETTSTTTEWAMSLLLNHPEALKKAQAEIDAVVGTSRLLAAEDVPRLGYLHRVISETLRMYPAAPLLLPHESSADCKVGGYDVARGTLLIVNAYAIHRDPLVWEDPDEFRPERFEDGKAEGRLLMPFGMGRRKCPGETLALRTISLVLGTLIQCFDWDRVDGLEIDMAAGGGLTLPRAVPLEATCKPRAAVRHLLLEL

SEQ ID NO: 75 - DNA, 1851 bp, Zea mays
  source 1..1851: mol_type = genomic DNA
aaaccaccaactcgcgatccatcgatttgcagaacaccacccaacagccaaaccatcactcgatcggtagacatggataaggcctacatcgccgccctctccgccgccgccctcttcttgctccactacctcctgggccggcgggccggcggcgagggcaaggccaaggccaagggctcgcggcggcggctcccgccgagccctccggcgatcccgttcctgggccacctccacctcgtcaaggccccgttccacggggcgctggcccgcctcgcggcgcgccacggcccggtgttctccatgcgcctggggacccggcgcgccgtggtcgtgtcgtcgccggactgcgccagggagtgcttcacggagcacgacgtgaacttcgcgaaccggccgctgttcccgtcgatgcggctggcgtccttcgacggcgccatgctctccgtgtccagctacggcccgtactggcgcaacctgcgccgcgtcgccgccgtgcagctcctctccgcgcaccgcgtcgggtgcatggcccccgccatcgaagcgcaggtgcgcgccatggtgcggaggatggaccgcgccgccgcggccggcggcggcggcgtcgcgcgcgtccagctcaagcggcggctgttcgagctctccctcagcgtgctcatggagaccatcgcgcacaccaagacgtcccgcgccgaggccgacgccgactcggacatgtcgaccgaggcccacgagttcaagcagatcgtcgacgagctcgtgccgtacatcggcacggccaaccgctgggactacctgccggtgctgcgctggttcgacgtgttcggcgtgaggaacaagatcctcgacgccgtgggcagaagggacgcgttcctggggcggctcatcgacggggagcggcggaggctggacgctggcgacgagagcgaaagtaagagcatgattgcggtgctgctcactctgcagaagtccgagccagaggtctacactgacactgtgatcactgctctttgcgcgaacctattcggcgccggaacggagaccacgtccaccacgacggaatgggccatgtcactgctgctgaaccaccgggaggcgctcaagaaggcgcaggccgagatcgacgcggcggtgggcacctcccgcctggtgaccgcggacgacgtgccccacctcacctacctgcagtgcatcgtcgacgagacgctgcgcctgcacccggccgcgccgctgctgctgccgcacgagtccgccgcggactgcacggtcggcggctacgacgtgccgcgcggcacgatgctgctggtcaacgtgcacgcggtccacagggaccccgcggtgtgggaggacccggacaggttcgtgccggagcggttcgagggcgccggcggcaaggccgaggggcgcctgctgatgccgttcgggatggggcggcgcaagtgccccggggagacgctcgcgctgcggaccgtcgggctggtgctcgccacgctgctccagtgcttcgactgggacacggttgatggagctcaggttgacatgaaggctagcggcgggctgaccatgccccgggccgtcccgttggaggccatgtgcaggccgcgtacagctatgcgtggtgttcttaagaggctctgaaaacctcatggatcgaattgctggcatcgtctgaagggtgtatgacgtagcttccgagttccgagcatatatattcacttgccttgtactagttgattttcgccgagtgtatggaatggattttcttttttttcttgcaatggatgtgaattttgtttttctcgacgttacaagaagtgaatcaacctagcttctgaaaaaaaaaaaaaaaaa

SEQ ID NO: 76 - protein, 521 aa, Zea mays
MDKAYIAALSAAALFLLHYLLGRRAGGEGKAKAKGSRRRLPPSPPAIPFLGHLHLVKAPFHGALARLAARHGPVFSMRLGTRRAVVVSSPDCARECFTEHDVNFANRPLFPSMRLASFDGAMLSVSSYGPYWRNLRRVAAVQLLSAHRVGCMAPAIEAQVRAMVRRMDRAAAAGGGGVARVQLKRRLFELSLSVLMETIAHTKTSRAEADADSDMSTEAHEFKQIVDELVPYIGTANRWDYLPVLRWFDVFGVRNKILDAVGRRDAFLGRLIDGERRRLDAGDESESKSMIAVLLTLQKSEPEVYTDTVITALCANLFGAGTETTSTTTEWAMSLLLNHREALKKAQAEIDAAVGTSRLVTADDVPHLTYLQCIVDETLRLHPAAPLLLPHESAADCTVGGYDVPRGTMLLVNVHAVHRDPAVWEDPDRFVPERFEGAGGKAEGRLLMPFGMGRRKCPGETLALRTVGLVLATLLQCFDWDTVDGAQVDMKASGGLTMPRAVPLEAMCRPRTAMRGVLKRL

SEQ ID NO: 77 - DNA, 1551 bp, Triticum aestivum; identical to SEQ ID NO: 41
  source 1..1551: mol_type = genomic DNA

SEQ ID NO: 78 - protein, 516 aa, Triticella sp.; identical to SEQ ID NO: 42

SEQ ID NO: 79 - protein, 644 aa, Triticum aestivum
MAAATSPAVAFSGATAAAMPKPARHPLPRHQPVSRRALPARVVRCCAASPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDERVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 80 - protein, 644 aa, Triticum aestivum
MAAATSPAVAFSGATAAAMPKPARHPLPRHQPVSRRALPARVVRCCAASPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENESEIYPDFVTIAKGFNVPAVRVTKKSEVTAAIKKMLETPGPYLLDIIVPHQEHVLPMIPNGGAFKDMIMEGDGRTSY

SEQ ID NO: 81 - protein, 647 aa, Triticum aestivum
MAAATSPAVAFSGAAAAAAAMPKPARQPLPRHQPASRRALPARVVRCCAAPPAAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPIWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDERVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 82 - protein, 647 aa, Triticum aestivum
MAAATSPAVAFSGAAAAAAAMPKPARQPLPRHQPASRRALPARVVRCCAAPPAAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPIWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENESEIYPDFVTIAKGFNVPAVRVTKKSEVTAAIKKMLETPGPYLLDIIVPHQEHVLPMIPNGGAFKDMIMEGDGRTSY

SEQ ID NO: 83 - protein, 644 aa, Triticum aestivum
MAAATSPAVAFSGAAAAAIPKPARQPLPRHQPASRRALPARIVRCCAASPAATSVAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDERVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENEGEIYPDFVTIAKGFNVPAVRVTKKSEVTAAIKKMLETPGPYLLDIIVPHQEHVLPMIPSGGAFKDMIMEGDGRTSY

SEQ ID NO: 84 - protein, 644 aa, Triticum aestivum
MAAATSPAVAFSGAAAAAIPKPARQPLPRHQPASRRALPARIVRCCAASPAATSVAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQLEDRFYKANRAHTYLGNPENEGEIYPDFVTIAKGFNVPAVRVTKKSEVTAAIKKMLETPGPYLLDIIVPHQEHVLPMIPNGGAFKDMIMEGDGRTSY

SEQ ID NO: 85 - DNA, 5442 bp, synthetic construct
  source 1..5442: mol_type = other DNA
  misc_feature 1..5442: note = TaUbi7DL_promoter::TaUbi7DL_cds::TaALS1_chr6D_cds:: TaUbi7DL_terminator
gagttcacgggaaatgtcacgaaaagccacgacgattctctgtatccttcatacactgagctccgaaaagcgatgccccgtgaaatagtgtctcgtcgaattttattttcttctttccgggaggttttgtgattaagaatttaaagcagataagaggatgagagcgaaagcaacatccataacgcgacgtgtgggacgaaaagtcatgagggacgcgtccttcccgttcctcaccgtcatataacgtcggcgtatattacatggacgtcgcgctcgggtggggatgacaagcaggtgctggtgcctgtttttcacacggttgtcgtgctttttaatttaaataattgtgcatttgaattttcgagagaaagtaatgggactgactacttgtgtgttgcctgaaaaaaagatagttttacttcattcctttgtcgacggcttccttatgctccatagggtgcggcgagtaggccggcccggcaggacgagctgcaggggcgtgcgggcgcggcgagcaggccggcggggtcgacgcaactgaagcagaggacctcggcgggcgacatggcaacggccgtgtgagcaggccggcgtggtggtgtcggtgcggtggagggagatgaggtggcacatggggaataagagacaaaagtgctagggggggctggaagaatcttcatcccaatgtaaaattttcgccgaggccttcttgggaggccgaaaaaccgccttctgaggggcacgacggccggagatgctctcacgcgtgagtgagtgcccattgtgtgtcacgctcacggaccacacacaccaacggcgtgagccgacgcgtccaactccaacggccaccaaccagccggctttccaaccagccaaatccccaacggcagcaaacggcgtcacacctcccatatccttgccctttttgcgtggtgggggacgaactccgcgaccgcctctccctcctcttcctatccccacacgtcacgtcaggcgtgccgtagcgcttcttccttctctccctcccgtcggatccggctataaattacccgccccctcccgtctcctccccacacatccgatccgatccaatccagcccccctccgatctcgccgagaagtctccgattgaagcgaagcgaagccaaggctctctcctcctcctcctaaggtatgcgatgcgaggctacgatttcttctccgctctctcgtgcgtgcgtgaggactgcttgatcggttattgttggttcccctcgattcctgttagtcgtatggatttggagtagatctgtgggtgggtcgaggcgttattgctgttgatcgattgcagctcgttcggttagccgtggttgattaggcctgattgtagttaataccatctctcgtgattatgccatgattttcgagtagatctgccgattatgacgggattgctgtggggtcggttcgtcttagttctgcttggttgttattgtggatttttgattatgtaccgctcatgagcctgatatgttgaaggatacaccttcgtggtgttattgggatgcgatgcgattgctgttggttcgcttcatgtatctgttatagttagctactacagtagtttctgattttgtagatcctatgatccgtggaaggatacgcctgtggtttgctagcttaatagttcagcttttgtagttgcttgtagcgtccctagttgagcatcgaatttaactcttaaattcatgtcggcatgtcggtttgccgacattactgttcgtttctaggcttcttgacaagcttctcagtacaactagattacataagctcccactgaagctaaagattgctctggtagatatgctaatatattattctgctaaaatttagcactggatggtctgtcaatccattggcttgttctaggggccagctcctgggcagtttgctgtcagctctctagctgatttcatctctgttgcaatttcatctttgtagcatggtggccatctggttcatttattgtttgacgtactgcagatatccctgtacgctaacttactctggtttctgtctgctttgtatttacagatgcagatcttcgtcaagaccctcaccggcaagaccatcaccctggaggttgagtcctcggacactattgacaatgttaaggccaagatccaggacaaggagggcattcccccggaccagcagcgtcttatctttgccggcaagcagcttgaggatggccgcacccttgcggattacaacatccagaaggaatccaccctccaccttgtcctccgccttcgtggtggcatgcaaatctttgtcaagaccctcaccggcaagaccatcaccctggaggttgagtcctcggacaccattgacaatgtgaaggccaagatccaggacaaggagggcatccccccggaccagcagcgcctgatctttgccggcaagcagctcgaggatggtcgcacccttgcggattacaacatccagaaggagtccacccttcacttggtgctcaggctccgtggtggcatgcagatatttgtgaagaccctcaccggcaagaccatcaccctggaggtggagtcatctgacaccattgacaacgtgaaggcgaagatccaggacaaggagggcattcccccggaccagcagcgtctcatctttgccggtaagcagcttgaggatggccgcaccttggcggactacaacatccagaaggagtccacccttcacttggtgctccgtctccgtggcggtatggccgccgccacctcccccgccgtcgcattctcgggcgccaccgccgccgccatgcccaaacccgcccgccatcctctcccgcgccaccagcccgtctcgcgccgcgcgctccccgcccgcgtcgtcaggtgttgcgccgcgtcccccgccgccacctccgccgcgcctcccgcaaccgcgctccggccctggggcccgtccgagccccgcaagggcgccgacatcctcgtcgaggcgctcgagcgctgcggcatcgtcgacgtcttcgcctaccccggcggcgcctccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgccgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttccttgcatcctctggccgcccggggccggtgctagttgatatccccaaggacatccagcagcagatggctgtgcccgtctgggacactccaatgagtttgccagggtacatcgcccgcctgcccaagccaccatctactgaatcgcttgagcaggtcctgcgtctggttggcgagtcacggcgcccaattctgtatgttggtggtggctgcgctgcgtctggcgaggagttgcgccgctttgttgagcttactgggattccagttacaactactctgatgggccttggcaacttccccagcgacgacccactgtctctgcgcatgcttgggatgcatggcactgtgtatgcaaattatgcagtagataaggctgacctgttgctcgcatttggtgtgcggtttgatgatcgtgtgactgggaaaatcgaggcttttgcaagcaggtccaagattgtgcacattgacattgacccagctgagattggcaagaacaagcagccacatgtctccatttgtgcagatgttaagcttgctttacaggggttgaatgatctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctgggaatggtggtgcagtgggaggataggttttacaaggccaatcgggcgcacacataccttggcaacccagaaaatgagagtgagatatatccagattttgtgacgattgctaaaggattcaacgttccagcagttcgagtgacgaagaagagtgaagtcactgcagcaatcaagaagatgcttgagaccccagggccatacttgttggatatcatagtcccgcatcaggagcacgtgctgcctatgatcccaagcggtggtgctttcaaggacatgatcatggagggtgatggcaggacctcgtactgaaaccagtaagctcctggccatggagctgcttctgtctctgggttcacaagtctcggtgtctccggtatcctccaatggagtctggtctgtgtctgtcgttgcctgaactgtctttgtttctgtaccatactgtgatgcagtgttatcgtttgtatcttcaaacttctgctggtgtggagcagctttggtgaactatgaataagtgagcgcttaatttggtccataattttgtttgtcttgcctgatgtttgctgtgttttatgtggtttcttgcgatctttctggtgcatttgatctgctctgttggaacatgttttctggttggtgacctctatgcgtgtgttcgaactgattttgtttgtatactaagtgccaactttgatttctggcctgtagaatcccttaattctcaaattttctgtgaaagatatgtggaatcacctgtgctgcaactggtctttggcccattttacgcaaacgtacgcgtgttcttctggctgatcttgtgcttttgttggctgcatgacctcgcatagcgcgttgattttgtgtcatcgcctgtgttgtggagctgctgttgcaaagtgggtggacgtgggccgctacaagggtctagcttgccacaatgagacatgagctgaagttgcaacatgaggtcttgttacaattgcaacaagtgaaagagagaataggatcgtgagaccagactggtcgtgacccggccgatctttttcaaagatcagccggccg

(19) United States
(12) Patent Application Publication
(10) Pub. No.: US 2025/0263733 A1
(43) Pub. Date: Aug. 21, 2025

(54) TARGETED GENE INTEGRATION IN PLANTS

(71) Applicant: LIMAGRAIN EUROPE, SAINT-BEAUZIRE (FR)

(72) Inventors: Wyatt PAUL, CHAPPES (FR); Pascual PEREZ, CHANONAT (FR); Jacques ROUSTER, MIREFLEURS (FR)

(21) Appl. No.: 18/702,467

(22) PCT Filed: Oct. 19, 2022
(86) PCT No.: PCT/EP2022/079130

§ 371 Date: Apr. 18, 2024

(30) Foreign Application Priority Data
Oct. 19, 2021 (EP) 21306460.3

(51) Int. Cl. C12N 15/82; C12N 9/22; C12N 15/11; C12Q 1/6895

(52) U.S. Cl. CPC C12N 15/8213; C12N 9/226; C12N 15/111; C12N 15/8274; C12N 15/8286; C12N 15/8289; C12Q 1/6895; C12N 2310/20; C12Q 2600/13

(57) ABSTRACT

The present invention relates to a vector suitable for a targeted integration of at least one gene of interest in 5′ or 3′ of a polyubiquitin gene in a plant. The present invention also relates to the use of said vector in a method for targeted insertion of at least one gene of interest in a plant genome and to a plant cell or plant tissue obtained by transformation with said vector. The present invention further relates to a method of identifying a plant having at least one gene of interest inserted in 5′ or 3′ of a polyubiquitin gene.

### FIELD OF THE INVENTION

The present invention concerns the targeted integration of genes of interest in plants.

### BACKGROUND OF THE INVENTION

Genetically modified plants often require constitutive and high-level transgene expression in order to possess a new desired agronomic trait. This is most often achieved by the transformation of the plant with one or several transgene cassette(s) comprising of a constitutive promoter linked to the gene of interest (GOI) causing the new trait, and a plant transcript polyadenylation sequence. Transgenic transformation events, created using biolistic or Agrobacterium transgene delivery, are usually randomly integrated into the plant genome.

However, this procedure is not optimal for several reasons. First, the transgene can integrate into endogenous genes, potentially causing a loss of function or altered functionality of these endogenous genes, leading to undesirable phenotypes, sometimes pleiotropic. Secondly, the level of expression of the transgene can be modulated by the surrounding genomic environment. Each transgenic event may have a different level of expression and spatial-temporal expression profile. Thirdly, often transgene events have multiple insertions of the transgene; these are usually discarded. Fourthly, random transgene insertions might create novel open reading frames that may prevent or slow deregulation of the transgenic event. Together, these factors result in the need to make 1000 or more primary plant transformants in order to arrive at a commercially and agronomically acceptable genetically modified event.

Positioning of a transgene at a defined place in the genome (named a Landing Pad) addresses the concerns outlined above, however an ideal genome location for transgene insertion needs to be identified and tested. Positioning a transgene outside of an endogenous gene is not ideal since the performance of the transgene in terms of expression and stability of expression is hard to predict. Positioning within an endogenous gene would reduce this uncertainty, but as described above might disrupt the function of the endogenous gene. WO2013169/802 for example discloses methods of nuclease-mediated integration of transgenes.

WO2018/005589 discloses different methods of insertion of a gene of interest in a plant genome. When the insertion of a gene of interest is to be performed in the 3′ region of a gene sequence comprising a stop codon, this document discloses that the insertion must occur before the stop codon. A T2A sequence has to be introduced alongside the gene of interest in order to liberate the protein of interest from the fusion protein obtained after the insertion.

Hondred et al. (1999, Plant Physiol. 119:713-24. doi: 10.1104/pp. 119.2.713.) have demonstrated via transgenesis in tobacco that β-glucuronidase (GUS), fused translationally to the 3′ end of polyubiquitin, is highly expressed and processed by an endogenous protease liberating GUS.

There is still a need for improved methods allowing targeted gene integrations in plants, wherein the gene of interest is efficiently expressed.

### DESCRIPTION OF THE INVENTION

The Inventors have surprisingly found that inserting a gene of interest (GOI) in 5′ or in 3′ of an endogenous polyubiquitin gene in a plant, such that the GOI is expressed as a polyubiquitin: GOI encoded protein fusion, allows an efficient targeted gene insertion. The fusion protein is then processed by endogenous ubiquitin proteases to release the protein encoded by the GOI and ubiquitin monomers.

Advantageously, GOI insertion according to the invention has no effect on the functionality of the polyubiquitin gene and allows an efficient and stable expression of the GOI.

Besides, the advantages of a polyubiquitin gene as a Landing Pad are that the expression of the GOI is directed by a strong and constitutive endogenous polyubiquitin promoter. The expression of a GOI under this strong and constitutive promoter is also within the scope of the invention. All GOI insertion events at polyubiquitin should have similar levels of expression.

Interestingly, if the short amino acid target site cleaved by the polyubiquitin protease is introduced between several GOI elements, multiple GOIs can be expressed.

Furthermore, targeting of the protein of interest to various cellular compartments (cytosol, mitochondria or plastids) is not affected by this method and correct expression of GOI in one or more cellular compartments are under the scope of the invention.

A first object of the invention is thus a vector
suitable for a targeted integration of at least one gene of interest in 5′ or 3′ of a polyubiquitin gene in a plant, wherein said vector comprises a repair DNA comprising from 5′ to 3′:
   a first gRNA target,
   a left ubiquitin-like region,
   at least one gene of interest,
   a right ubiquitin-like region, and
   a second gRNA target.

The vector as defined above may further comprises:
at least one CRISPR-Cas endonuclease expression cassette and/or
at least one gRNA expression cassette, preferably encoding a gRNA able to recognize a region in 3′ or 5′ of the polyubiquitin gene, preferably a single gRNA expression cassette.

These two or three cassettes may be on the same or in different vectors.

The gene of interest to be integrated may for example be selected from the group consisting of a herbicide tolerance gene, an insect resistance gene, a fungal resistance gene, a bacterial resistance gene, a stress resistance gene, a gene involved in reproductive capability, a gene involved in performance in the fields, a gene involved in performance in an industrial process and a gene involved in nutritional value of a plant.

Said gene of interest may for example be selected from the group consisting of a BAR gene, ALS gene, GS gene, cyt P450 gene, RFL29a gene, RFL79 gene, Rfo gene, Cry1Ac gene and RCA-Cry1 Ac gene.

Another object of the invention is a plant cell or plant tissue which is transformed with the vector as defined above.

Another object of the invention is a plant cell or plant tissue comprising at least one gene of interest inserted in 5′ or in 3′ of the polyubiquitin gene, obtained by transformation with the vector as defined above.

Said plant cell or plant tissue may for example be a protoplast, apical meristem, cotyledon, embryo, pollen and/or microspores.

Another object of the invention is a plant comprising at least one gene of interest inserted in 5′ or in 3′ of the polyubiquitin gene, obtained by transformation with the vector as defined above.

The plant, plant cell or plant tissue comprises at least one polyubiquitin gene.

Said plant may be a monocotyledon or a dicotyledon.

Another object of the invention is a progeny plant of a plant as defined above, wherein said progeny plant comprises at least one gene of interest inserted in 5′ or in 3′ of the polyubiquitin gene.

Another object of the invention is a method for targeted insertion of at least one gene of interest in 5′ or 3′ of a polyubiquitin gene in a plant genome, comprising:
a. the transformation of a plant cell or plant tissue with at least one vector as defined above, to obtain a transformed plant cell or plant tissue, and
b. the regeneration of the plant from the transformed plant cell or plant tissue.

In the method as defined above, at least one CRISPR-Cas endonuclease expression cassette may be provided by said vector or in a separate vector and wherein at least one gRNA expression cassette is provided by said vector or in a separate vector.

Another object of the invention is a method for expressing at least one protein of interest in a plant, comprising the steps of the method as defined above for targeted insertion of at least one gene of interest in 5′ or 3′ of a polyubiquitin gene in a plant genome, wherein said gene of interest codes for said protein of interest.

Another object of the invention is the use of the vector as defined above, for expressing at least one gene of interest in a plant, in a plant cell or in a plant tissue.

Another object of the invention is a method of identifying a plant comprising at least one gene of interest inserted in 5′ or 3′ of a polyubiquitin gene, wherein said method comprises:
extracting the DNA and/or RNA of a plant,
detecting the presence of a DNA comprising at least one gene of interest inserted in 5′ or 3′ of a polyubiquitin gene and/or the presence of a RNA transcript from said DNA, and
optionally, detecting the presence of a protein encoded by said at least one gene of interest.

### Plant

The plant for use according to the invention comprises at least one polyubiquitin gene.

The plant as defined above may be a monocotyledon or a dicotyledon.

The plant may for example be selected from the group consisting of wheat, maize, rapeseed, rice, oat, barley, sugarcane, sunflower, soybean, cotton, potato and tomato.

The plant as defined above is preferably an agronomic plant.

By the term “agronomic plant”, it is herein meant a plant suitable for production on a large scale, in particular for human and animal food or for industrial purposes, such as biofuel.

### Gene of Interest

The gene of interest is preferably a gene whose expression in a plant results in at least one phenotype of interest.

A phenotype of interest is for example:
an herbicide tolerance,
a resistance, such as an insect resistance, a fungal resistance, a bacterial resistance, a stress resistance (for example as a water stress resistance),
a reproductive capability, for example fertility,
an improved performance in the fields, such as an improved yield, a tolerance to abiotic stress or tolerance to biotic stress,
an improved performance in an industrial process, for example an improved biofuel production or
an improved nutritional value, for example an increased oil content.

Herbicide tolerance may for example be tolerance to PPO (protoporphyrinogen oxidase) inhibitor herbicide (see for example WO201522636, WO201592706) or tolerance to EPSPS (5-enolpyruvylshikimate-3-phosphate synthase) inhibitors, such as resistance to glyphosate-based herbicides.

The gene of interest may for example be selected from the group consisting of:
an herbicide tolerance gene,
an insect resistance gene, a fungal resistance gene, a bacterial resistance gene, a stress resistance gene,
a gene involved in a reproductive capability (for example a gene involved in fertility restoration, such as a CMS (cytoplasmic male sterility) restorer gene),
a gene involved in performance in the fields (for example, a gene allowing an improved yield, a tolerance to abiotic stress or a tolerance to biotic stress),
a gene involved in performance in an industrial process and
a gene involved in nutritional value of a plant (for example, a gene allowing an increased oil content).

The herbicide tolerance gene may for example be selected from the group consisting of:
a BAR gene, for example the BAR gene of sequence SEQ ID NO: 1,
a gene encoding a wheat acetyl-CoA Carboxylase (ACCase) (for example ACCase chrA SEQ ID NO: 29 encoding SEQ ID NO: 30, ACCase chrB SEQ ID NO: 31 encoding SEQ ID NO: 32 or ACCase chrD SEQ ID NO: 33 encoding SEQ ID NO: 34) comprising the CoAXium mutation Ala2004Val (U.S. Pat. No. 9,578,880_B2) or other mutations from EP2473022_B1,
a gene encoding a wheat Acetolactate Synthase (ALS) mutated individually in an amino-acid (numbering according to the reference Arabidopsis ALS protein encoded by AT3G48560) A122, P197, A205, D376, W574 or S653, or any of the 4 mutations P197, A205, D376 or W574 in combination between them or with A122 or S653, or the combination of mutations D376 and W574, or W574 and S653. A gene encoding wild-type wheat ALS comprises or consist of, for example, ALS chr6 genome A of sequence SEQ ID NO: 35 encoding SEQ ID NO: 36, ALS chr6 genome B of sequence SEQ ID NO: 37 encoding SEQ ID NO: 38, or ALS chr6 genome D of sequence SEQ ID NO: 39 encoding SEQ ID NO: 40. Preferred genes encode a mutated wheat ALS that comprises or consists of the polypeptide sequence of SEQ ID NO: 79 (ALS chr6D with amino-acid substitutions D350E and W548L); SEQ ID NO: 80 (ALS chr6D with amino-acid substitutions W548L and S627N), SEQ ID NO: 81 (ALS chr6A with amino-acid substitutions D350E and W548L), SEQ ID NO: 82 (ALS chr6A with amino-acid substitutions W548L and S627N), SEQ ID NO: 83 (ALS chr6B with amino-acid substitutions D350E and W548L), or SEQ ID NO: 84 (ALS chr6B with amino-acid substitutions W548L and S627N); or
a gene encoding a cytochrome P450 involved in herbicide detoxification. Amongst those cytochrome P450, one can name
   the Lolium rigidum CYP81A10v7 described by Han et al. (2021) (SEQ ID NO: 71 encoding SEQ ID NO: 72),
   The maize CYP81A2 (SEQ ID NO: 73 encoding SEQ ID NO: 74) or ZmCYP81A9 (SEQ ID NO: 75 encoding SEQ ID NO: 76) sequences described by Brazier-Hicks et al. (2022)
   a wheat gene (TraesCS5A02G398000) of sequence SEQ ID NO: 41 encoding SEQ ID NO: 42, which is orthologous to the Lolium rigidum CYP81A10v7 sequence,
   a wheat CYP71 gene (SEQ ID NO: 77 encoding SEQ ID NO: 78) present under the Su1 QTL for chlorotoluron herbicide tolerance,
   or any wheat cytochrome P450 gene (and orthologous genes from the poaceae clade) involved in herbicide detoxification (Barret 1995; Dimaano and Iwakami, 2020).
a gene encoding a glutamine synthetase GS1 mutated in amino-acid 59 (numbering according to the Eleusine indica GS1-1 protein with GenBank accession number on the NCBI server UJO02307.1, entry from Jan. 29, 2022) as described by Zhang et al. (2022) and/or amino-acid 296 as described in WO2021000870), or a glutamine synthetase GS2 mutated in amino-acid 171 (D171N) (numbering according to the Lolium rigidum GS2 protein with GenBank accession number on the NCBI server QEG99483.1, entry from Aug. 28, 2019) as described by Avila-Garcia et al. (2012).

The fertility restorer gene may for example be selected from the group consisting of a RFL29a gene (for example of sequence SEQ ID NO: 17), a RFL79 gene (for example of sequence SEQ ID NO: 19) and Rfo gene (for example of sequence SEQ ID NO: 55).

The insect resistance gene may for example be Cry1Ac gene (for example of sequence SEQ ID NO: 50) or a Cry1Ac gene that has an N-terminal chloroplast targeting signal from Rubisco Activase (RCA), also referred to as RCA-Cry1Ac (for example of sequence SEQ ID NO: 52).

In one preferred embodiment, the gene of interest is for example selected from the group consisting of the BAR gene, ALS gene, GS, cyt P450 gene, RFL29a gene, RFL79 gene, Rfo gene and Cry1Ac gene.

One or at least two genes of interest (for example two, three or at least four genes of interest) may be integrated in the plant in 5′ or in the 3′ of a polyubiquitin gene.

When at least two genes of interest are integrated, these genes may be identical or different. They are preferably different.

By the expression «gene X», it is herein meant (i) gene X, (ii) the cDNA corresponding to gene X, (iii) a nucleic acid encoding the protein encoded by gene X or (iv) a nucleic acid encoding a protein having at least 90% identity with the protein encoded by gene X, preferably at least 95% identity, more preferably at least 98% identity, provided that both proteins share the same or a similar biological activity (in particular resulting in the same phenotype of interest).

### Vector

The present invention particularly relates to a vector suitable for a targeted integration of at least one gene of interest in a plant in 5′ or in 3′ of a polyubiquitin gene.

The vector may be a plasmid.

The vector as defined above comprises a repair DNA, wherein said repair DNA comprises from 5′ to 3′:
a first gRNA target,
a left ubiquitin-like region,
at least one gene of interest,
a right ubiquitin-like region, and
a second gRNA target.

The gene of interest is particularly as defined above in the section “gene of interest”.

The polyubiquitin gene is an endogenous polyubiquitin gene of the plant.

The plant may comprise several polyubiquitin genes.

The polyubiquitin gene is preferably under the control of a strong promoter.

The polyubiquitin gene is for example the wheat Ubi7AL gene of sequence SEQ ID NO: 5, the wheat Ubi7BL gene of sequence SEQ ID NO: 4, wheat Ubi7DL gene of sequence SEQ ID NO: 3, the maize gene of sequence SEQ ID NO: 43, the maize gene of sequence SEQ ID NO: 44, the maize gene of sequence SEQ ID NO: 45, the maize gene of sequence SEQ ID NO: 46, the B. napus gene of sequence SEQ ID NO: 57, the B. napus gene of sequence SEQ ID NO: 58 or the B. napus gene of sequence SEQ ID NO: 59.

The polyubiquitin gene comprises tandem repeats, hereafter referred to as “repeats” or “Ubi repeats”, each repeat encoding an ubiquitin protein.

By the expression “targeted integration in 3′ of a polyubiquitin gene”, it is herein meant that integration of the at least one gene of interest occurs upstream of the stop codon of the polyubiquitin gene, or at the stop codon of the polyubiquitin gene, such that the polyubiquitin stop codon is replaced by the first codon of the gene of interest.

By the expression “targeted integration in 5′ of a polyubiquitin gene”, it is herein meant that integration of the at least one gene of interest occurs at the start codon or downstream the start codon, preferably directly following the start codon of the polyubiquitin gene and no further than 60 nucleotides from the start codon. When the integration occurs at the start codon, the start codon of the gene of interest replaces the start codon of the polyubiquitin gene. In this embodiment, the repair DNA for example comprises from 5′ to 3′:
a first gRNA target,
a left ubiquitin-like region,
at least one gene of interest,
a site cleavable by a Ubi protease,
the start codon of the first Ubi repeat of the polyubiquitin gene,
a right ubiquitin-like region, and
a second gRNA target.

The exact location of the integration is defined by the left ubiquitin-like region and the right ubiquitin-like region of the repair DNA.

The site of integration of the at least one gene of interest is chosen so that the Ubi gene and the gene(s) of interest are in frame, in order to obtain a single RNA transcript and then a fusion protein.

The site of integration of the at least one gene of interest is chosen so that the inserted gene of interest is flanked by one or two Ubi protease domains, to obtain a cleavage of the fusion protein and release the encoded protein(s) of interest.

An Ubi protease domain is a site cleavable by an Ubi protease.

The vector may also comprise at least one site cleavable by an Ubi protease, to obtain a cleavage of the fusion protein and the release of the encoded protein(s) of interest.

The vector as defined above is preferably suitable for a targeted integration of at least one gene of interest in a plant in 5′ or in 3′ of a polyubiquitin gene, wherein the integrated gene is flanked by one or two sequences encoding a sequence cleavable by an Ubi protease, in particular to allow releasing of the encoded protein(s) of interest from the Ubi protein(s).

The vector as defined above preferably comprises a repair DNA, wherein said repair DNA comprises from 5′ to 3′:
a first gRNA target,
a left ubiquitin-like region,
optionally, a site cleavable by a Ubi protease, in particular for an integration in 3′ of the polyubiquitin gene,
at least one gene of interest,
optionally, a site cleavable by a Ubi protease, in particular for an integration in 5′ of the polyubiquitin gene,
a right ubiquitin-like region, and
a second gRNA target.

The repair DNA in the vector as defined above preferably comprises a site cleavable by an Ubi protease, wherein said site is in 5′ of the gene of interest for an integration in 3′ of the polyubiquitin gene or is in 3′ of the gene of interest for an integration in 3′ of the polyubiquitin gene.

The site cleavable by an Ubi protease may be provided in the form of a region of the polyubiquitin gene comprising a site recognized by an Ubi protease (for example a region encoding for at least the last 6, 8, 10, 12 or 14 amino acids of the C-terminus of a protein encoded by any repeat of the polyubiquitin gene except the last repeat).

As defined herein, an ubiquitin-like region preferably comprises:
a sequence homologous to a sequence comprising the end of the coding region of the polyubiquitin gene and at least a portion of the 3′UTR region of the polyubiquitin gene, for a targeted insertion in 3′ of the polyubiquitin gene, or
a sequence homologous to a sequence comprising at least a portion of the 5′UTR region of the polyubiquitin and the start of the coding region of the polyubiquitin gene, for a targeted insertion in 5′ of the polyubiquitin gene.

The ubiquitin-like region as defined above comprises a left ubiquitin-like region in 5′ and a right ubiquitin-like region in 3′. Such an ubiquitin-like region may thus comprise, between the left ubiquitin-like region and the right ubiquitin-like region, a sequence homologous to a sequence of the polyubiquitin gene, which is lost upon the targeted integration of the at least one gene of interest. Alternatively, the ubiquitin-like region may consist of a left ubiquitin-like region in 5′ and a right ubiquitin-like region in 3′.

By the expression “a sequence homologous to sequence X”, it is particularly meant that said sequence has at least 85% identity with sequence X, preferably at least 90%, more preferably at least 95%, more preferably at least 96%, at least 97%, at least 98% or at least 99% identity with sequence X.

In a preferred embodiment, a sequence homologous to X is identical to sequence X.

The expression “a region Y is homologous to a region X” herein means that the sequence of region Y is homologous to the sequence of region X.

The present invention preferably relates to a vector as defined above,
wherein said left ubiquitin-like region and said right ubiquitin-like region are homologous to a region in 5′ and in 3′, respectively, of a sequence comprising the end of the coding region of the polyubiquitin gene and at least a portion of the 3′UTR region of the polyubiquitin gene, for a targeted insertion in 3′ of the polyubiquitin gene, and
wherein said left ubiquitin-like region and said right ubiquitin-like region are homologous to a region in 5′ and in 3′, respectively, of a sequence comprising at least a portion of the 5′UTR region of the polyubiquitin and the start of the coding region of the polyubiquitin gene for a targeted insertion in 5′ of the polyubiquitin gene.

When the plant comprises more than one polyubiquitin gene, the ubiquitin-like domain preferably comprises a sequence homologous to a sequence present in only one of these polyubiquitin genes, preferably a sequence homologous to 5′ UTR or 3′ UTR. The 5′UTR and 3′UTR indeed comprise variations between the different polyubiquitin genes in a genome that allow the targeting of a specific polyubiquitin gene.

The ubiquitin-like region preferably comprises at least 50 nucleotides, preferably at least 100 nucleotides, preferably at least 400 nucleotides, preferably at least 700 nucleotides, preferably at least 900 nucleotides, preferably at least 1200 nucleotides, more preferably at least 1400 nucleotides and/or at most 1900 nucleotides, preferably at most 1700 nucleotides, more preferably at most 1900 nucleotides.

The left and/or right ubiquitin-like region preferably comprises at least 50 nucleotides, preferably at least 100 nucleotides, preferably at least 200 nucleotides, more preferably at least 300 nucleotides, more preferably at least 400 nucleotides, more preferably at least 500 nucleotides, more preferably at least 600 nucleotides and/or at most 1900 nucleotides, preferably at most 1700 nucleotides, more preferably at most 1500 nucleotides, more preferably at most 1300 nucleotides, more preferably at most 1100 nucleotides, more preferably at most 900 nucleotides.

For an insertion in 3′ of the polyubiquitin gene, the left ubiquitin-like region may comprise the last repeat of the polyubiquitin gene, preferably the two last repeats of the polyubiquitin gene, more preferably the three last repeats of the polyubiquitin gene. For example, the left ubiquitin-like region may comprise repeats 3 to 5 of the polyubiquitin gene for an insertion in 3′ of the polyubiquitin gene.

For an insertion in 5′ of the polyubiquitin gene, the left ubiquitin-like region may comprise a portion of the 5′ UTR or the 5′ UTR of the polyubiquitin gene.

For an insertion in 3′ of the polyubiquitin gene, the right ubiquitin-like region may comprise a portion of the 3′UTR or the 3′UTR of the polyubiquitin gene. For example, the right ubiquitin-like region may comprise the terminator of the polyubiquitin gene plus adjacent intergenomic regions for an insertion in 3′ of the polyubiquitin gene.

For an insertion in 5′ of the polyubiquitin gene, the right ubiquitin-like region may for example comprise repeats 1 to 3 of the polyubiquitin gene.

A left ubiquitin-like region, in particular suitable for a 3′ insertion, may for example comprise or consist of sequence SEQ ID NO: 66, which is homologous to repeats 3 to 5 of the polyubiquitin gene Ubi7DL.

A right ubiquitin-like region, in particular suitable for a 5′ insertion, may for example comprise or consist of sequence SEQ ID NO: 67, which is homologous to repeats 1 to 3 of the polyubiquitin gene Ubi7DL.

A right ubiquitin-like region, in particular suitable for a 3′ insertion, may for example comprise or consist of sequence SEQ ID NO: 68, which is homologous to a region of the 3′UTR region of the polyubiquitin gene Ubi7DL.

A left ubiquitin-like region, in particular suitable for a 5′ insertion, may for example comprise or consist of sequence SEQ ID NO: 69, which is homologous to a region of the 5′UTR region of the polyubiquitin gene Ubi7DL.

The first gRNA target and the second gRNA target comprise a sequence complementary to a gRNA.

In one embodiment, the first gRNA target and the second gRNA target may comprise a sequence complementary to the same gRNA.

The gRNA target preferably comprises at least 15 nucleotides, preferably at least 17 nucleotides, more preferably at least 18 nucleotides and/or at most 25 nucleotides, preferably at most 22 nucleotides, more preferably at most 20 nucleotides.

The gRNA target for example consists of 17, 18, 19 or 20 nucleotides.

The first and second gRNA target sequences are preferably identical.

The first and/or second gRNA target may for example comprise or consist of sequence SEQ ID NO: 8.

The repair DNA as defined above preferably does not encode a polypeptide comprising a cleavable sequence other than sequence(s) cleavable by an Ubi protease.

The expressions “sequence cleavable by an Ubi protease” or “site recognized by a Ubi protease” are herein synonymous.

If needed, in particular depending of the site of integration, the repair DNA as defined above may comprise a sequence cleavable by an Ubi protease, so as to be able to separate the protein of interest from the Ubi protein.

When the repair DNA as defined above comprises at least two genes of interest, the repair DNA further comprises at least one sequence encoding one cleavable sequence between each two genes of interest, said cleavable sequence(s) being a sequence cleavable by an Ubi protease.

The proper release of the protein of interest from the polyubiquitin fusion protein is also important for proteins addressed to one or more different cellular compartments in particular to the mitochondria or chloroplast. The person skilled in the art knows that some proteins have to be addressed to one of these compartment, for example: fertility restorers are supposed to be required in mitochondria, or some herbicides as ALS are known to be active in the chloroplast. It is also one of the scope of the invention to provide a method to obtain expression of a protein active in those cellular compartments.

The repair DNA as defined above preferably does not comprise any T2A or 2A sequence, nor any IRES sequence.

The repair DNA as defined above preferably does not comprise any T2A sequence, any 2A sequence or any IRES sequence.

The repair DNA as defined above preferably does not comprise any T2A sequence, any 2A sequence and any IRES sequence.

By “T2A sequence” or “2A sequence”, it is herein meant self-cleaving peptide motifs derived from certain viruses such as the Foot and Mouth Virus.

The vector as defined above preferably does not comprise any T2A sequence.

The vector as defined above preferably does not comprise any 2A sequence.

The vector as defined above preferably does not comprise any IRES sequence.

The vector as defined above preferably does not comprise any T2A, any 2A sequence and any IRES sequence.

In one preferred embodiment, the repair DNA as defined above only comprises gRNA targets at its 5′ and 3′ ends, to avoid cleavage within the repair DNA.

In another embodiment, the repair DNA as defined above does not comprise any gRNA target other than the first and second gRNA targets, in particular to avoid any cleavage once the repair DNA is inserted. For that purpose, mutation(s) may for example be introduced in the sequence of the gene(s) of interest, in the left ubiquitin-like region and/or in the right ubiquitin-like region, as compared to the corresponding wild-type sequences. Preferably, said mutation(s) do not result in a change in the sequence of the protein of interest encoded by the gene of interest in the repair DNA, compared to the sequence of the wild-type protein.

The vector comprising the repair DNA as defined above may further comprise:
at least one CRISPR (Clustered Regularly Interspaced Short Palindromic Repeats)-Cas endonuclease expression cassette and/or
at least one gRNA expression cassette.

Alternatively, (i) the at least one CRISPR-Cas endonuclease expression cassette and/or (ii) the at least one gRNA expression cassette may be provided in the form of one or several separate vector(s) comprising said cassettes.

The CRISPR-Cas endonuclease expression cassette comprises a nucleic acid encoding a Cas endonuclease under the control of a promoter.

The Cas endonuclease is an enzyme which uses a gRNA as a guide to recognize and performs a double-stranded break at a specific position in a DNA sequence. The Cas endonuclease generally requires the presence of a Protospacer Adjacent Motif (PAM) sequence in the vicinity of the specific targeted position. The PAM sequence can differ depending on the Cas endonuclease.

When the used Cas endonuclease requires a PAM sequence, the first gRNA target and/or the second gRNA target further comprises a PAM sequence.

The Cas endonuclease may be selected from the group consisting of Cas9, Cas12a, Cas12b, C2c1 and C2c2.

The Cas endonuclease is preferably Cas9 (CRISPR-associated protein 9) endonuclease. Cas9 endonuclease may for example comprise or consist of sequence SEQ ID NO: 13.

The promoter of the CRISPR-Cas endonuclease expression cassette may be a constitutive promoter selected from the group consisting of an ZmUbi promoter, the 35S promoter or the 19S promoter (Kay et al., 1987), the rice actin promoter (McElroy et al., 1990), the pCRV promoter (Depigny-This et al., 1992), the CsVMV promoter (Verdaguer et al., 1998) and the ubiquitin promoter from rice or sugarcane. The promoter of the CRISPR-Cas endonuclease expression cassette is preferably an ZmUbi promoter.

The CRISPR-Cas endonuclease expression cassette preferably comprises a terminator, for example SbHSP.

The gRNA expression cassette comprises a nucleic acid encoding a gRNA under the control of a promoter.

The gRNA comprises:
a region complementary to the first and/or second gRNA target sequence and/or to a region in 5′ or 3′ of the polyubiquitin gene, and
a scaffold region that allows binding to the CRISPR-Cas endonuclease encoded by the CRISPR-Cas endonuclease expression cassette.

The promoter of the gRNA expression cassette may for example be selected from the group consisting of an RNA polymerase III promoter (for example TaU6 promoter, ZmU6 promoter or ZmU3), or an RNA polymerase II promoter such as a constitutive promoter (for example ZmUbi or TaUbi). The promoter of the gRNA expression cassette is preferably TaU6 promoter.

The gRNA produced by the gRNA expression cassette is able to:
recognize the first and/or second gRNA target of the repair DNA, so as to liberate the repair DNA from the vector through the action of the corresponding CRISPR-CAS endonuclease, and/or
recognize a region in 3′ or 5′ of the polyubiquitin gene, so as to introduce a double stranded break in 3′ or 5′, respectively, of the polyubiquitin gene through the action of the corresponding CRISPR-CAS endonuclease.

In one embodiment, the vector comprising a repair DNA as defined above comprises a single gRNA expression cassette.

When a single gRNA expression cassette is used, the gRNA is able to introduce a double stranded break both in 3′ or 5′ of the polyubiquitin gene and to liberate the repair DNA. The sequence complementary to the gRNA is in this case identical in the first and second gRNA targets of the repair DNA and in the 3′ or 5′ of the polyubiquitin gene.

When two gRNA expression cassettes are used, the first gRNA transcribed from the first gRNA expression cassette is for example able to introduce a double stranded break in 3′ or 5′ of the polyubiquitin gene and the second gRNA transcribed from the second gRNA expression cassette is for example able to liberate the repair DNA. The sequence complementary to the second gRNA is in this case identical in the first and second gRNA targets of the repair DNA.

When three gRNA expression cassettes are used, the first gRNA transcribed from the first gRNA expression cassette is for example able to introduce a double stranded break in 3′ or 5′ of the polyubiquitin gene, the second gRNA transcribed from the second gRNA expression cassette is for example able to liberate the 5′ end of the repair DNA and the third gRNA transcribed from the third gRNA expression cassette is for example able to liberate the 3′ end of the repair DNA.

When four gRNA expression cassettes are used,
the first gRNA transcribed from the first gRNA expression cassette is for example able to introduce a double stranded break in 3′ or 5′ of the polyubiquitin gene,
the second gRNA transcribed from the second gRNA expression cassette is for example able to introduce a double stranded break in 3′ or 5′ of the polyubiquitin gene, downstream of the double stranded break introduced by the first gRNA,
the third gRNA transcribed from the third gRNA expression cassette is for example able to liberate the 5′ end of the repair DNA and
the fourth gRNA transcribed from the fourth gRNA expression cassette is for example able to liberate the 3′ end of the repair DNA.

The vector comprising the repair DNA as defined above may further comprise a selection marker.

Any suitable selection marker well-known by the skilled person may be used. The selection marker may for example be NptII gene or bar gene.

NptII (neomycin phosphotransferase) inactivates aminoglycoside antibiotics, including kanamycin and neomycin.

The selection marker is preferably provided in the form of a selection marker expression cassette comprising said selection marker.

When at least two genes of interest are to be integrated in a plant genome, in particular in 5′ or 3′ of the polyubiquitin gene, they may be provided in the same repair DNA or in different repair DNAs.

When at least two genes of interest are to be integrated in a plant genome, in particular in 5′ or 3′ of the polyubiquitin gene, they may be provided in different repair DNAs either in the same vector or in different vectors.

For example, a first vector as defined above comprises a repair DNA comprising the first gene of interest and a second vector as defined above comprises a repair DNA comprising the second gene of interest

In a preferred embodiment, the at least two genes of interest are provided in the same vector, more preferably in the same repair DNA. In such case, it is preferred that the repair DNA comprises a site recognized by a Ubi protease between each two genes of interest. This allows processing between the first and the second genes of interest, between the second and third genes of interest (if a third gene of interest is present), etc.

The site recognized by an Ubi protease is for example a 3′ ubiquitin tail coding for at least the last 6 amino acids, preferably at least the last 8 amino acids, preferably at least the last 10 amino acids, preferably at least the last 12 amino acids, or still preferably at least the last 14 amino acids of the C-term of a protein encoded by any of the Ubi repeats, except the last Ubi repeat. The site recognized by an Ubi protease may for example comprise or consist of the last 14 amino acids of the C-term of a protein encoded by one repeat of Ubi7DL (except the last Ubi repeat). A site recognized by an Ubi protease may for example comprise or consist of sequence SEQ ID NO: 70.

The vector as defined above suitable for targeted integration of at least two genes of interest may for example comprise a repair DNA, wherein said repair DNA comprises from 5′ to 3′:
a first gRNA target, in particular as defined above,
a left ubiquitin-like domain, in particular as defined above,
optionally, a region of the polyubiquitin gene comprising a site recognized by an Ubi protease (for example a region encoding for at least the last 6, 8, 10, 12 or 14 amino acids of the C-terminus of a protein encoded by any repeat of the polyubiquitin gene except the last repeat), in particular for an integration in 3′ of the polyubiquitin gene,
the first gene of interest,
a region of the polyubiquitin gene comprising a site recognized by an Ubi protease (for example a region encoding for at least the last 6, 8, 10, 12 or 14 amino acids of the C-terminus of a protein encoded by any repeat of the polyubiquitin gene except the last repeat),
the second gene of interest,
optionally, a region of the polyubiquitin gene comprising a site recognized by an Ubi protease (for example a region encoding for at least the last 6, 8, 10, 12 or 14 amino acids of the C-terminus of a protein encoded by any repeat of the polyubiquitin gene except the last repeat), in particular for an integration in 3′ of the polyubiquitin gene,
the right ubiquitin-like domain, in particular as defined above, and
a second gRNA target, in particular as defined above.

If more than two genes of interest are integrated, a region of the polyubiquitin gene comprising a site recognized by a Ubi protease is introduced between each two genes of interest.

The Ubi protease is preferably the endogenous Ubi protease expressed by the plant cell.

When the fusion protein comprising the at least one gene of interest is expressed in a plant cell, the Ubi protease expressed in said cell can thus cut said fusion protein, thereby releasing the or each protein encoded by the gene(s) of interest.

The targeted insertion of at least one gene of interest in 5′ or in 3′ of the polyubiquitin gene is thus achieved by using at least one repair DNA, at least one CRISPR-Cas endonuclease expression cassette and at least one gRNA expression cassette, which elements are provided either in the same vector or in separate vectors. Beside said gRNA expression cassette(s) and said CRISPR-Cas endonuclease expression cassette(s) need to provide gRNA(s) and Cas-endonuclease(s) able to:
liberate the repair DNA from the vector, and
introduce a double stranded break in 5′ or in 3′ of the polyubiquitin gene.

The invention also relates to an inserted fragment in the wheat Ubi sequence under the control of an Ubi promoter that allows strong expression of a GOI. A vector comprising an Ubi promoter, such as the Ubi7DL promoter, or either the Ubi7AL or Ubi7BL promoter, with ubiquitin-like regions as described above can drive strong expression of a GOI. Vectors such as the one described in example 4bis make also part of the invention. In particular said vector comprises from 5′ to 3′ a wheat Ubi promoter, a wheat Ubi CDS, at least one gene of interest, and a wheat Ubi terminator. Preferably, said Ubi promoter, Ubi CDS and Ubi terminator are from a same wheat Ubi gene, such as wheat Ubi7AL gene, wheat Ubi7DL gene, or wheat Ubi7BL gene. According to some embodiments, the at least one gene of interest encodes a mutated wheat ALS1 gene that confers herbicide resistance.

The present invention also relates to a plant cell or plant tissue, which is transformed with (i) at least said vector and (ii) optionally, at least one CRISPR-Cas endonuclease expression cassette as defined and/or at least one gRNA expression cassette as defined above. The two or three expression cassette may be on the same vector or different vectors.

One of GOI tested by the inventor is a mutated ALS1 gene. They showed that selection of 2 mutations in ALS1 gene, that result in either mutations at amino-acid D376 and W574 (SEQ ID NO: 79, SEQ ID NO: 81 or SEQ ID NO: 83) or W574 and S653 (SEQ ID NO: 80 or SEQ ID NO: 82 or SEQ ID NO: 84) of the encoded polypeptide (as defined by reference to Arabidopsis protein positions) can lead to strong herbicide resistance.

Accordingly, the invention further relates to an isolated nucleic acid that encodes a wheat ALS1 mutated polypeptide sequence at amino-acids D376 and W574, or at amino-acids W574 and S653, by reference to Arabidopsis protein positions, or at amino-acids D350 and W548, or W548 and S627 by reference to wheat chromosome 6 genome A, B or D. Preferably, the isolated nucleic acid encodes a mutated wheat ALS1 polypeptide comprising the amino acid substitutions D350E and W548L, or amino acid substitutions 548L and S627N in either of SEQ ID NO: 36, SEQ ID NO: 38, or SEQ ID NO: 40. Preferably, the isolated nucleic acid encodes a mutated wheat ALS1 polypeptide that does not comprise additional mutations. According to come embodiments, the isolated nucleic acid encodes a mutated wheat ALS1 polypeptide comprising or consisting of SEQ ID NO: 79, SEQ ID NO: 80, SEQ ID NO: 81, SEQ ID NO: 82, SEQ ID NO: 83, or SEQ ID NO: 84.

The invention further relates to a vector comprising a nucleic acid encoding a mutated wheat ALS1 polypeptide, as described above.

Plants transformed with nucleic acid encoding a mutated wheat ALS1 polypeptide, or with a vector comprising said nucleic acid encoding a mutated wheat ALS1 polypeptide also make part of the invention.

### Plant Cell, Plant Tissue or Plant Expressing at Least One Gene of Interest

The present invention also relates to a plant cell or plant tissue, which is transformed with (i) at least one vector as defined above in the section “Vector” and (ii) optionally, at least one CRISPR-Cas endonuclease expression cassette as defined above in the section “Vector” and/or at least one gRNA expression cassette as defined above in the section “Vector”.

The present invention also relates to a plant cell or plant tissue comprising at least one gene of interest integrated in 5′ or in 3′ of the polyubiquitin gene, in particular obtained by transformation with (i) at least one vector as defined above in the section “Vector” and (ii) optionally, at least one CRISPR-Cas endonuclease expression cassette as defined above in the section “Vector” and/or at least one gRNA expression cassette as defined above in the section “Vector”.

The plant cell or plant tissue as defined above may be obtained by the method for targeted insertion of a gene of interest in 5′ or in 3′ of a polyubiquitin gene in a plant genome as defined below.

The plant is particularly as defined above in the section “Plant”.

The plant cell may be a protoplast.

The plant tissue may be an apical meristem, cotyledon, embryo, pollen and/or microspores.

The present invention also relates to a plant comprising at least one gene of interest, in particular integrated in 5′ or 3′ of the polyubiquitin gene, in particular obtained by transformation with the vector as defined above in the section “Vector”, preferably a plant obtained from a plant cell or plant tissue as defined above.

The present invention also relates to a progeny plant of as defined above, wherein said progeny plant comprises at least one gene of interest inserted in 5′ or in 3′ of the polyubiquitin gene.

The plant, progeny plant, plant cell and plant tissue as defined above express at least one protein of interest encoded by at least one gene of interest, in the form of a fusion protein comprising ubiquitin protein(s) and the protein(s) of interest, said fusion protein being then cleaved by endogenous ubi protease, thereby releasing said protein(s) of interest.

### Method for Targeted Insertion of a Gene of Interest

The present invention particularly relates to a method for targeted insertion of a gene of interest in 5′ or in 3′ of a polyubiquitin gene in a plant genome.

The method as defined above particularly comprises:
a) the transformation of a plant cell or plant tissue, in particular as defined above in the section “Plant cell, plant tissue or plant expressing at least one gene of interest”, with at least one vector comprising a repair DNA, as defined above in the section “Vector”, to obtain a transformed plant cell or a transformed plant tissue and
b) the regeneration of a plant from the transformed plant cell or transformed plant tissue.

Step a) comprises the transformation of a plant cell or plant tissue, in particular as defined above in the section “Plant cell, plant tissue or plant expressing a gene of interest” with at least one vector comprising a repair DNA, optionally at least one CRISPR-Cas endonuclease expression cassette and optionally at least one gRNA expression cassette.

If the vector comprising the repair DNA does not comprise at least one CRISPR-Cas endonuclease expression cassette and/or does not comprise at least one gRNA expression cassette, the plant cell or plant tissue is transformed with separate vector(s) comprising the missing expression cassette(s), i.e. at least one CRISPR-Cas endonuclease expression cassette and/or at least one gRNA expression cassette.

If the vector comprising the repair DNA does not comprise at least one CRISPR-Cas endonuclease expression cassette, nor at least one gRNA expression cassette, the plant cell or plant tissue is preferably transformed with a second vector comprising at least one CRISPR-Cas endonuclease expression cassette and at least one gRNA expression cassette. Alternatively, the CRISPR-Cas endonuclease expression cassette and the gRNA expression cassette may be provided in separate vectors.

When more than one vector is used in step a), the plant cell or plant tissue is preferably transformed with the different vectors at the same time.

Any technique suitable for plant cell or plant tissue transformation may be used, such as biolistic particle delivery, PEG transformation, electroporation or Agrobacterium transgene delivery.

For Agrobacterium transgene delivery, the vector(s) is/are first transferred into Agrobacterium, to obtain a transformed Agrobacterium and the plant cell or plant tissue is then transformed with said transformed Agrobacterium. The Agrobacterium is preferably Agrobacterium tumefaciens.

Step a) particularly results in:
(a1) the expression of the Cas endonuclease(s) from the CRISPR-Cas endonuclease expression cassette(s) and the production of gRNA(s) from the gRNA expression cassette(s), thereby producing a double strand break in 3′ or 5′ of the polyubiquitin gene in the plant genome and two double strand breaks in the vector, thereby liberating the repair DNA, and
(a2) the homologous recombination between the repair DNA and the plant genome in 3′ or 5′ of the polyubiquitin gene.

The (a2) step can also occur during or after the regeneration of the plant (step (b)).

Step b) comprises regeneration of a plant.

Regeneration of a plant from a plant cell or plant tissue is well known by the skilled person.

In particular, the plant cell or plant tissue may be placed in a culture medium suitable for plant growth.

The regeneration of a plant from a plant cell or a plant tissue may comprise:
growing said plant cell or plant tissue to obtain a callus, and
regeneration of shoots from the callus.

The growth of the plant cell into a callus and the regeneration of shoots are carried out in any suitable culture medium comprising plant growth regulators.

The regeneration of a plant from a plant tissue may comprise regeneration of shoots.

The regeneration of shoots from the plant tissue is carried out in any suitable culture medium comprising plant growth regulators.

### Method for Expressing a Protein of Interest in a Plant

The present invention particularly relates to a method for expressing at least one protein of interest in a plant, wherein said method comprises the steps of the method for targeted insertion of at least one gene of interest in 5′ or 3′ of a polyubiquitin gene in a plant genome as defined above, wherein said gene of interest codes for said protein of interest.

The method as defined above may further comprise a subsequent step of detecting the protein of interest.

Detection of a protein of interest may be performed according to any method well-known by the skilled person, such as western-blot or an immunoassay.

### Use of a Vector for Expressing a Gene of Interest in a Plant

The present invention also relates to the use of a vector comprising a repair DNA as defined above in the section “Vector”, for expressing at least one gene of interest in a plant, plant cell or plant tissue.

The plant, plant cell and plant tissue are particularly as defined above.

The use as defined above particularly allows expressing said at least one gene of interest under the endogenous promoter of the polyubiquitin gene.

The present invention also relates to the use of a vector comprising a repair DNA as defined above in the section “Vector” for the targeted integration of at least one gene of interest, in particular in 5′ or 3′ of a polyubiquitin gene.

### Method of Identifying a Plant Having a Gene of Interest Inserted in 5′ or 3′ of a Polyubiquitin Gene

The present invention also relates to a method of identifying a plant comprising at least one gene of interest inserted in 5′ or 3′ of a polyubiquitin gene, wherein said method comprises:
extracting the DNA, RNA or proteins of a plant,
detecting the presence of a DNA comprising at least one gene of interest inserted in 5′ or 3′ of a polyubiquitin gene and/or the presence of an RNA transcript from said DNA, and
optionally, detecting the presence of a protein encoded by said at least one gene of interest.

Detecting the presence of a DNA comprising at least one gene of interest inserted in 5′ or 3′ of a polyubiquitin gene may for example comprise detecting the presence of the repair DNA as defined above in the section “Vector” or of a fragment thereof.

Said fragment thereof may for example comprise:
the left ubiquitin-like domain and at least the 5′ part of the gene of interest (or of the first gene of interest), or
at least the 3′ part of the gene of interest (or of the last gene of interest) and the right ubiquitin-like domain.

In one embodiment, detecting the presence of a DNA comprising at least one gene of interest inserted in 5′ or 3′ of a polyubiquitin gene is performed using at least one pair of PCR primers, in particular able to amplify a region comprising a portion of the endogenous plant genome and at least a portion of the gene of interest, for example,
a pair of primers comprising one primer that recognizes a region upstream of the left ubiquitin-like region in the genome and which is not present in the repair DNA and another primer that recognizes at least one portion of the gene of interest, and/or
a pair of primers comprising a primer that recognizes at least one portion of the gene of interest and a primer that recognizes a region downstream of the right ubiquitin-like region in the genome and which is not present in the repair DNA.

Detecting the presence of a protein encoded by said at least one gene of interest allows confirming that the protein is expressed and/or assessing its level of expression.

Detecting the presence of a protein encoded by said at least one gene of interest may be performed as follows:
if the gene of interest encodes a protein that is already present in the wild-type plant, the quantity of protein produced is for example compared to the quantity of protein produced by a control wild-type plant. If the quantity produced by the transformed plant is significantly higher than the quantity produced in a control wild-type plant, the plant is identified as a plant expressing the protein encoded by the gene of interest,
if the gene of interest encodes a protein absent from the wild-type plant, the detection of the presence of the protein in the transformed plant allows the identification of a plant expressing the protein encoded by the gene of interest.

If the gene of interest in the repair DNA is also a selective marker, for example an herbicide gene like the Bar gene, the expression of the protein can be detected in a selective medium.

The invention will be further illustrated in the following figures and examples.

### BRIEF DESCRIPTION OF THE SEQUENCES

| Sequence | | Origin (organisme or | Name of the |
|---|---|---|---|
| number | Type | artificial) | sequence |
| SEQ ID NO: 1 | Nucleic | Streptomyces | phosphinothricin |
| | | hygroscopicus strain A10 | acetyl transferase |
| | | | gene (bar gene) |
| SEQ ID NO: 2 | Protein | Streptomyces | phosphinothricin |
| | | hygroscopicus strain A10 | acetyl transferase |
| | | | (bar protein) |
| SEQ ID NO: 3 | Nucleic | Triticum aestivum | TaUbi7DL genomic |
| | | (var. Fielder) | |
| SEQ ID NO: 4 | Nucleic | Triticum aestivum | TaUbi7BL genomic |
| | | (var. Fielder) | |
| SEQ ID NO: 5 | Nucleic | Triticum aestivum | TaUbi7AL genomic |
| | | (var. Fielder) | |
| SEQ ID NO: 6 | Nucleic | Triticum aestivum | G1 gRNA spacer |
| | | (var. Fielder) | |
| SEQ ID NO: 7 | Nucleic | Triticum aestivum | G2 gRNA spacer |
| | | (var. Fielder) | |
| SEQ ID NO: 8 | Nucleic | Triticum aestivum | G3 gRNA spacer |
| | | (var. Fielder) | |
| SEQ ID NO: 9 | Nucleic | Triticum aestivum | TaU6 promoter |
| SEQ ID NO: 10 | Nucleic | Artificial | TaU6_G1, G2, G3 |
| | | | mutiplex gRNA |
| SEQ ID NO: 11 | Nucleic | Zea mays | ZmUbiquitin |
| | | | promoter + 5′UTR |
| SEQ ID NO: 12 | Nucleic | Agrobacterium tumefaciens | Nopaline synthase |
| | | | polyadenylation |
| | | | sequence |
| SEQ ID NO: 13 | Nucleic | Artificial | SV40 NLS-SpCas9- |
| | | | Nucleoplasmin NLS, |
| | | | maize optimised |
| SEQ ID NO: 14 | Nucleic | Artificial | Ubi7DL-BAR -Ub7DL |
| | | | donor repair fragment |
| | | | flanked by G3 gRNA |
| | | | target sites |
| SEQ ID NO: 15 | Nucleic | Triticum aestivum | TraesCS1B01G038500 |
| | | (var. Chinese Spring) | (RFL29b) CDS |
| SEQ ID NO: 16 | Protein | Triticum aestivum | TraesCS1B01G038500 |
| | | (var. Chinese Spring) | (RFL29b) |
| SEQ ID NO: 17 | Nucleic | Triticum aestivum | RFL29a CDS |
| | | (var. Spelt) | |
| SEQ ID NO: 18 | Protein | Triticum aestivum | RFL29a |
| | | (var. Spelt) | |
| SEQ ID NO: 19 | Nucleic | Triticum timopheevii | RFL79 CDS |
| SEQ ID NO: 20 | Protein | Triticum timopheevii | RFL79 |
| SEQ ID NO: 21 | Nucleic | Artificial | RH-Ubi7DL-RFL29a |
| | | | CDS |
| SEQ ID NO: 22 | Nucleic | Artificial | RFL29a_RH-Ubi7DL |
| | | | CDS |
| SEQ ID NO: 23 | Nucleic | Artificial | RH-Ubi7DL-RFL79 |
| | | | CDS |
| SEQ ID NO: 24 | Nucleic | Artificial | RFL79_RH-Ubi7DL |
| | | | CDS |
| SEQ ID NO: 25 | Nucleic | Artificial | pTaU6_G3 gRNA |
| SEQ ID NO: 26 | Nucleic | Artificial | Ubi7DL-RFL29a - |
| | | | Ub7DL donor repair |
| | | | fragment flanked by |
| | | | G3 gRNA target sites |
| SEQ ID NO: 27 | Nucleic | Artificial | Ubi7DL-RFL79 - |
| | | | Ub7DL donor repair |
| | | | fragment flanked by |
| | | | G3 gRNA target sites |
| SEQ ID NO: 28 | Nucleic | Artificial | Ubi7DL-RFL29a-Ubi- |
| | | | RFL79 -Ub7DL donor |
| | | | repair fragment |
| | | | flanked by G3 gRNA |
| | | | target sites |
| SEQ ID NO: 29 | Nucleic | Triticum aestivum | ACCase ChrA CDS |
| | | (var. Fielder) | |
| SEQ ID NO: 30 | Protein | Triticum aestivum | ACCase ChrA |
| | | (var. Fielder) | |
| SEQ ID NO: 31 | Nucleic | Triticum aestivum | ACCase ChrB CDS |
| | | (var. Fielder) | |
| SEQ ID NO: 32 | Protein | Triticum aestivum | ACCase ChrB |
| | | (var. Fielder) | |
| SEQ ID NO: 33 | Nucleic | Triticum aestivum | ACCase ChrD CDS |
| | | (var. Fielder) | |
| SEQ ID NO: 34 | Protein | Triticum aestivum | ACCase ChrD |
| | | (var. Fielder) | |
| SEQ ID NO: 35 | Nucleic | Triticum aestivum | ALS ChrA CDS |
| | | (var. Fielder) | |
| SEQ ID NO: 36 | Protein | Triticum aestivum | ALS ChrA |
| | | (var. Fielder) | |
| SEQ ID NO: 37 | Nucleic | Triticum aestivum | ALS ChrB CDS |
| | | (var. Fielder) | |
| SEQ ID NO: 38 | Protein | Triticum aestivum | ALS ChrB |
| | | (var. Fielder) | |
| SEQ ID NO: 39 | Nucleic | Triticum aestivum | ALS ChrD CDS |
| | | (var. Fielder) | |
| SEQ ID NO: 40 | Protein | Triticum aestivum | ALS ChrD |
| | | (var. Fielder) | |
| SEQ ID NO: 41 | Nucleic | Triticum aestivum | P450_Chr5A, |
| | | (var. Chinese Spring) | TraesCS5A02G398000 |
| | | | CDS |
| SEQ ID NO: 42 | Protein | Triticum aestivum | P450_Chr5A, |
| | | (var. Chinese Spring) | TraesCS5A02G398000 |
| SEQ ID NO: 43 | Nucleic | Zea mays. (var B73) | ZmUbiChr4, |
| | | | Zm00001d053838 genomic |
| SEQ ID NO: 44 | Nucleic | Zea mays. (var B73) | ZmUbiChr5, |
| | | | Zm0001d015327 |
| | | | genomic |
| SEQ ID NO: 45 | Nucleic | Zea mays. (var A188) | ZmUbiChr4 genomic |
| SEQ ID NO: 46 | Nucleic | Zea mays. (var A188) | ZmUbiChr5, genomic |
| SEQ ID NO: 47 | Nucleic | Zea mays. (var A188) | gRNA31 spacer |
| SEQ ID NO: 48 | Nucleic | Zea mays. (var A188) | gRNA20 spacer |
| SEQ ID NO: 49 | Nucleic | Zea mays | ZmU6 promoter |
| SEQ ID NO: 50 | Nucleic | Artificial | Cry1Ac codon |
| | | | optimised |
| SEQ ID NO: 51 | Protein | Bacillus thuringiensis | Cry1Ac |
| SEQ ID NO: 52 | Nucleic | Artificial | RCA-Cry1Ac codon |
| | | | optimised |
| SEQ ID NO: 53 | Nucleic | Artificial | ZmUbiChr5-Cry1Ac - |
| | | | ZmUbiChr5 donor |
| | | | repair fragment |
| | | | flanked by gRNA20 |
| | | | target sites |
| SEQ ID NO: 54 | Nucleic | Artificial | ZmUbiChr5-RCA- |
| | | | Cry1Ac -ZmUbiChr5 |
| | | | donor repair fragment |
| | | | flanked by gRNA20 |
| | | | target sites |
| SEQ ID NO: 55 | Nucleic | R. sativus | Rfo genomic |
| SEQ ID NO: 56 | Protein | R. sativus | Rfc |
| SEQ ID NO: 57 | Nucleic | B. napus | BnaA09g19810D |
| | | | genomic |
| SEQ ID NO: 58 | Nucleic | B. napus | BnaC09g21810D |
| | | | genomic |
| SEQ ID NO: 59 | Nucleic | B. napus | BnaA08g30590D |
| | | | genomic |
| SEQ ID NO: 60 | Nucleic | B. napus var. Westar | BnUbiA09 genomic |
| SEQ ID NO: 61 | Nucleic | B. napus var. Westar | gRNA16 spacer |
| SEQ ID NO: 62 | Nucleic | Arabidopsis thaliana | AtU6 promoter |
| SEQ ID NO: 63 | Nucleic | Artificial | BnUbiA09-Rfo - |
| | | | BnUbiA09 donor |
| | | | repair fragment |
| | | | flanked by gRNA16 |
| | | | target sites |
| SEQ ID NO: 64 | Nucleic | Cauliflower Mosaic Virus | 35S CaMV promoter |
| SEQ ID NO: 65 | Nucleic | Artificial | SV40 NLS-Cas9- |
| | | | Nucleoplasmin NLS |
| SEQ ID NO: 66 | Nucleic | Triticum aestivum | left ubiquitin-like |
| | | (var. Fielder) | region homologous to |
| | | | repeats 3 to 5 of the |
| | | | polyubiquitin gene |
| | | | Ubi7DL |
| SEQ ID NO: 67 | Nucleic | Triticum aestivum | right ubiquitin-like |
| | | (var. Fielder) | region homologous to |
| | | | repeats 1 to 3 of the |
| | | | polyubiquitin gene |
| | | | Ubi7DL |
| SEQ ID NO: 68 | Nucleic | Triticum aestivum | right ubiquitin-like |
| | | (var. Fielder) | region homologous to |
| | | | a region of the 3′UTR |
| | | | region of the |
| | | | polyubiquitin gene |
| | | | Ubi7DL |
| SEQ ID NO: 69 | Nucleic | Triticum aestivum | left ubiquitin-like |
| | | (var. Fielder) | region homologous to |
| | | | a region of the 5′UTR |
| | | | region of the |
| | | | polyubiquitin gene |
| | | | Ubi7DL |
| SEQ ID NO: 70 | Protein | Triticum aestivum | Cleavable sequence |
| | | (var. Fielder) | by Ubi protase from |
| | | | one repeat of Ubi7DL |
| SEQ ID NO 71 | Nucleic | Lolium rigidum | P450 CYP81A10v7 |
| SEQ ID NO 72 | Protein | Lolium rigidum | P450 CYP81A10v7 |
| SEQ ID NO 73 | Nucleic | Zea mays | P450 CYP81A2 |
| SEQ ID NO 74 | Protein | Zea mays | P450 CYP81A2 |
| SEQ ID NO 75 | Nucleic | Zea mays | P450 CYP81A9 |
| SEQ ID NO 77 | Protein | Zea mays | P450 CYP81A9 |
| SEQ ID NO77 | Nucleic | Triticum aestivum | Su1, P450 CYP71 |
| | | (var. Fielder) | |
| SEQ ID NO 78 | Protein | Triticum aestivum | Su1, P450 CYP71 |
| | | (var. Fielder) | |
| SEQ ID NO 79 | Protein | Triticum aestivum | ALS1_chr6D mutant |
| | | (var. Fielder) | D350E_W548L |
| SEQ ID NO 80 | Protein | Triticum aestivum | ALS1_chr6D mutant |
| | | (var. Fielder) | W548L_S627N |
| SEQ ID NO 81 | Protein | Triticum aestivum | ALS1_chr6A mutant |
| | | (var. Fielder) | D353E_W551L |
| SEQ ID NO 82 | Protein | Triticum aestivum | ALS1_chr6A mutant |
| | | (var. Fielder) | W551L S630N |
| SEQ ID NO 83 | Protein | Triticum aestivum | ALS1_chr6B mutant |
| | | (var. Fielder) | D350E W548L |
| SEQ ID NO 84 | Protein | Triticum aestivum | ALS1_chr6B mutant |
| | | (var. Fielder) | W548L S627N |
| SEQ ID NO 85 | Nucleic | Artificial | TaUbi7DL_promoter::TaUbi7DL_{—} |
| | | | cds::TaAL |
| | | | S1_chr6D_cds::TaUbi7DL_{—} |
| | | | terminator |

### DESCRIPTION OF THE FIGURES

FIG. 1: Strategy of Gene Targeting (GT) at the Ubi7DL locus. A: landing pad: target locus in wheat; B: T-DNA from Agrobacterium strain (T11561), from pBIOS12163.

FIG. 2: PCR Analysis of BASTA resistant T1 Progeny of T11561 plants. Schematic showing the position of primers used to amplify left (1694 bp) and right (988 bp or 1171 bp) homologous recombination junctions. One primer is within Bar to other is in TaUbi7DL outside of the regions of homology between T11561 and TaUbi7DL (RH Left and RH Right.

FIG. 3: PCR Analysis of BASTA resistant T1 Progeny of T11561 plants. Examples of PCR products from the Left junction amplification (1694 bp), Ubi-bar on the figure. T11561_028 plants have a product of the expected size.

FIG. 4: Constructs designed to insert RFL29a (A) from pBIOS12979 or RFL79 (B) from pBIOS12980 into the TaUbi7DL Landing Pad.

FIG. 5: Construct designed to insert CoAXium mutated (T6123C) ACCase into the TaUbi7DL Landing Pad.

FIG. 6: Construct designed to insert a mutated version of the ALS gene into the TaUbi7DL Landing Pad.

FIG. 7: Construct designed to insert the wheat homologue of the Lolium rigidum P450 CYP81A10v7 gene into the TaUbi7DL Landing Pad.

FIG. 8: Constructs designed to over express ALS native (from pBIOS13536) and mutated gene (from pBIOS13535 and pBIOS13534) in a TaUbi context.

FIG. 9: Herbicide treatment on plants over expressing ALS native or mutated in a TaUbi context.

### EXAMPLES

### Example 1: Expression of an Herbicide Resistance from Wheat PolyUbiquitin: Bar Gene Fusion

As a proof of concept, the BAR gene for herbicide resistance (of sequence SEQ ID NO: 1 encoding SEQ ID NO: 2) was fused to the 3′ of a wheat polyubiquitin gene using homologous recombination (Gene Targeting or GT). The use of Bar allows a positive selection for desired insertion events at the Landing Pad.

A polyubiquitin gene on Chr7DL of Chinese Spring (TraesCS7D01G443100) was found by BLAST analysis to be closest to the gene of the strong Ubiquitin Maize promoter on Chromosome 5 widely used in monocot transgenesis. This gene has homologs on Chr7BL (TraesCS7B01G354200) and Chr7AL (TraesCS7A01G453500). RNAseq data confirmed that all 3 genes are strongly expressed and could thus be used as Landing Pads. The Ubi7DL was chosen as the target for GT and sequenced along with Ubi7AL and Ubi7BL in the wheat variety Fielder to be used for transformation. The 3 Ubi genes (Ubi7DL SEQ ID NO: 3, Ubi7BL SEQ ID NO: 4, Ubi7AL SEQ ID NO: 5) have a good level of homology in the CDS but are divergent in the 3′ UTR region suggesting that a GT repair fragment including the Ubi7DL 3′ UTR as one of the arms of homology used for homologous recombination should allow specific targeting to Ubi7DL.

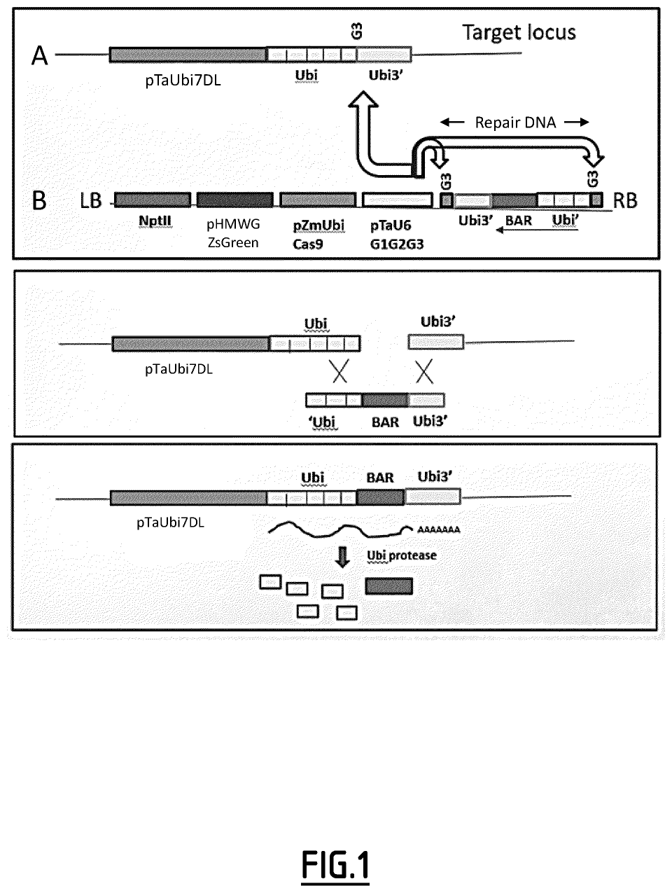

The strategy for GT at Ubi7DL is outlined in FIG. 1 and is based on the method of in planta GT (Fauser et al. 2012). A Cas9 gRNA (G3 SEQ ID NO: 8) that efficiently makes a DNA double strand break (DSB) around the STOP codon of the Ubi7DL gene was first identified by Agrobacterium-mediated transformation into wheat var. Fielder with a binary plasmid containing a TaU6-tRNA multiplex guide (SEQ ID NO: 10) expressing 3 guides G1 (SEQ ID NO: 6), G2 (SEQ ID NO: 7) and G3 (SEQ ID NO: 8) under control of promoter TaU6 (SEQ ID NO: 9), together with ZmUbi (SEQ ID NO: 11)-Cas9 (SEQ ID NO: 13)-nos terminator (SEQ ID NO: 12) and pActin-Bar-nos cassettes. These 3 target sites are around the STOP codon of Ubi7DL. Fielder wheat cultivar was transformed with these Agrobacterium strains essentially as described by WO2000/063398. High-throughput (NGS) sequencing of transformed plantlets showed 59 of 97 (61%) independent transformation events had mutations at the target site and all were generated with the G3 gRNA.

WT Fielder was then transformed with an Agrobacterium strain (T11561) with a binary plasmid pBIOS12163 with a T-DNA that contains the repair DNA (SEQ ID NO: 14 BAR gene flanked by Left (680 bp) and Right homology regions (740 bp) to the Ubi7DL target) flanked by G3 sites. The G3 sites contain 6 bp upstream and 6 bp downstream of Ta7DL sequences flanking the G3 target to help maintain the context of the G3 target. This T-DNA expresses Cas9 (SEQ ID NO: 13) from the constitutive ZmUbi promoter (SEQ ID NO: 11), produces the G3 gRNA from the ZmU6 promoter (SEQ ID NO: 49) and also has NptII under the control of a VirSc4 promoter allowing transient or stable selection of transformants. Expression of Cas9 and the G3 gRNA both produce a DSB at the Ubi7DL target and also liberate the repair DNA from the T-DNA making the repair DNA available for GT at the Ubi7DL target.

Wheat plants stability transformed with T11561 were generated via selection on Kanamycin. In these transformants GT may occur throughout plant growth (provided that the G3 gRNA target sites are not mutated and that the repair DNA is still present). Direct selection on BASTA was also performed on TO plants resistant plants were obtained but molecular analysis shows no GT (table 1).

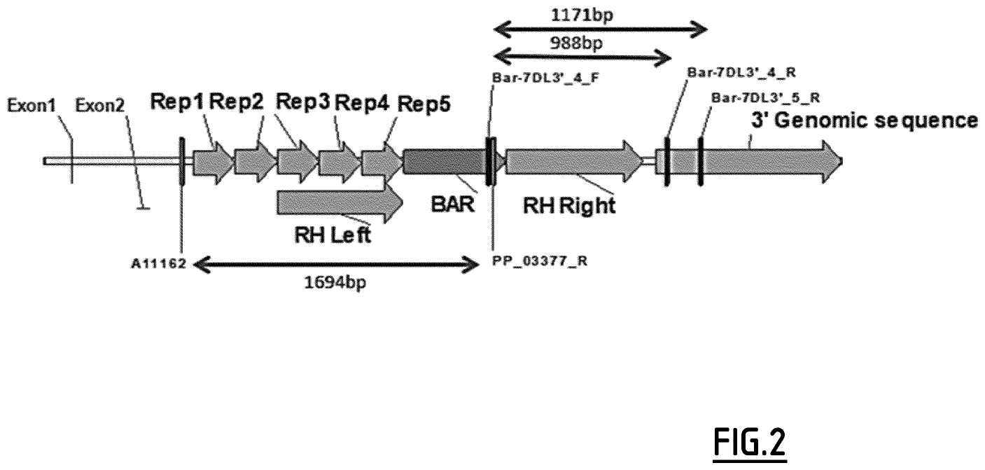

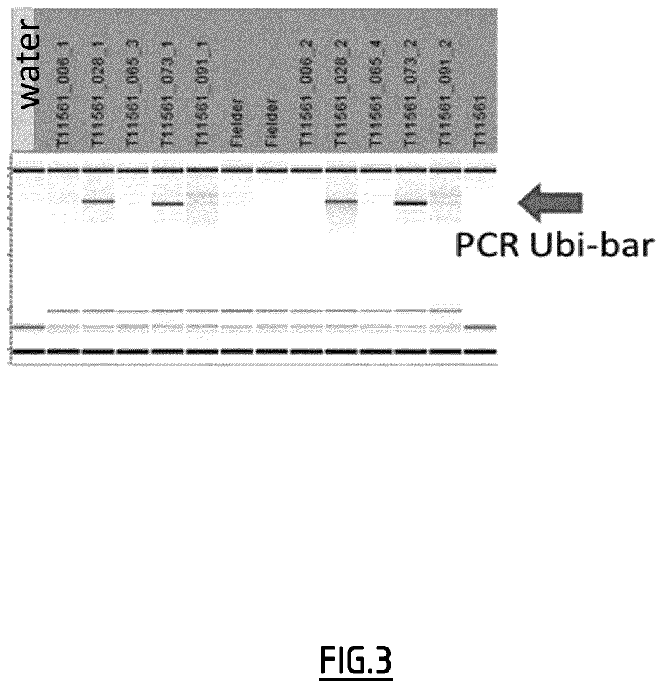

92 independent transformation events on Kanamycin selection (365 plants with sister plants) were obtained and T1 seed harvested. T1 progeny were sown and sprayed 2x with BASTA to look for resistant plants (data not shown). Several T1 families exhibited BASTA resistance (Table 1). 2 T1 plants of 55 T1T11561_028 events exhibited full resistance (data not shown). Molecular analysis of these plants by PCR (FIGS. 2 and 3) and DNA sequencing showed that these plants had the predicted insertion of Bar into the Ubi7D gene. Both left and right junction sequences were as predicted indicating insertion of Bar by a double homologous recombination event.

TABLE 1
Scoring of T0 progeny, from T0 selected
plants, after 2 sprays of BASTA.

| | | | | Resis- | % | Level of |
|---|---|---|---|---|---|---|
| | T0 | Sister | T1 seed | tant | Resis- | BASTA |
| Line | Selection | plant | sown | plants | tance | R on T1 |
| T11561_002 | Kan | N | 76 | 1 | 1% | med |
| T11561_006 | Kan | N | 86 | 4 | 5% | low |
| T11561_013 | Kan | N | 33 | 1 | 3% | med |
| T11561_014 | Kan | N | 28 | 14 | 50% | med |
| T11561_017 | Kan | N | 59 | 1 | 2% | low |
| T11561_028 | Kan | N | 55 | 2 | 4% | good |
| T11561_030 | Kan | N | 43 | 1 | 2% | low |
| T11561_056 | Kan | N | 88 | 1 | 1% | med |
| T11561_059 | Kan | N | 50 | 1 | 2% | low |
| T11561_063 | Kan | N | 23 | 1 | 4% | low |
| T11561_065 | Kan | N | 27 | 7 | 26% | low |
| T11561_067 | Kan | N | 72 | 36 | 50% | good |
| T11561_069 | Kan | N | 59 | 2 | 3% | low |
| T11561_073 | Kan | Y | 30 | 11 | 37% | good |
| T11561_074 | Kan | Y | 28 | 11 | 39% | good |
| T11561_075 | Kar | Y | 28 | 9 | 32% | good |
| T11561_078 | Kan | N | 20 | 2 | 10% | low |
| T11561_091 | Kan | N | 105 | 57 | 54% | good |
| T11561_093 | Basta Evnt1 | Y | 70 | 6 | 9% | low |
| T11561_094 | Basta Evnt1 | Y | 64 | 39 | 61% | low |
| T11561_095 | Basta Evnt2 | Y | 59 | 38 | 64% | good |
| T11561_096 | Basta Evnt2 | Y | 67 | 35 | 52% | good |
| T11561_097 | Basta Evnt2 | Y | 44 | 26 | 59% | good |
| T11561_098 | Basta Evnt2 | Y | 40 | 19 | 48% | good |

Low: Plant with significant BASTA herbicide damages;
good: plant with no damage;
med: plant with intermediate damages

### Example 2: Use of the TaUbi Landing Pad to Generate Cytoplasmic Male Sterility (CMS) Restorer Plants

It is a goal of wheat seed companies to move towards the sale of hybrid wheat, since hybrid varieties usually outperform inbreds. Since wheat is dioecious and largely autogamous the production of hybrid seed requires systems to facilitate crossing and reduce the cost of hybrid seed production. Such a system is the use of male-sterile ‘female’ plant line crossed to a male fertile line such that all the seed harvested from the female, male-sterile plants will be F1 hybrid seed. Male-sterile plants can be produced using cytoplasmic male sterility (CMS) where the female plant carries ‘defective’ mitochondria that often express novel ORFs leading to the production of no or defective pollen. Use of CMS systems for hybrid seed production requires that the male line used in the hybrid seed production cross carries a nuclear gene or genes that repair the defective mitochondria in the F1. This leads to full male-fertility of the F1 plants that are grown by the farmer. These nuclear genes in the male line are referred to as CMS restorer genes. One potential CMS system for hybrid wheat production is that using T. timopheevii CMS (WO 2019/086510 A1 or PCT/EP2022/064472). A drawback of this system is that a combination of several restorer genes (Rf1, Rf3, Rf4 and Rf7) is required to give full male fertility to the F1. For the breeder this makes the system more complex to use since each male line has to be converted to contain 3 or 4 independently segregating restorer genes. It is thus desirable to identify or create a single effective restorer locus.

The T. timopheevii CMS restorer gene Rf3 has been identified as a PPR protein on Chr1B referred to as RFL29 (TraesCS1B01G038500) (WO 2019/086510 A1). This gene is present in most wheat lines such as Chinese Spring though its level of expression is very low as measured by RNAseq data. There are at least 3 RFL29 variants in wheat. RFL29b (SEQ ID NO: 15 encoding SEQ ID NO: 16), present in Chinese Spring is a less effective restorer than the RFL29a allele (SEQ ID NO: 17 encoding SEQ ID NO: 18) found in lines such as Spelt. Some lines such as Fielder contain an inactive RFL29 variant, RFL29c, with a frameshift in the coding region. To determine if RFL29-mediated fertility restoration can be improved, RFL29a and RFL29b were placed under the control of the strong ZmUbiquitin (ZmUbi) promoter and transformed into a wheat line containing T. timopheevii CMS. Full male fertility was observed in single copy T-DNA transformants.

Similarly, Rf1 has been found to be a PPR gene (RFL79) (WO 2019/086510 A1) on Chr1A (SEQ ID NO: 19 encoding SEQ ID NO: 20). As for RFL29, overexpression of RLF79 under the strong ZmUbi promoter restores full male fertility in a wheat line containing T. timopheevii CMS.

Wheat 7DL Polyubiquitin::RFL29 and polyubiquitin::RFL79 fusions also restore male fertility to a wheat line containing T. timopheevii CMS when expressed as a transgene from the maize Ubi promoter or the wheat Ubi promoter. This is the case when the RFL genes are expressed as 5′ or 3′ fusions to polyubiquitin (table 2). In the case of 5′ fusions, the RFL29a or RFL79 sequence has an added 3′ ubiquitin tail of 14 amino acids (Walker and Vierstra (2007) which are the C-terminal amino acids of the first Ubi repeat in Ubi7DL (SEQ ID NO: 21, SEQ ID NO: 22, SEQ ID NO: 23 and SEQ ID NO: 24).

TABLE 2
Restoration of fertility in a Wheat T. timopheevii
CMS line transformed with TaUbi7DL::RFL fusion genes

| | | | | Restoration | Fertile |
|---|---|---|---|---|---|
| Strain | Promoter | GOI | Terminator | efficiency (%) | plants |
| T11635 | ZmUbi | RH_TaUbi7D_TaRFL29a | SbHSP | 77 | 14/18 |
| | | (SEQ ID NO: 21) | | | |
| T11634 | ZmUbi | TaRFL29a_RH_TaUbi7D | SbHSP | 82 | 18/22 |
| | | (SEQ ID NO: 22) | | | |
| T11549 | ZmUbi | RH_TaUbi7D_TaRFL79 | SbHSP | 93 | 42/45 |
| | | (SEQ ID NO: 23) | | | |
| T11548 | ZmUbi | TaRFL79_RH_TaUbi7D | SbHSP | 61 | 19/31 |
| | | (SEQ ID NO: 24) | | | |

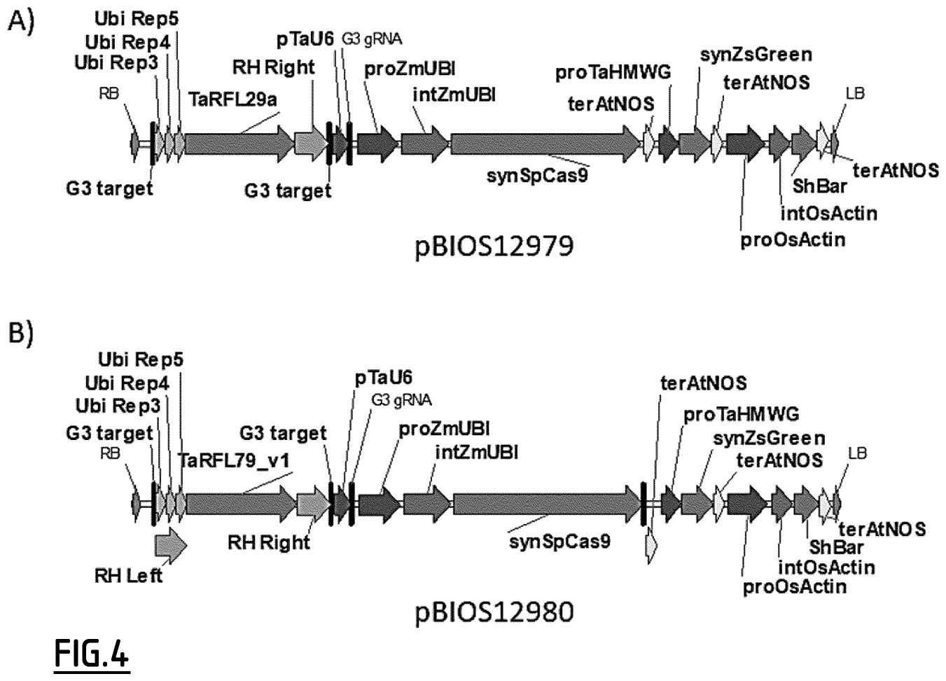

These results demonstrate the ability of a maize Ubi promoter to drive sufficient expression of the fertility restorer sequence to have fertile plants, but also to have a fusion protein properly processed to restore sterility. The restoration of fertility implies that the processed protein is correctly imported into the mitochondria where it will restore mitochrondrial function and fertility. Thus, integration of RFL29 or RFL79 or both into the wheat polyubiquitin landing pad should create a single locus T. timopheevii CMS restorer gene. Transformation constructs to achieve this are shown in FIG. 4 and are transformed into a wheat line containing T. timopheevii CMS. The donor fragment for homologous recombination (HR) of RFL29a at the wheat Ubi7DL Landing Pad comprises of the Ubi7DL left HR region (repeats 3, 4 and 5): RFL29a: Ubi7DL terminator (right HR region) (SEQ ID NO: 26). This HR region is flanked by G3 gRNA sites. A similar donor fragment for integration of RFL79 at the wheat Ubi7DL Landing Pad was constructed (SEQ ID NO: 27).

Progeny of the TO transformed lines are screened by PCR to identify GT events where the RFL29a or RFL79 gene has been integrated into the TaUbi7DL Landing Pad. These plants are fertile. The full restoration of fertility by plants expression a single copy of these both gene in the landing pad context confirms the potential of the method to give a high level of expression of a sequence introduced in this context.

### Example 3: Co-Expression of CMS Restorer Genes from a Wheat Polyubiquitin Landing Pad in Order to Restore Male Fertility

More than one gene can be integrated into a polyubiquitin Landing Pad. To improve the T. timopheevii CMS system, it would be advantageous to express the two restorer genes RFL29a and RFL79 (see example 2) from the Polyubiquitin Landing Pad. This would assure sufficient expression of each restorer and in addition create a single locus that can be introgressed by the breeder to convert wheat lines into T. timopheevii CMS restorer lines. In example 2, it was shown that an N-terminal fusion of RFL29a to polyubiquitin when expressed in CMS wheat can restore male fertility (Table 2, strain T11634). Thus, a donor fragment for homologous recombination (HR) at the wheat Ubi7DL Landing Pad comprises the Ubi7DL right RH region (repeats 3, 4 and 5): RFL29a: 14aa of the C-terminus of Ubi7DL repeat1: Ubi7DL repeat1: RFL79: Ubi7DL terminator (right HR region) (SEQ ID NO: 28). This HR region is flanked by G3 gRNA sites.

As in example 2, the HR donor region plus flanking G3 gRNA sites is assembled into a plant binary vector for Agrobacterium-mediated transformation of a wheat line containing T. timopheevii CMS. This binary vector contains a pZmUbi:Cas9 expression cassette, a pTaU6 G3 gRNA expression cassette (SEQ ID NO: 25) and a bar selectable marker cassette.

Progeny of the TO transformed lines are screened by PCR to identify GT events where the RFL29a and RFL79 genes have been integrated into the TaUbi7DL Landing Pad. These plants are fertile.

### Example 4: Use of TaUbi Landing Pad to Generate Herbicide Tolerant Wheat Plants

The control of weeds is a major agronomical goal for the production of wheat with respect to competition for water and nutrients and to avoid pollution of seed stocks with undesired weed seeds. The use of chemical herbicide is preferred to mechanical approaches in the sense that it avoids damages to soil structure and erosion. There exists today a large panoply of chemical herbicides which have been developed to this purpose. Ideally wheat should be tolerant to numerous types of herbicides and not just to a single type to avoid the presence of weed types tolerant to a given class of herbicides.

Working with and introgressing several types of herbicide tolerance genes is a real challenge to breeders, and it would be desirable to have those genes sufficiently expressed and stacked into a single locus. To this aim, the Ubiquitin locus is well adapted since it produces a polyprotein which is subsequently cleaved into single units by cytoplasmic proteases. Furthermore, this locus is expressed in a constitutive fashion and at levels suited to provide good herbicide tolerance.

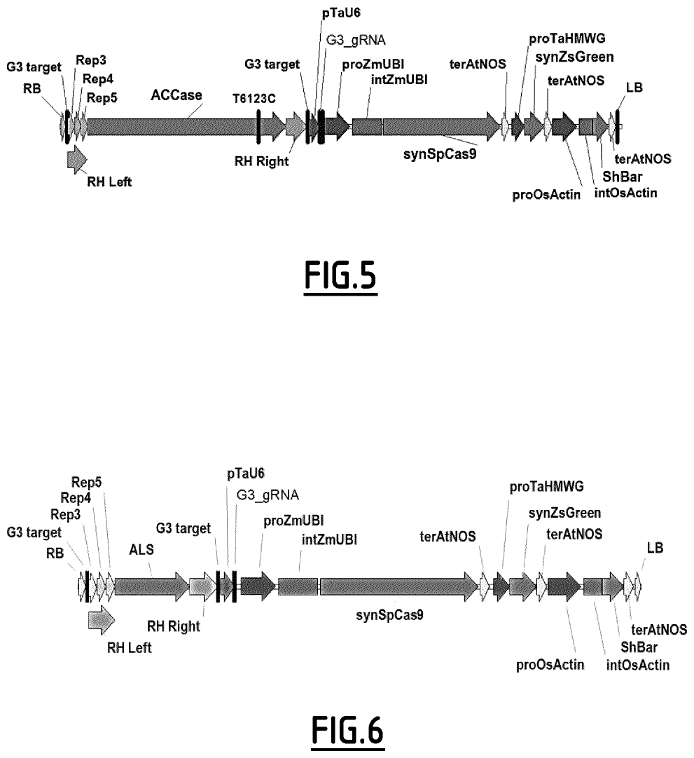

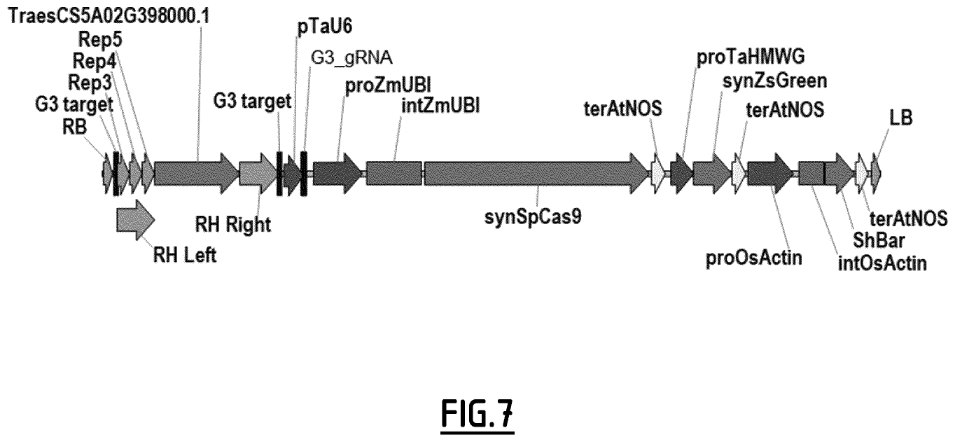

Examples of herbicide tolerance genes which may be expressed in this way may be (but not limited to) the wheat acetyl-CoA Carboxylase (ACCase) (ACCase chrA SEQ ID NO: 29 encoding SEQ ID NO: 30, ACCase chrB SEQ ID NO: 31 encoding SEQ ID NO: 32, ACCase chrD SEQ ID NO: 33 encoding SEQ ID NO: 34) comprising the CoAXium mutation Ala2004Val (U.S. Pat. No. 9,578,880_B2) (or other mutations from EP2473022_B1) (FIG. 5), wheat Acetolactate Synthase (ALS) (FIG. 6) mutated individually in amino-acids (numbering according to the reference Arabidopsis ALS protein encoded by AT3G48560) A122, P197, A205, D376, W574, S653 or any of the 4 mutations P197, A205, D376 or W574 in combination between them or with A122 or S653 (ALS chr6A SEQ ID NO: 35 encoding SEQ ID NO: 36, ALS chr6B SEQ ID NO: 37 encoding SEQ ID NO: 38, ALS chr6D SEQ ID NO: 39 encoding SEQ ID NO: 40), or a gene encoding a cytochrome P450 involved in herbicide detoxification. An example for a cytochrome P450 enzyme may be the wheat orthologous gene (TraesCS5A02G398000, SEQ ID NO: 41 encoding SEQ ID NO: 42) which is orthologous to a P450 gene from Lolium rigidum described by Han et al. (2021) (FIG. 7), another example is the one described in EP22306134.2.

These herbicide resistance genes are cloned into a plant binary vector essentially identical to pBIOS12979 (example 2) apart from the replacement of RFL29a with the herbicide resistance gene. The T-DNA regions of these plant binary vectors is shown in FIGS. 5, 6 and 7. These plant binary vectors are transferred into Agrobacterium and used in Agrobacterium-mediated transformation of the wheat variety Fielder. Progeny of the TO transformed lines are screened by PCR to identify GT events where the herbicide-resistance genes have been integrated into the TaUbi7DL Landing Pad. These plants are screened for herbicide resistance using an appropriate herbicide.

### Example 4bis: Over-Expression of a Mutated ALS Gene Fused to the Polyubiquitin Coding Region

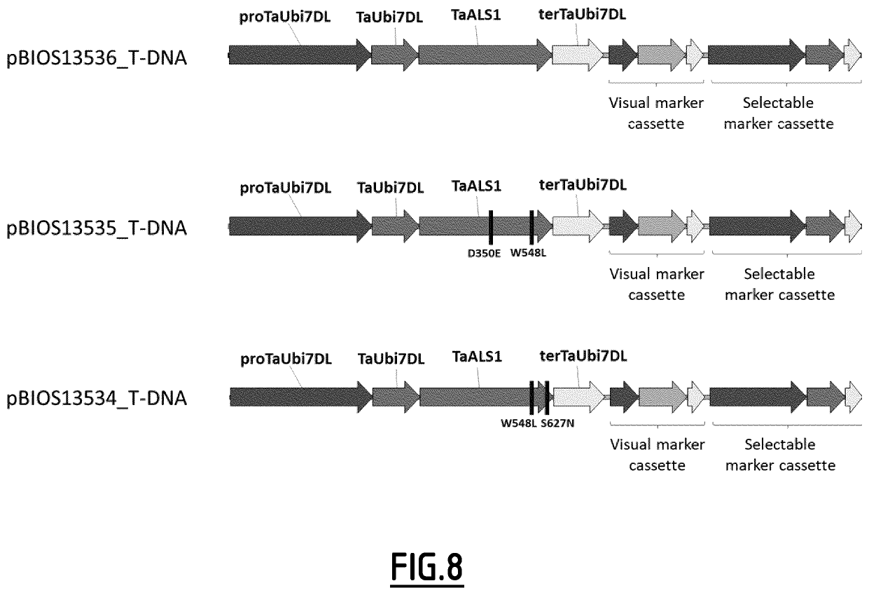

The coding region of the wheat ALS1 gene (TraesFLD6D01G329900) was fused to the polyubiquitin gene on chromosome 6D from wheat genotype Fielder (TraesFLD7D01G490700) between the 3′end of the coding region and the terminator. The introduced ALS1 coding region was either the wild-type sequence, or a mutated sequence with amino-acids D350E and W548L (SEQ ID NO: 79) or W548L and S627N (SEQ ID NO: 80). Those amino-acids are equivalent to the Arabidopsis ALS amino-acids D376, W574 or S653. The resulting fragments (Ubi7D_promoter::Ubi7D_cds::ALS1 cds::Ubi7D_terminator, SEQ ID NO: 85) were introduced via a Golden Gate reaction into the destination binary plasmid pBIOS10746 which is a derivative of the binary vector pMRT (WO200101819A3), FIG. 8. The final plasmids pBIOS13536 (fusion with wild-type ALS1), pBIOS13535 (fusion with D350E, W548L mutated ALS1) or pBIOS13534 (fusion with W548L, S627N mutated ALS1) were transformed into Agrobacterium EHA105.

Fielder wheat cultivar was transformed with these Agrobacterium strains essentially as described by WO2000/063398. Wheat transgenic events were generated for each construct described above. All wheat transgenic plants were grown in a glasshouse under standard wheat growth conditions (16 h of light period at 20° C. and 8 h of dark period at 15° C. with constant 60% humidity).

### ALS1 Inhibiting Herbicide Assay

To assay for ALS1-inhibiting herbicide of the sulfonylurea family nicosulfuron, T1 plants (progeny of transformed wheat plants) were grown in the glasshouse until the growth stage BBCH13 (3 developed leaves) and sprayed with a solution of nicosulfuron (Pampa herbicide) at a concentration of 0.1 g/L and a spraying rate equivalent to what is used by farmers (600 L/ha).

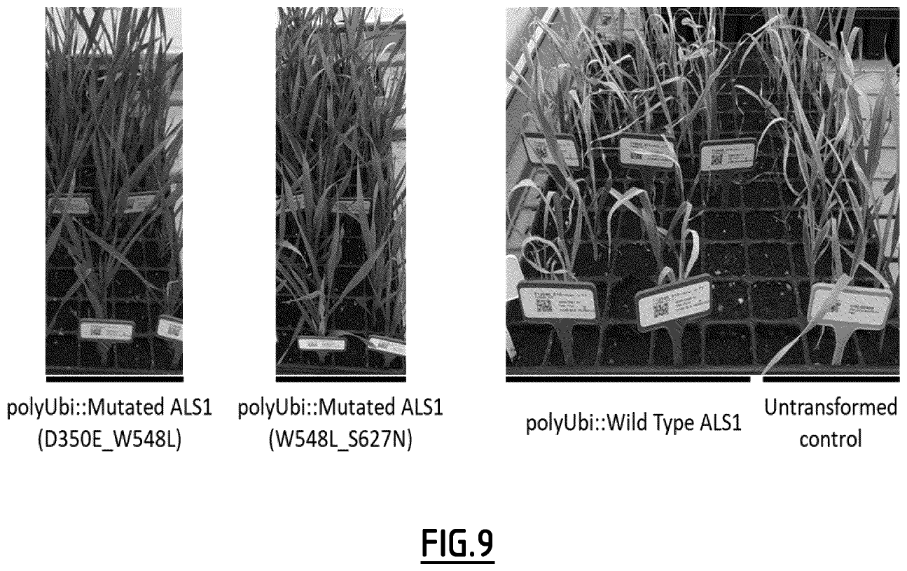

Herbicide effect was evaluated between 8 and 16 days after herbicide treatment (FIG. 9). Plants transformed with the mutated ALS1 gene fused to the polyubiquitin gene were resistant to nicosulfuron treatment, whereas untransformed plants or plants transformed with the wild-type ALS1 gene fused to the polyubiquitin gene were susceptible to the herbicide.

These results demonstrate the ability of the wheat Ubi promoter in this context to drive an expression strong enough to obtain resistance to the herbicide and also that the fusion with ubi sequences allows a correct processing of the protein allowing it to be correctly targeted to the chloroplast.

ALS1 inhibiting herbicides include molecules belonging to various families like Sulfonylurea (nicosulfuron), Imidazolinone (imazamox), Triazolinones (carfentrazone-ethyl), or Triazolopyrimidine (florasulam). Weeds tolerant to those herbicides were identified in nature and tolerance shown to result from mutations in their ALS1 gene at amino-acids D376 or W574 (Arabidopsis protein position). The introduced ALS1 mutations in wheat correspond to those changes and the transformed plants over-expressing those mutations are tolerant to those different herbicides.

### Example 5: Use of a ZmUbi Landing Pad to Generate Insect-Resistant Plants

Maize line B73 has two polyubiquitin genes that are highly and ‘constitutively’ expressed, Zm00001d053838 on Chr4 (SEQ ID NO: 43) and Zm0001d015327 (SEQ ID NO: 44) on Chr5 (genome B73 v4). Equivalent genes in A188 on Chr4 (SEQ ID NO: 45) and Chr5 (SEQ ID NO: 46) can be identified by sequence homology to B73. The promoter of the ZmUbiChr5 gene is widely used in plant transgenesis as a strong and constitutive promoter. Specific Cas9 gRNA can be identified that create a double strand break (DSB) near to the Stop codon of ZmUbiChr4 or ZmUbiChr5. Both ZmUbiChr4 and ZmUbiChr5 can be used as Landing Pads. ZmUbiChr4 is located near to the telomere of Chr4 thus gene insertions into this Landing Pad may be easier to introgress into other maize varieties than for insertions in ZmUbiCh5 which is near to the centromere of Chr5. However, ZmUbiChr5 seems to be more highly expressed thus depending on the application, one or other of the Landing Pads may be more appropriate.

A guide targeting an analogous position to that of the wheat gRNA3 of example 1 and example 2 in ZmUbiChr4, gRNA31 (SEQ ID NO: 47) can be used to create a DSB adjacent to the Stop codon of ZmUbiChr4 in both B73 and A188. Similarly, gRNA20 ((SEQ ID NO: 48) can be used to create a DSB adjacent to the Stop codon of ZmUbiChr5 in both B73 and A188. As in examples 1 and 2 regions flanking the stop codon of ZmUbiChr4 can be used are homology regions for homologous recombination of a coding region of interest into the ZmUbiChr4 Landing Pad. Also, regions flanking the stop codon of ZmUbiChr5 can be used are homology regions for homologous recombination of a coding region of interest into the ZmUbiChr5 Landing Pad.

An example of a coding region of interest is a gene for insect resistance Bt Cry1Ac (SEQ ID NO: 50 encoding SEQ ID NO: 51) that is introduced into the ZmUbiChr5 Landing Pad. ZmUbiChr5 homology flanking regions are cloned upstream and downstream of a maize-codon-optimised Cry1Ac gene which is in turn flanked by target sequences for gRNA20 (The gRNA20 sites contain 6 bp upstream and 6 bp downstream of ZmUbiChr5 sequences flanking the gRNA20 target to help maintain the context of the gRNA20 target). The Cry1Ac gene can also contain sub-cellular targeting signals. SEQ ID NO: 52 shows a Cry1Ac gene that has an N-terminal chloroplast targeting signal from Rubisco Activase (RCA). The homologous recombination Cry1Ac and RCA-Cry1Ac repair fragments (SEQ ID NO: 53 and SEQ ID NO: 54) are then cloned into a plant binary vector containing a rice Actin promoter-BAR nos terminator selectable marker gene together with a ZmUbi promoter-Cas9-Nos terminator cassette and a Maize U6-gRNA20 cassette. The resulting binary plasmids are transferred to Agrobacteria and used in A188 maize transformation using a standard maize Agrobacterium protocol (Ishida et al., 1996)

Progeny of the TO transformed lines are screened by PCR to identify GT events where the Cry1 Ac or RCA-Cry1 Ac genes has been integrated into the ZmUbi Chr5 Landing Pad.

### Example 6: Use of a BnUbi Landing Pad to Generate Fertility Restorer Plants

The Ogura CMS system is used by seed companies to produce hybrid F1 rapeseed. This system requires a fertility restorer gene, Rfo, that originates from an introgression from radish (Raphanus sativus) (Qui et al., 2014). The original introgression also contained agronomically undesirable linked traits such as increased pod shatter and glucosinolate levels. Thus, considerable effort has been undertaken to reduce the size of the introgression which had proven difficult probably due to limited homology with B. napus or to create new introgressions (see Wang et al., 2020). Since the restorer gene Rfo (SEQ ID NO: 55 encoding SEQ ID NO: 56) has been identified and functionally characterized (see Qui et al., 2014) an alternative is to introduce Rfo into a polyubiquitin Landing Pad. As such there will be good expression of Rfo without any effects due to linkage drag.

The B. napus gene expression site (Brassica EDB) described in Chao et al, (2020) was examined to identify polyubiquitin genes with a good constitutive expression. Of the 13 polyubiquitin genes in Brassica EDB three appeared to have high and relatively constitutive expression (BnaA09g19810D (SEQ ID NO: 57), BnaC09g21810D (SEQ ID NO: 58) and BnaA08g30590D (SEQ ID NO: 59). BnaA09g19810D was chosen as a Landing Pad, the other two also being suitable candidates (in addition, depending on the desired expression pattern of the Gene of Interest the other polyubiquitin genes can be used as Landing Pads). The B. napus var. Westar BnaA09g19810D genomic sequence (SEQ ID NO: 60) (BnUbiA09) was identified by homology with the BnaA09g19810D sequence. A guide targeting an analogous position to that of the wheat gRNA3 of example 1 and example 2 in BnUbiA09; gRNA16 (SEQ ID NO: 61) can be used to create a DSB adjacent to the Stop codon of BnUbiA09. As in examples 1 and 2 regions flanking the stop codon of BnUbiA09 can be used as homology regions for homologous recombination of a coding region of interest into the BnUbiA09 Landing Pad. Homology flanking regions are cloned upstream and downstream of the Rfo genomic coding region which is in turn flanked by target sequences for gRNA16 (The gRNA16 sites contain 6 bp upstream and 6 bp downstream of BnUbiA09 sequences flanking the gRNA16 target to help maintain the context of the gRNA16 target). The homologous recombination Rfo cassette (SEQ ID NO: 63) is then cloned into a plant binary vector containing Nos nptII nos terminator selectable marker gene together with a 35S promoter (SEQ ID NO: 64)-Cas9 (SEQ ID NO: 65)-CaMV terminator cassette and an Arabidopsis U6 (SEQ ID NO: 62)-gRNA16 cassette. The resulting binary plasmid is transferred to Agrobacteria and used in B. napus var. Westar transformation using a standard B. napus agrobacterium protocol (Moloney et al, 1989). Progeny of the TO transformed lines are screened by PCR to identify GT events where the Rfo gene has been integrated into the BnUbiA09 Landing Pad.

### REFERENCES

Avila-Garcia W V, Sanchez-Olguin E, Hulting A G and Mallory-Smith C (2012) Target-site mutation associated with glufosinate resistance in Italian ryegrass (Lolium perenne L. ssp. multiflorum). Pest Management Science 68 (9):1248-1254. doi.org/10.1002/ps.3286
Barret M (1995) Metabolism of herbicides by cytochrome P450 in corn. In Metabolism of Herbicides Vol 12, No 3-4, 299-315.
Brazier-Hicks M, Franco-Ortega S, Watson P, Rougemont B, Cohn J, Dale R, Hawkes T R, Goldberg-Cavalleri A, Onkokesung N and Edwards R (2022) Characterization of cytochrome P450s with key roles in determining herbicide selectivity in maize. ACS Omega 7:17416-17431.
Chao H, Li T, Luo C, Huang H, Ruan Y, Li X, Niu Y, Fan Y, Sun W, Zhang K, Li J, Qu C, Lu K (2020). Brassica EDB: A Gene Expression Database for Brassica Crops. Int J Mol Sci.13; 21(16):5831. doi: 10.3390/ijms21165831.
Dimaano N G and Iwakami S (2020) Cytochrome P450-mediated herbicide metabolism in plants: current understanding and prospects. Pest Manag Sci 77 (1): 22-32. doi.org/10.1002/ps.6040
Han H, Yu Q, Beffa R, Gonzalez S, Maiwald F, Wang J and Powles S B (2021) Cytochrome P450 CYP81A10v7 in Lolium rigidum confers metabolic resistance to herbicides across at least five modes of action. Plant J. 105:79-92. doi: 10.1111/tpj. 15040
Hondred D, Walker J M, Mathews D E, Vierstra R D. (1999). Use of ubiquitin fusions to augment protein expression in transgenic plants. Plant Physiol. 119:713-24. doi: 10.1104/pp. 119.2.713.
Fauser F, Roth N, Pacher M, I Ig G, Sánchez-Fernández R, Biesgen C, Puchta H. (2012) In planta gene targeting. Proc Natl Acad Sci USA. 109:7535-40. doi: 10.1073/pnas. 1202191109.
Ishida et al., (1996) Nat. Biotechnol., 14:745-750
Moloney et al., (1989) Plant Cell Reports, 8: 238-242.
Qin X, Warguchuk R, Arnal N, Gaborieau L, Mireau H, Brown G G. (2014). In vivo functional analysis of a nuclear restorer PPR protein. BMC Plant Biol. 14:313. doi: 10.1186/s12870-014-0313-4.
Tang Z, Zhang L, Xu C, Yuan S, Zhang F, Zheng Y, Zhao C (2012). Uncovering small RNA-mediated responses to cold stress in a wheat thermosensitive genic male-sterile line by deep sequencing. Plant Physiol. 159:721-738. DOI: https://doi.org/10.1104/pp. 112.196048.
Walker, J M and Vierstra, R D (2007) A ubiquitin-based vector for the co-ordinated synthesis of multiple proteins in plants. Plant Biotechnol J. 5:413-21. doi: 10.1111/j.1467-7652.2007.00250.x
Wang T, Guo Y, Wu Z, Xia S, Hua S, Tu J, Li M, Chen W. (2020). Genetic characterization of a new radish introgression line carrying the restorer gene for Ogura CMS in Brassica napus. PLOS One. 15 (7): e0236273. doi: 10.1371/journal.pone.0236273. eCollection 2020.
Zhang C, Yu Q, Han H, Yu C, Nyporko A, Tian X, Beckie H and Powles S (2022) A naturally evolved mutation (Ser59Gly) in glutamine synthetase confers glufosinate resistance in plants. J Exp Bot 73 (7): 2251-2262. doi.org/10.1093/jxb/erac008
U.S. Pat. No. 9,578,880_B2. ACETYL CO-ENZYME A CARBOXYLASE HERBICIDE RESISTANT PLANTS
EP2473022_B1. HERBICIDE-TOLERANT PLANTS
WO 2019/086510 A1. WHEAT COMPRISING MALE FERTILITY RESTORER ALLELES WO 2021/000870 A1 GLUTAMINE SYNTHETASE MUTANT HAVING GLUFOSINATE AMMONIUM RESISTANCE AND APPLICATION THEREOF AND CULTIVATION METHOD THEREFOR

1. A vector suitable for a targeted integration of at least one gene of interest in 5′ or 3′ of a polyubiquitin gene in a plant, wherein said vector comprises a repair DNA comprising from 5′ to 3′:
a first gRNA target,
a left ubiquitin-like region,
at least one gene of interest,
a right ubiquitin-like region, and
a second gRNA target.

2. The vector according to claim 1, further comprising:
at least one CRISPR-Cas endonuclease expression cassette and/or
at least one gRNA expression cassette encoding a gRNA able to recognize a region in 3′ or 5′ of the polyubiquitin gene.

3. The vector according to claim 2, wherein the vector comprises a single gRNA expression cassette.

4. The vector according to claim 1, wherein the gene of interest is selected from the group consisting of a herbicide tolerance gene, an insect resistance gene, a fungal resistance gene, a bacterial resistance gene, a stress resistance gene, a gene involved in reproductive capability, a gene involved in performance in the fields, a gene involved in performance in an industrial process and a gene involved in nutritional value of a plant.

5. The vector according to claim 1, wherein the gene of interest is selected from the group consisting of BAR gene, ALS gene, GS, cyt P450 gene, RFL29a gene, RFL79 gene, Rfo gene, Cry1Ac gene and RCA-Cry1Ac gene.

6. A plant cell or plant tissue comprising at least one gene of interest inserted in 5′ or in 3′ of the polyubiquitin gene, obtained by transformation with a vector, wherein said vector comprises a repair DNA comprising from 5′ to 3′:
a first qRNA target,
a left ubiquitin-like region,
at least one gene of interest,
a right ubiquitin-like region, and
a second qRNA target.

7. The plant cell or plant tissue according to claim 6, which is a protoplast, apical meristem, cotyledon, embryo, pollen or microspores.

8. A plant comprising at least one gene of interest inserted in 5′ or in 3′ of the polyubiquitin gene, obtained by transformation with a vector, wherein said vector comprises a repair DNA comprising from 5′ to 3′:
a first qRNA target,
a left ubiquitin-like region,
at least one gene of interest,
a right ubiquitin-like region, and
a second qRNA target.

9. The plant cell or plant tissue according to claim 6, wherein said plant cell or plant tissue comprises at least one polyubiquitin gene.

10. A progeny plant of a plant according to claim 8, wherein said progeny plant comprises at least one gene of interest inserted in 5′ or in 3′ of the polyubiquitin gene.

11. A method for targeted insertion of at least one gene of interest in 5′ or 3′ of a polyubiquitin gene in a plant genome, comprising:
a. transformation of a plant cell or plant tissue with at least one vector, wherein said vector comprises a repair DNA comprising from 5′ to 3′:
a first gRNA target,
a left ubiquitin-like region,
at least one gene of interest,
a right ubiquitin-like region, and
a second qRNA target,
to obtain a transformed plant cell or plant tissue, and
b. the regeneration of the plant from the transformed plant cell or plant tissue.

12. The method of claim 11, wherein at least one CRISPR-Cas endonuclease expression cassette is provided by said vector or in a separate vector and wherein at least one gRNA expression cassette is provided by said vector or in a separate vector.

13. A method for expressing at least one protein of interest in a plant, comprising the steps of the method according to claim 11, wherein said gene of interest codes for said protein of interest.

14. A method for expressing at least one gene of interest in a plant, in a plant cell or in a plant tissue, comprising transforming a plant cell or a plant tissue with a vector, wherein said vector comprises a repair DNA comprising from 5′ to 3′
a first qRNA target,
a left ubiquitin-like region,
at least one gene of interest,
a right ubiquitin-like region, and
a second gRNA target.

15. A method of identifying a plant comprising at least one gene of interest inserted in 5′ or 3′ of a polyubiquitin gene, wherein said method comprises:
extracting the DNA, RNA or proteins of a plant,
detecting the presence of a DNA comprising said at least one gene of interest inserted in 5′ or 3′ of a polyubiquitin gene and/or the presence of a RNA transcript from said DNA, and
optionally, detecting the presence of a protein encoded by said at least one gene of interest.

16. The plant according to claim 8, wherein said plant comprises at least one polyubiquitin gene.
